SEQUENCE LISTING (26 sequences)

SEQ ID NO: 1 - protein, 153 aa, Artificial Sequence
  example interleukin-2 (IL-2) sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - DNA, 462 bp, Artificial Sequence
  example nucleotide sequence encoding IL-2
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 3 - protein, 177 aa, Artificial Sequence
  example interleukin-7 (IL-7) sequence
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - DNA, 534 bp, Artificial Sequence
  example nucleotide sequence encoding IL-7
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 5 - protein, 162 aa, Artificial Sequence
  example interleukin-15 (IL-15) sequence
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - DNA, 489 bp, Artificial Sequence
  example nucleotide sequence encoding IL-15
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 7 - DNA, 588 bp, Artificial Sequence
  example woodchuck hepatitis post-transcriptional regulatory element (WPRE) sequence
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 8 - DNA, 553 bp, Artificial Sequence
  example Enhanced Transthyretin (ET) promoter sequence
cgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 9 - DNA, 335 bp, Artificial Sequence
  example albumin promoter sequence
ggcatgcttccatgccaaggcccacactgaaatgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaatgatctacagttattggttaaagaagtatattagagcgagtctttctgcacacagatcacctttcctatcaacccc

SEQ ID NO: 10 - DNA, 23 bp, Artificial Sequence
  example miR-142 target sequence
tccataaagtaggaaacactaca

SEQ ID NO: 11 - DNA, 22 bp, Artificial Sequence
  example miR-155 target sequence
cccctatcacgattagcattaa

SEQ ID NO: 12 - DNA, 21 bp, Artificial Sequence
  example miR-223 target sequence
ggggtatttgacaaactgaca

SEQ ID NO: 13 - protein, 19 aa, Artificial Sequence
  targeting ligand peptide sequence
CKNEKKNKIERNNKLKQPP

SEQ ID NO: 14 - protein, 8 aa, Artificial Sequence
  HBV core protein epitope sequence
MGLKFRQL

SEQ ID NO: 15 - protein, 12 aa, Artificial Sequence
  HBsAg epitope sequence
IPQSLDSWWTSL

SEQ ID NO: 16 - DNA, 18 bp, Artificial Sequence
  HIV sense primer
tactgacgctctcgcacc

SEQ ID NO: 17 - DNA, 17 bp, Artificial Sequence
  HIV antisense primer
tctcgacgcaggactcg

SEQ ID NO: 18 - DNA, 20 bp, Artificial Sequence
  probe sequence
atctctctccttctagcctc

SEQ ID NO: 19 - DNA, 18 bp, Artificial Sequence
  Telo sense primer
ggcacacgtggcttttcg

SEQ ID NO: 20 - DNA, 24 bp, Artificial Sequence
  Telo antisense primer
ggtgaacctcgtaagtttatgcaa

SEQ ID NO: 21 - DNA, 24 bp, Artificial Sequence
  Telo probe sequence
tcaggacgtcgagtggacacggtg

SEQ ID NO: 22 - DNA, 23 bp, Artificial Sequence
  RT-LV deltaU3 sense primer
tcactcccaacgaagacaagatc

SEQ ID NO: 23 - DNA, 19 bp, Artificial Sequence
  gag antisense primer
gagtcctgcgtcgagagag

SEQ ID NO: 24 - DNA, 20 bp, Artificial Sequence
  HBV core forward primer
taccgcctcagctctgtatc

SEQ ID NO: 25 - DNA, 21 bp, Artificial Sequence
  HBV core reverse primer
cttccaaattaacacccaccc

SEQ ID NO: 26 - DNA, 27 bp, Artificial Sequence
  HBV core probe sequence
tcacctcaccatactgcactcaggcaa

(19) United States
(12) Patent Application Publication
(10) Pub. No.: US 2022/0251161 A1
(43) Pub. Date: Aug. 11, 2022

(54) Agents and Methods for Treating Viral Infections

(71) Applicants: Ospedale San Raffaele S.R.L., Milan (IT); Fondazione Telethon, Rome (IT)

(72) Inventors: Luigi Naldini, Milan (IT); Alessio Cantore, Milan (IT); Matteo Iannacone, Milan (IT); Luca Guidotti, Milan (IT); Renato Ostuni, Milan (IT)

(21) Appl. No.: 17/614,268

(22) PCT Filed: May 28, 2020
(86) PCT No.: PCT/EP2020/064933

§ 371 Date: Nov. 24, 2021

(30) Foreign Application Priority Data
May 28, 2019 (EP) 1907493.9

(51) Int. Cl. C07K 14/55; C12N 15/86; C12N 15/113; A61K 35/17; A61K 38/17; A61P 31/20

(52) U.S. Cl. CPC C07K 14/55; C12N 15/86; C12N 15/113; A61K 35/17; A61K 38/00; A61P 31/20; C12N 2740/15043; C12N 2830/008; A61K 38/1774

(57) ABSTRACT

An interleukin which binds to IL-2 receptor (IL-2R), or a nucleotide sequence encoding therefor, wherein the interleukin or nucleotide sequence is adapted to be targeted to the liver.

### FIELD OF THE INVENTION

The present invention relates to agents for use in the treatment and prevention of viral infections. In particular, the invention relates to interleukins and vectors comprising a nucleotide sequence encoding an interleukin, and methods for treating viral liver infections using the interleukins and/or vectors.

### BACKGROUND TO THE INVENTION

Hepatitis infections, such as hepatitis B virus (HBV) infection, remain a major public health issue worldwide. For example, it has been estimated that about 248 million individuals were positive for hepatitis B surface antigen, a marker of chronic HBV infection, globally in 2010.

Chronic HBV infection is typically acquired at birth or in early childhood, and is particularly prevalent in Asian and African countries where HBV is endemic. The risk of developing chronic infection after exposure drops from ca. 90% in neonates to 1-5% in healthy adults. However, 25% of people who acquire HBV as children will develop primary liver cancer or cirrhosis as adults.

HBV is a non-cytopathic virus that replicates exclusively in hepatocytes without inducing innate immune activation. The outcome of HBV infection is mainly determined by the kinetics, breadth, vigour and effector functions of HBV-specific CD8+ T cell responses. CD8+ T cell responses to pathogens that exclusively replicate in hepatocytes, such as HBV, are known to vary from severe dysfunction to full differentiation into effector cells endowed with antiviral potential.

CD8+ T cells have a critical role in eliminating intracellular pathogens and tumours. In order to exert their defensive function, naïve CD8+ T cells need to recognise antigen (Ag), become activated, proliferate and differentiate into effector cells. This process—known as “priming”—occurs preferentially in secondary lymphoid organs, where the specialised microenvironment favours the encounter between naïve CD8+ T cells and professional Ag-presenting cells. Indeed, naïve CD8+ T cells constantly recirculate between blood and secondary lymphoid organs, while they are prevented from interacting with epithelial cells of non-lymphoid organs by the endothelial barrier.

The liver is an exception to this: the unique anatomy, slow blood flow, presence of endothelial fenestrations and absence of a basement membrane allow CD8+ T cells to sense MHC-Ag complexes and other surface ligands on hepatocytes. While priming of CD8+T cells in secondary lymphoid organs has been well characterised, the mechanisms and consequences of intrahepatic priming are less clear. In general, the liver is thought to be biased towards inducing a state of T cell unresponsiveness or dysfunction. This phenomenon underpins the acceptance of liver allografts across complete MHC mismatch barriers, the unresponsiveness toward antigens specifically expressed in hepatocytes, and the propensity of some hepatotropic viruses, such as HBV, to establish persistent infections. While the tolerogenic property of the liver has long been known, the mechanisms underlying this phenomenon, particularly in the context of HBV pathogenesis, are incompletely understood.

Current treatment for chronic HBV infection mainly relies on direct acting antiviral (DAA) drugs (e.g. tenofovir, lamivudine, adefovir, entecavir or telbivudine), which suppress virus production, but do not eradicate HBV from the liver. Accordingly, this leads to a requirement for lifelong treatment. Alternatively, some patients receive a therapy based on pegylated interferon-α (PEG-IFN-α), which whilst having limited treatment duration, has greater adverse effects.

Accordingly, there remains a significant need for improved treatments for viral infections of the liver, in particular chronic HBV infections.

### SUMMARY OF THE INVENTION

The inventors have surprisingly found that administration of interleukin-2 (IL-2) enables reinvigoration and restoration of effector responses in dysfunctional CD8+ T cells, such as against antigens specifically expressed in hepatocytes. In particular, the inventors found that IL-2 is able to increase effector responses against hepatotropic viruses, such as HBV. The inventors also found that IL-2 is able to increase effector responses in T cells from immune tolerant patients.

Moreover, the inventors' studies have revealed that local administration of IL-2 to the liver is able to increase the effector responses while avoiding the toxicity that is associated with systemic administration of IL-2. While not wishing to be bound by theory, the inventors believe that Kupffer cells may play an important role in the effects of interleukins that they have observed, and that targeting of interleukins to certain liver cell types, such as Kupffer cells, may be beneficial.

In one aspect, the invention provides an interleukin which binds to IL 2 receptor (IL-2R), or a nucleotide sequence encoding therefor, wherein the interleukin or nucleotide sequence is adapted to be targeted to the liver.

In another aspect, the invention provides interleukin-2 (IL-2), interleukin-7 (IL-7) and/or interleukin-15 (IL-15), or a nucleotide sequence encoding therefor, wherein the IL-2, IL-7 and/or IL-15, or nucleotide sequence, is adapted to be targeted to the liver.

In some embodiments, the interleukin is comprised in a nanoparticle or liposome.

In some embodiments, the nanoparticle or liposome comprises a liver-specific ligand.

In some embodiments, the nucleotide sequence encoding the interleukin is in the form of a vector adapted for liver-specific expression of the nucleotide sequence.

In some embodiments, the interleukin or nucleotide sequence is adapted to be targeted to hepatocytes. In some embodiments, the interleukin or nucleotide sequence is adapted to be targeted to liver sinusoidal endothelial cells. In some embodiments, the interleukin or nucleotide sequence is adapted to be targeted to Kupffer cells.

In one aspect, the invention provides a vector comprising a nucleotide sequence encoding an interleukin which binds to IL-2 receptor (IL-2R), wherein the vector is adapted for liver-specific expression of the nucleotide sequence.

In another aspect, the invention provides a vector comprising a nucleotide sequence encoding interleukin-2 (IL-2), interleukin-7 and/or interleukin-15 (IL-15), wherein the vector is adapted for liver-specific expression of the nucleotide sequence.

In some embodiments, the liver-specific expression is hepatocyte-specific expression. In some embodiments, the liver-specific expression is liver sinusoidal endothelial cell-specific expression. In some embodiments, the liver-specific expression is Kupffer cell-specific expression.

In some embodiments, the nucleotide sequence is operably linked to one or more expression control sequences for liver-specific expression.

In some embodiments, the nucleotide sequence is operably linked to one or more expression control sequences for hepatocyte-specific expression. In some embodiments, the nucleotide sequence is operably linked to one or more expression control sequences for liver sinusoidal endothelial cell-specific expression. In some embodiments, the nucleotide sequence is operably linked to one or more expression control sequences for Kupffer cell-specific expression.

In another aspect, the invention provides a vector comprising a nucleotide sequence encoding an interleukin which binds to IL-2 receptor (IL-2R), wherein the nucleotide sequence is operably linked to one or more expression control sequences for liver-specific expression.

In another aspect, the invention provides a vector comprising a nucleotide sequence encoding interleukin-2 (IL-2), interleukin-7 and/or interleukin-15 (IL-15), wherein the nucleotide sequence is operably linked to one or more expression control sequences for liver-specific expression.

In another aspect, the invention provides a vector comprising a nucleotide sequence encoding an interleukin which binds to IL-2 receptor (IL-2R), wherein the nucleotide sequence is operably linked to one or more hepatocyte-specific promoter and/or enhancer.

In another aspect, the invention provides a vector comprising a nucleotide sequence encoding interleukin-2 (IL-2), interleukin-7 and/or interleukin-15 (IL-15), wherein the nucleotide sequence is operably linked to one or more hepatocyte-specific promoter and/or enhancer.

In some embodiments, the nucleotide sequence is operably linked to one or more miR-142, miR-155 and/or miR-223 target sequences. In preferred embodiments, the nucleotide sequence is operably linked to one or more miR-142 target sequences.

In another aspect, the invention provides a vector comprising a nucleotide sequence encoding an interleukin which binds to IL-2 receptor (IL-2R), wherein the nucleotide sequence is operably linked to one or more miR-142 target sequences. In another aspect, the invention provides a vector comprising a nucleotide sequence encoding an interleukin which binds to IL-2 receptor (IL-2R), wherein the nucleotide sequence is operably linked to one or more miR-155 target sequences. In another aspect, the invention provides a vector comprising a nucleotide sequence encoding an interleukin which binds to IL-2 receptor (IL-2R), wherein the nucleotide sequence is operably linked to one or more miR-223 target sequences.

In some embodiments, the vector comprises 2, 3 or 4 miR-142, miR-155 and/or miR-223 target sequences operably linked to the nucleotide sequence. In preferred embodiments, the vector comprises 2, 3 or 4, preferably 4, miR-142 target sequences operably linked to the nucleotide sequence.

In some embodiments, the vector comprises a liver-specific promoter and/or enhancer operably linked to the nucleotide sequence (see, for example, Merlin, S. et al. (2019) Molecular Therapy: Methods & Clinical Development 12: 223-232).

In some embodiments, the vector comprises a hepatocyte-specific promoter and/or enhancer operably linked to the nucleotide sequence.

In some embodiments, the hepatocyte-specific promoter is selected from the group consisting of an ET promoter, albumin promoter, transthyretin promoter, alpha1-antitrypsin promoter and apoE/alpha1-antitrypsin promoter. In preferred embodiments, the promoter is an ET promoter.

In some embodiments, the vector comprises a liver sinusoidal endothelial cell-specific promoter and/or enhancer operably linked to the nucleotide sequence.

In some embodiments, the liver sinusoidal endothelial cell-specific promoter is selected from the group consisting of a vascular endothelial cadherin (VEC) promoter, intercellular adhesion molecule 2 (ICAM2) promoter, foetal liver kinase 1 (Flk1) promoter and Tie2 promoter.

In some embodiments, the vector comprises a Kupffer cell-specific promoter and/or enhancer operably linked to the nucleotide sequence.

In some embodiments, the Kupffer cell-specific promoter is a CD11 b promoter.

In some embodiments, the vector comprises one or more liver- or hepatocyte-specific cis-acting regulator modules (CRMs, see Merlin, S. et al. (2019) Molecular Therapy: Methods & Clinical Development 12: 223-232), for example CRM8.

In some embodiments, the nucleotide sequence is operably linked to one or more miR-142, miR-155 and/or miR-223 target sequences, and a liver-specific promoter and/or enhancer. In some embodiments, the nucleotide sequence is operably linked to one or more miR-142 target sequences, and a liver-specific promoter and/or enhancer.

In some embodiments, the nucleotide sequence is operably linked to one or more miR-142, miR-155 and/or miR-223 target sequences, and a hepatocyte-specific promoter and/or enhancer. In some embodiments, the nucleotide sequence is operably linked to one or more miR-142 target sequences, and a hepatocyte-specific promoter and/or enhancer. In preferred embodiments, the nucleotide sequence is operably linked to one or more miR-142 target sequences, and an ET promoter. In particularly preferred embodiments, the nucleotide sequence is operably linked to 4 miR-142 target sequences, and an ET promoter.

In some embodiments, the nucleotide sequence is operably linked to one or more miR-122 target sequence. In some embodiments, the nucleotide sequence is operably linked to one or more miR-199a target sequence. In some embodiments, the nucleotide sequence is operably linked to one or more miR-126 target sequence. Use of a miR-122 target sequence may repress expression of the nucleotide sequence in hepatocytes. Use of a miR-126 target sequence may repress expression of the nucleotide sequence in endothelial cells.

In some embodiments, the interleukin is selected from the group consisting of IL-2, IL-7 and IL-15. In preferred embodiments, the interleukin is IL-2.

In preferred embodiments, the nucleotide sequence encodes IL-2 and is operably linked to one or more miR-142 target sequences and an ET promoter.

In some embodiments, the vector comprises: (a) a nucleotide sequence encoding IL-2; (b) a nucleotide sequence encoding IL-7; and/or (c) a nucleotide sequence encoding IL-15, preferably wherein each of (a)-(c) is operably linked to one or more expression control sequences for liver-specific expression.

In some embodiments, the vector is a viral vector. In some embodiments, the vector is an RNA vector, preferably an mRNA vector.

In some embodiments, the vector is a retroviral, lentiviral, adenoviral or adeno-associated viral (AAV) vector. In preferred embodiments, the vector is a lentiviral vector.

In some embodiments, the vector is an integration-defective lentiviral vector (IDLV).

In some embodiments, the vector is in the form of a viral vector particle.

In some embodiments, the viral vector particle comprises (e.g. overexpresses) CD47 (e.g. as described in U.S. Pat. No. 9,050,269). In some embodiments, the viral vector particle does not comprise or substantially does not comprise MHC-I, preferably surface-exposed MHC-I. Preferably, the viral vector particle is substantially devoid of surface-exposed MHC-I molecules. In some embodiments, the viral vector particle comprises (e.g. overexpresses) CD47 and does not comprise or substantially does not comprise MHC-I, preferably surface-exposed MHC-I.

In some embodiments, the viral vector comprises an envelope protein or capsid protein for liver cell-specific transduction. In some embodiments, the viral vector comprises an envelope protein or capsid protein for hepatocyte-specific transduction. In some embodiments, the viral vector comprises an envelope protein or capsid protein for liver sinusoidal endothelial cell-specific transduction. In some embodiments, the viral vector comprises an envelope protein or capsid protein for Kupffer cell-specific transduction.

In some embodiments, the viral vector (e.g. lentiviral vector) comprises a GP64 or hepatitis B virus envelope protein. GP64 or hepatitis B virus envelope proteins may give rise to hepatocyte-specific transduction.

In some embodiments, the vector is in the form of a liposome or lipid nanoparticle, preferably wherein the vector is an RNA vector.

In another aspect, the invention provides a composition or kit comprising two or more interleukins selected from the group consisting of: (a) the interleukin of the invention, wherein the interleukin is IL-2; (b) the interleukin of the invention, wherein the interleukin is IL-7; and (c) the interleukin of the invention, wherein the interleukin is IL-15, wherein at least two interleukins are selected from different groups (a), (b) or (c).

In another aspect, the invention provides a composition or kit comprising two or more vectors selected from the group consisting of: (a) the vector of the invention comprising a nucleotide sequence encoding IL-2; (b) the vector of the invention comprising a nucleotide sequence encoding IL-7; and (c) the vector of the invention comprising a nucleotide sequence encoding IL-15, wherein at least two vectors are selected from different groups (a), (b) or (c).

In another aspect, the invention provides a pharmaceutical composition comprising the interleukin of the invention, and a pharmaceutically-acceptable carrier, diluent or excipient.

In another aspect, the invention provides a pharmaceutical composition comprising the vector or composition of the invention, and a pharmaceutically-acceptable carrier, diluent or excipient.

In some embodiments, the pharmaceutical composition further comprises a population of T cells, preferably wherein the T cells express a chimeric antigen receptor (CAR) or a T cell receptor (TCR), which binds to a hepatitis virus antigen.

In some embodiments, the hepatitis virus antigen is selected from the group consisting of hepatitis B virus large envelope protein; hepatitis B virus middle envelope protein; hepatitis B virus small envelope protein; hepatitis B virus core protein; and hepatitis B virus polymerase.

In another aspect, the invention provides an interleukin according to the invention for use in treating or preventing a viral liver infection and/or hepatocellular carcinoma, preferably a viral liver infection.

In another aspect, the invention provides a method for treating or preventing a viral liver infection and/or hepatocellular carcinoma, comprising the step of administering the interleukin of the invention to a subject in need thereof.

In another aspect, the invention provides a vector, composition or kit according to the invention for use in treating or preventing a viral liver infection and/or hepatocellular carcinoma, preferably a viral liver infection.

In another aspect, the invention provides a method for treating or preventing a viral liver infection and/or hepatocellular carcinoma, comprising the step of administering the vector, composition or kit of the invention to a subject in need thereof.

In some embodiments, the viral liver infection is a hepatitis virus infection, preferably a chronic hepatitis virus infection.

In some embodiments, the viral liver infection is an hepatitis B virus (HBV) and/or hepatitis C virus (HCV) infection, preferably an HBV infection.

In some embodiments, the viral liver infection is a chronic hepatitis B virus (HBV) and/or a chronic hepatitis C virus (HCV) infection, preferably a chronic HBV infection.

In some embodiments, the interleukin is locally administered to a subject, preferably to a subject's liver.

In some embodiments, the interleukin is administered as part of an adoptive T cell therapy.

In some embodiments, the interleukin is administered simultaneously, separately or sequentially with a population of T cells, preferably wherein the T cells express a chimeric antigen receptor (CAR) or a T cell receptor (TCR), which binds to a hepatitis virus antigen.

In some embodiments, the vector or composition is locally administered to a subject, preferably to a subject's liver.

In some embodiments, the vector or composition is administered as part of an adoptive T cell therapy.

In some embodiments, the vector is administered simultaneously, separately or sequentially with a population of T cells, preferably wherein the T cells express a chimeric antigen receptor (CAR) or a T cell receptor (TCR), which binds to a hepatitis virus antigen.

In some embodiments, the hepatitis virus antigen is selected from the group consisting of hepatitis B virus large envelope protein; hepatitis B virus middle envelope protein; hepatitis B virus small envelope protein; hepatitis B virus core protein; and hepatitis B virus polymerase.

In another aspect, the invention provides an interleukin according to the invention for use in increasing effector responses in T cells, preferably CD8+ T cells.

In another aspect, the invention provides a method for increasing effector responses in T cells, preferably CD8+ T cells, comprising the step of administering the interleukin of the invention to a subject in need thereof.

In another aspect, the invention provides a vector, composition or kit according to the invention for use in increasing effector responses in T cells, preferably CD8+ T cells.

In another aspect, the invention provides a method for increasing effector responses in T cells, preferably CD8+ T cells, comprising the step of administering the vector, composition or kit of the invention to a subject in need thereof.

In some embodiments, the effector responses are against hepatotropic viruses, such as hepatitis virus, preferably HBV.

In some embodiments, the T cells are dysfunctional T cells. In some embodiments, the T cells are from an immune-tolerant subject.

In another aspect, the invention provides an interleukin according to the invention for use in increasing T cell antiviral activity, preferably CD8+ T cell antiviral activity.

In another aspect, the invention provides a method for increasing T cell antiviral activity, preferably CD8+ T cell antiviral activity, comprising the step of administering the interleukin of the invention to a subject in need thereof.

In another aspect, the invention provides a vector, composition or kit according to the invention for use in increasing T cell antiviral activity, preferably CD8+ T cell antiviral activity.

In another aspect, the invention provides a method for increasing T cell antiviral activity, preferably CD8+ T cell antiviral activity, comprising the step of administering the vector, composition or kit of the invention to a subject in need thereof.

In some embodiments, the antiviral activity is against hepatotropic viruses, such as hepatitis virus, preferably HBV.

In another aspect, the invention provides a product comprising (a) the interleukin of the invention; and (b) a population of T cells, as a combined preparation for simultaneous, separate or sequential use in therapy, preferably wherein the T cells express a chimeric antigen receptor (CAR) or a T cell receptor (TCR), which binds to a hepatitis virus antigen.

In another aspect, the invention provides a product comprising (a) the vector or composition of the invention; and (b) a population of T cells, as a combined preparation for simultaneous, separate or sequential use in therapy, preferably wherein the T cells express a chimeric antigen receptor (CAR) or a T cell receptor (TCR), which binds to a hepatitis virus antigen.

In some embodiments, the product is for use in treating or preventing a viral liver infection or hepatocellular carcinoma, preferably a viral liver infection.

In some embodiments, the viral liver infection is a hepatitis virus infection, preferably a chronic hepatitis virus infection.

In some embodiments, the viral liver infection is an hepatitis B virus (HBV) and/or hepatitis C virus (HCV) infection, preferably an HBV infection.

In some embodiments, the viral liver infection is a chronic hepatitis B virus (HBV) and/or a chronic hepatitis C virus (HCV) infection, preferably a chronic HBV infection.

In some embodiments, the hepatitis virus antigen is selected from the group consisting of hepatitis B virus large envelope protein; hepatitis B virus middle envelope protein; hepatitis B virus small envelope protein; hepatitis B virus core protein; and hepatitis B virus polymerase.

In some embodiments, the interleukin and/or population of T cells is locally administered to a subject, preferably to a subject's liver.

In some embodiments, the vector and/or population of T cells is locally administered to a subject, preferably to a subject's liver.

### DESCRIPTION OF THE DRAWINGS

FIG. 1

Naïve CD8+ T Cells that Recognize Hepatocellular Ag Expand but Fail to Develop Effector Functions.

(A) Schematic representation of the experimental setup. 5×10⁶ Env28 T_{N} were transferred into C57BL/6×Balb/c F1 (WT) or HBV replication-competent transgenic (HBV Tg, C57BL/6×Balb/c F1) recipients. Livers were collected and analyzed five days after Env28 IN transfer and sera from the same mice were collected every day from day 0 to day 5 after Env28 T_{N} transfer. (B-C) Absolute numbers (B) and frequency of IFNγ-producing (C) Env28 T cells in the livers of the indicated mice. (D) ALT levels detected in the sera of the indicated mice at the indicated time points. Data are representative of at least 3 independent experiments. *** p value <0.001

FIG. 2

Spatiotemporal Dynamics of Naïve CD8+ T Cells Undergoing Intrahepatic Priming.

(A) Schematic representation of the experimental setup. 5×10⁶ naïve CD8+ T cells from Cor93 TCR transgenic mice (Cor93 T_{N}) were transferred into C57BL/6 (WT) or MUP-core recipients. Mice were splenectomized and treated with anti-CD62L Abs 48 hours and 4 hours prior to cell transfer, respectively. When indicated, mice were injected with 2.5×10⁵ infectious units of non-replicating rLCMV-core 4 hours prior CD8+ T cell transfer. Livers were collected and analyzed at the indicated time points. n=4 (WT), 7 (WT+rLCMV-core and MUP-core) (B-C) Absolute numbers (B) and frequency of IFN-γ-producing (C) Cor93 T cells in the livers of indicated mice at the indicated time points. (D) Mean Fluorescence Intensity (MFI) of PD-1 expression on Cor93 T cells in the livers of indicated mice. (E) (Left panels) Representative confocal immunofluorescence micrographs of liver sections from WT+rLCMV-core (upper panels) or from MUP-core mice (lower panels) three days after Cor93 T_{N} transfer. Distribution of Cor93 T cells (green) relative to portal tracts (highlighted by anti-cytokeratin 7 Ab-mediated staining of bile ducts in red). Sinusoids are highlighted by anti-Lyve-1⁺ Abs (white). Scale bars represent 100 μm. (Middle panels) Hematoxylin and eosin staining of liver sections from the same mice, where dotted lines denote leukocyte clusters. Scale bars represent 300 μm. (Right panels) Snapshots from representative intravital multiphoton microscopy movies of the same mice. Cor93 T cells tracks (yellow lines) were recorded during an hour movie. Blood vessels are depicted in white. Scale bars represent 40 μm. See also FIG. 5. (F) Mean speed of Cor93 T cells in the livers of the indicated mice. (G) 5×10⁶ naïve CD8+ T cells from Cor93 and Env28 TCR transgenic mice were co-transferred into C57BL/6×Balb/c F1 (WT) or MUP-core×Balb/c F1 (MUP-core) recipients. Mice were splenectomized and treated with anti-CD62L 48 hours and 4 hours prior to cell transfer, respectively. When indicated, mice were injected with 2.5×10⁵ infectious units of non-replicating rLCMV-env or rLCMV-core/env 4 hours prior to CD8+ T cell transfer. Livers were collected and analyzed by flow cytometry five days after T cell transfer. Total numbers (left) and numbers of IFN-γ-producing (right) Cor93 and Env28 T cells in the livers of the indicated mice. Data are representative of at least 3 independent experiments. * p value <0.05, ** p value <0.01, *** p value <0.001

FIG. 3

Ag Recognition by Naïve CD8+ T Cells in MUP-Core or rLCMV-Core-Injected WT Mice is Restricted to the Liver Upon Splenectomy and Anti-CD62L Ab Treatment.

(A-C) 5×10⁶ Cor93 T_{N} were transferred into C57BL/6 (WT) or MUP-core recipients. Mice were splenectomized and treated with anti-CD62L 48 hours and 4 hours prior to cell transfer, respectively. Untreated WT mice that received 5×10⁶ Cor93 T_{N} were used as controls. Where indicated, mice were injected with 2.5×10⁵ infectious units of non-replicating rLCMV-core 4 hours prior to Cor93 T_{N} transfer. Liver-draining lymph nodes (Barbier, L. et al., 2012. J Hepatol 57, 352-358) (dLN) and non-draining inguinal lymph nodes (ndLN) were collected at four hours and one day after Cor93 T_{N}. A representative flow cytometry plot at four hours upon Cor93 T_{N} transfer is shown in A. Numbers indicate the percentage of cells within the indicated gate. (B-C) Quantification of the absolute numbers of cells recovered from the ndLN (B) and dLN (C) of the indicated mice four hours and one day upon Cor93 T_{N} transfer. n=3. (D) Confocal immunofluorescence micrographs of liver sections from WT mice (WT), rLCMV-core-injected WT mice (WT+rLCMV-core), MUP-core mice and R26-ZsGreen mice injected with 2.5×10⁵ infectious units of non-replicating rLCMV-cre (R26-ZsGreen+rLCMV-cre). Scale bars represent 100 μm. Note that, because HBV core protein did not accumulate at detectable levels in KCs and hepatic dendritic cells [DCs] upon rLCMV-core injection, we confirmed the tropism of this vector by injecting rLCMV-cre into R26-ZsGreen mice—these mice express the fluorescent protein ZsGreen upon Cre-mediated recombination. (E) Mean.

Fluorescent Intensity (MFI) of CD69 expression on Cor93 T cells in the liver, blood, lung and bone marrow of the indicated mice four hours after Cor93 T_{N} transfer. n=4. Data are representative of at least 3 independent experiments. *** p value <0.001

FIG. 4

CD8+ T Cell Effector Differentiation Upon Intrahepatic Priming is Independent of the Number of Adoptively Transferred T_{N}.

2×10⁶, 2×10⁵ or 2×10⁴ Cor93 T_{N} were transferred into C57BL/6 (WT) or MUP-core recipients. Mice were splenectomized and treated with anti-CD62L 48 hours and 4 hours prior to cell transfer, respectively. Where indicated, mice were injected with 2.5×10⁵ infectious units of non-replicating rLCMV-core 4 hours prior to Cor93 T_{N} transfer. Absolute numbers of Cor93 T cells recovered from the livers of the indicated mice 5 days after transfer are shown. Data are representative of 2 independent experiments.

FIG. 5

Spatiotemporal Dynamics of Naïve CD8+ T Cells Upon Intrahepatic Priming.

5×10⁶ fluorescent Cor93 T_{N} were transferred into MUP-core or rLCMV-core-injected WT recipients. Mice were splenectomized and treated with anti-CD62L 48 hours and 4 hours prior to Cor93 T_{N} transfer, respectively. (A) (left panels) Confocal immunofluorescence micrographs of liver sections from the indicated mice at the indicated timepoints upon Cor93 T_{N} transfer, showing the distribution of Cor93 T cells (green) relative to portal tracts (highlighted by anti-cytokeratin 7 Ab-mediated staining of bile ducts in red). Sinusoids are highlighted by anti-Lyve-1⁺ Abs (white). Scale bars represent 100 μm. (right panels) Immunohistochemical micrographs of liver sections from the indicated mice at the indicated timepoints upon Cor93 T_{N} transfer, showing the distribution of leukocyte infiltrates relative to portal tracts (highlighted by anti-cytokeratin 7 Ab-mediated staining of bile ducts in brown). Scale bars represent 100 μm. (B) Distribution of the distances (μm) of each Cor93 T cell from the center of the closest portal triad at the indicated timepoints. n=3. Data are representative of at least 3 independent experiments.

FIG. 6

CD4+ T Cells are not Required for CD8+ T Cell Effector Differentiation Upon Intrahepatic Priming.

(A) Schematic representation of the experimental setup. 5×10⁶ Cor93 T_{N} were transferred to C57BL/6 (WT) recipients. Mice were splenectomized and treated with anti-CD62L 48 hours and 4 hours prior to Cor93 T_{N} transfer, respectively, and injected with 2.5×10⁵ infectious units of non-replicating rLCMV-core 4 hours prior to Cor93 T_{N} transfer. Where indicated, mice were injected with anti-CD4 depleting antibodies 72 and 24 hours prior to Cor93 T_{N} transfer. (B) CD4+ T cells in peripheral blood (indicated as percentage of total CD3⁺ T cells) of the indicated mice were analyzed 24 hours after the second injection of anti-CD4 depleting antibodies. (C-D) Absolute numbers (C) and numbers of IFN-γ producing (D) Cor93 T cells in the livers of the indicated mice five days after Cor93 T_{N} transfer. (E) Confocal immunofluorescence micrographs of liver sections from the indicated mice five days after Cor93 T_{N} transfer. Yellow dotted lines denote T cell clusters. Scale bars represent 100 μm. Data are representative of 2 independent experiments.

FIG. 7

Kupffer Cells are Required for CD8+ T Cell Effector Differentiation Upon Intrahepatic Priming.

(A) Schematic representation of the experimental setup. 5×10⁶ Cor93 T_{N} were transferred into C57BL/6 (WT) recipients. Mice were splenectomized and treated with anti-CD62L 48 hours and 4 hours prior to cell transfer, respectively and injected with 2.5×10⁵ infectious units of non-replicating rLCMV-core 4 hours prior to Cor93 T_{N} transfer. Where indicated, mice were treated with clodronate liposomes (CLL) 48 hours prior to Cor93 T_{N} transfer. (B) Confocal microscopy of liver sections from control mice (left panels) and clodronate liposomes-treated mice (right panels). Kupffer cells are depicted in red in all panels, while sinusoids are depicted in grey only in the first and third panel. Scale bars represent 100 □m. (C-D) Percentages (C) and absolute numbers (D) of CD11c⁺ MHC-II^{high} dendritic cells in the livers of the indicated mice. (E-F) Absolute numbers (E) and numbers of IFN-□ producing (F) Cor93 T cells in the livers of the indicated mice five days after Cor93 T_{N} transfer. (G) Confocal immunofluorescence micrographs of liver sections from the indicated mice five days after Cor93 T_{N} transfer. Scale bars represent 100 μm. Data are representative of 3 independent experiments.

FIG. 8

The CD8+ T Cell Dysfunction Induced by Hepatocellular Priming does not Depend on CD4+ Regulatory T Cells.

(A) Schematic representation of the experimental setup. 5×10⁶ Cor93 T_{N} were transferred into MUP-core recipients. Mice were splenectomized and treated with anti-CD62L 48 hours and 4 hours prior to cell transfer, respectively. Where indicated, mice were injected with anti-CD4 depleting antibodies 72 and 24 hours prior to Cor93 T_{N} transfer. Livers were collected and analyzed five days after Cor93 T_{N} transfer. (B) Percentage of CD4⁺ T cells (out of total CD3⁺ T cells) in the peripheral blood of the indicated mice were analyzed 24 hours after the second injection of anti-CD4 depleting antibodies. (C-D) Absolute numbers (C) and numbers of IFN-γ-producing (D) Cor93 T cells in the livers of the indicated mice five days after Cor93 T_{N} transfer. (E) Confocal immunofluorescence micrographs of liver sections from the indicated mice five days after Cor93 T_{N} transfer. Cor93 T cells are depicted in green and sinusoids in grey. Scale bars represent 100 μm. Data are representative of 2 independent experiments.

FIG. 9

CD8+ T Cell Dysfunction Induced by Hepatocellular Priming is not Dependent on the Number of Ag-Presenting Hepatocytes.

(A) Schematic representation of the experimental setup. 5×10⁶ Cor93 T_{N} were transferred into C57BL/6 (WT) or MUP-core recipients. Mice were splenectomized and treated with anti-CD62L 48 hours and 4 hours prior to Cor93 T_{N} transfer, respectively. Indicated WT mice were injected with 3×10¹⁰ viral genomes of AAV-core or with 2.5×10⁵ infectious units of non-replicating rLCMV-core 15 days or 4 hours prior to Cor93 T_{N} transfer, respectively. Livers were collected and analyzed five days after Cor93 T_{N} transfer. (B-C) Absolute numbers (B) and percentage of IFN-γ-producing (C) Cor93 T cells in the livers of indicated mice five days after Cor93 T_{N} transfer. (D) Confocal immunofluorescence micrographs of liver sections from the indicated mice five days after Cor93 T_{N} transfer. Cor93 T cells are depicted in green and sinusoids in grey. Scale bars represent 100 μm. Data are representative of 2 independent experiments.

FIG. 10

Transcriptomic and Chromatin Accessibility Analyses of CD8+ T Cells Undergoing Intrahepatic Priming.

(A-D) 5×10⁶ Cor93 T_{N} were transferred into WT or MUP-core recipients. Mice were splenectomized and treated with anti-CD62L 48 hours and 4 hours prior to cell transfer, respectively. Where indicated, mice were injected with 2.5×10⁵ infectious units of non-replicating rLCMV-core 4 hours prior to Cor93 T_{N} transfer. Livers were collected at days 1, 3 and 7 after T cell transfer and Cor93 T cells were FACS-sorted prior to RNA-seq and ATAC-seq analyses. Cor93 T cell purity was always greater than 98% (data not shown). (A) Scatter plot showing the behavior of inducible genes in the dataset (log fold-change [FC]>2.5, false discovery rate [FDR]<0.01 versus Cor93 T_{N}). The y axis indicates transcript levels in the indicated samples and the x axis indicates the differential gene expression (log FC) between Cor93 T cells from WT+rLCMV-core and from MUP-core mice. Genes expressed at higher levels in Cor93 T cells from WT+rLCMV-core (log FC>1.5, FDR<0.1) or from MUP-core mice (log FC<−1.5, FDR<0.1) are shown in blue and red, respectively. Genes expressed at similar levels in the two conditions are shown in grey. Selected representative genes are highlighted by arrows. (B) Integrative Genome Viewer (IGV) (Robinson, J. T. et al., 2011. Nat Biotechnol 29, 24-26) snapshots showing RNA-seq and ATAC-seq data at Gzmk and Areg loci, selected as representative genes with differential expression in Cor93 T cells from WT+rLCMV-core and from MUP-core mice. Data from individual biological replicates are merged for visualization purposes. (C) Bar plot showing the number of inducible ATAC-seq peaks (log FC>2.5, FDR<0.001 versus Cor93 T_{N}) in the indicated conditions. ATAC-seq peaks with higher intensity signal in Cor93 T cells from WT+rLCMV-core (log FC>1.5, FDR<0.1) and from MUP-core mice (log FC<−1.5, FDR<0.1) are shown in blue and red, respectively. (D) Motif enrichment (HOMER) (Heinz, S. et al., 2010. Molecular Cell 38, 576-589) analysis of the top 200 differential ATAC-seq peaks identified at day 7 in Cor93 T cells from WT+rLCMV-core (top) or MUP-core (bottom) mice, as compared to 3899 non-inducible ATAC-seq peaks. Values between brackets indicate p value. (E) Schematic representation of the experimental setup. 5×10⁶ Cor93 T_{N} were transferred into WT or MUP-core recipients. Mice were splenectomized and treated with anti-CD62L 48 hours and 4 hours prior to cell transfer, respectively. Where indicated, mice were injected with 2.5×10⁵ infectious units of non-replicating rLCMV-core 4 hours prior to Cor93 T_{N} transfer. Livers were collected either 4 hours or three days after cell transfer. 5×10³ purified Cor93 T cells were injected back into rLCMV-core-injected WT mice (which were splenectomized and treated with anti-CD62L as described previously). Livers were collected and analyzed by flow cytometry five days after Cor93 T cell transfer. (F-G) Total numbers (F) and numbers of IFN-γ-producing (G) Cor93 T cells in the livers of the indicated mice. Data are representative of at least 3 experiments. ** p value <0.01.

FIG. 11

Correlation Heatmap of RNA-Seq Datasets.

Numbers indicate Pearson's correlation values.

FIG. 12

Mean Expression Levels of Genes Differentially Expressed in Naïve CD8+ T Cells Undergoing Intrahepatic Priming, Related to FIG. 10A.

(A-C) Box plots showing expression levels (log 2RPKM) in the indicated experimental condition of genes belonging to the categories described in FIG. 10A. Results of Mann-Whitney U test are shown for the indicated comparisons.

FIG. 13

Correlation Heatmap of ATAC-Seq Datasets.

Numbers indicate Pearson's correlation values.

FIG. 14

Mean Signal Intensity of ATAC-Seq Peaks in Naïve CD8+ T Cells Undergoing Intrahepatic Priming, Related to FIG. 10C.

(A-C) Box plots showing ATAC-seq signal intensity (log₂CPM) in the indicated experimental condition of peaks belonging to the categories described in FIG. 10C. Results of Mann-Whitney U test are shown for the indicated comparisons.

FIG. 15

Intrahepatically-Primed, Dysfunctional CD8+ T Cells can be Rescued by IL-2, but not by Anti-PD-L1 Abs.

(A) Heatmaps showing the enrichment of gene ontology (GO) categories within genes expressed at higher levels in Cor93 T cells from WT+rLCMV-core (left) or MUP-core (right) mice at the indicated time points. GO categories were identified by gene set enrichment analysis (GSEA) (Subramanian, A. et al., 2005. Proc Natl Acad Sci USA 102, 15545-15550) and then grouped by similarity using REVIGO (Supek, F., et al., 2011. PLoS ONE 6, e21800). Selected representative GO categories are indicated in the figure. Colors represent Normalized Enrichment Score (NES) values. (B) IGV snapshots showing RNA-seq and ATAC-seq data at Pdcd1 and 112 loci. (C) Schematic representation of the experimental setup. 5×10⁶ Cor93 T_{N} were transferred into C57BL/6 WT or MUP-core recipients. Mice were splenectomized and treated with anti-CD62L 48 hours and 4 hours prior to Cor93 T_{N} transfer, respectively. Where indicated, mice were injected with 2.5×10⁵ infectious units of non-replicating rLCMV-core 4 hours prior to Cor93 T_{N} transfer. MUP-core mice received anti-PD-L1 Abs and/or IL-2/anti-IL-2 complexes (IL2c) at the indicated time-points. Livers and sera were collected and analyzed at d ay 5 after Cor93 T_{N} transfer. (D-E) Total numbers (D) and numbers of IFN-γ-producing (E) Cor93 T cells in the livers of the indicated mice. (F) ALT levels in the sera of the indicated mice. (G) Percentage of genes with lower (left bar) or higher (right bar) expression at day 5 in Cor93 T cells from MUP-core mice compared to Cor93 T cells from rLCMV-core-injected WT mice. Colors discriminate the percentage of genes that are rescued (dark grey, log FC>1.5 for hypo-expressed genes, log FC<−1.5 for hyper-expressed genes), partially rescued (light grey, 1.5<log FC<1 for hypo-expressed genes, −1.5<log FC<−1 for hyper-expressed genes) or not rescued (white, log FC<1 for hypo-expressed genes, log FC>-1 for hyper-expressed genes) by IL-2c administration. (H) Box plots showing expression levels in the indicated experimental condition of genes hypo-expressed (left plot) or hyper-expressed (right) at day 5 in Cor93 T cells from MUP-core mice compared to Cor93 T cells from rLCMV-core-injected WT mice. Data are representative of at least 3 independent experiments. ** p value <0.01, *** p value <0.001

FIG. 16

The Transcriptional Program of Intrahepatically Primed CD8+ T Cells does not Obviously Overlap with that of Splenic LCMV-Specific Exhausted CD8+ T Cells

(A) Heatmap showing expression values (log 2RPKM) in RNA-seq data from splenic LCMV-specific effector or exhausted CD8⁺ T cells (Scott-Browne, J. P. et al., 2016. Immunity 45, 1327-1340) of the top 100 most differential genes in Cor93 T cells isolated from MUP-core livers. (B) Number of top 100 genes from Cor93 T cells recovered from MUP-core mice reaching log₂RPKM>1 in the indicated conditions in RNA-seq data from splenic LCMV-specific effector or exhausted CD8⁺ T cells (Scott-Browne, J. P. et al., 2016. Immunity 45, 1327-1340). (C) Box plot showing the expression levels of top 100 genes from Cor93 T cells recovered from MUP-core mice in the indicated conditions in RNA-seq data from Scott-Browne, J. P. et al., 2016. Immunity 45, 1327-1340 [Wilcoxon Rank Sum Test: (*) p-value≤0.05; (**) p-value ≤0.01; (***) p-value ≤0.001; (n.s.) Not Significant].

FIG. 17

The Transcriptional Program of Intrahepatically Primed CD8+ T Cells does not Obviously Overlap with that of Splenic LCMV-Specific Exhausted CD8+ T Cells

(A) Heatmap showing expression values (log 2RPKM) in RNA-seq data from splenic LCMV-specific effector or exhausted CD8⁺ T cells (Scott-Browne, J. P. et al., 2016. Immunity 45, 1327-1340) of the top 100 most differential genes in Cor93 T cells isolated from WT+rLCMV-core livers. (B) Number of top 100 genes from Cor93 T cells recovered from WT+rLCMV-core livers reaching log₂RPKM>1 in the indicated conditions in RNA-seq data from splenic LCMV-specific effector or exhausted CD8⁺ T cells (Scott-Browne, J. P. et al., 2016. Immunity 45, 1327-1340). (C) Box plot showing the expression levels of top 100 genes from Cor93 T cells recovered from WT+rLCMV-core livers in the indicated conditions in RNA-seq data from splenic LCMV-specific effector or exhausted CD8⁺ T cells (Scott-Browne, J. P. et al., 2016. Immunity 45, 1327-1340) [Wilcoxon Rank Sum Test: (*) p-value ≤0.05; (**) p-value ≤0.01; (***) p-value ≤0.001; (n.s.) Not Significant].

FIG. 18

The Transcriptional Program of Intrahepatically Primed CD8+ T Cells does not Obviously Overlap with that of Splenic LCMV-Specific Exhausted CD8+ T Cells

(A) Heatmap showing expression values (log 2RPKM) in RNA-seq data from splenic LCMV-specific exhausted CD8⁺ T cells (from Pauken, K. E. et al., 2016. Science 354, aaf2807-1165) of the top 100 most differential genes in Cor93 T cells isolated from MUP-core livers. (B) Number of top 100 genes from Cor93 T cells recovered from MUP-core mice reaching log₂RPKM>1 in the indicated conditions in RNA-seq data from splenic LCMV-specific exhausted CD8⁺ T cells (from Pauken, K. E. et al., 2016. Science 354, aaf2807-1165). (C) Box plot showing the expression levels of top 100 genes from Cor93 T cells recovered from MUP-core mice in the indicated conditions in RNA-seq data from splenic LCMV-specific exhausted CD8⁺ T cells (from Pauken, K. E. et al., 2016. Science 354, aaf2807-1165). [Wilcoxon Rank Sum Test: (*) p-value ≤0.05; (**) p-value ≤0.01; (***) p-value ≤0.001; (n.s.) Not Significant].

FIG. 19

The Transcriptional Program of Intrahepatically Primed CD8+ T Cells does not Obviously Overlap with that of Splenic LCMV-Specific Exhausted CD8+ T Cells

(A) Heatmap showing expression values (log 2RPKM) in RNA-seq data from splenic LCMV-specific exhausted CD8⁺ T cells (from Pauken, K. E. et al., 2016. Science 354, aaf2807-1165) of the top 100 most differential genes in Cor93 T cells isolated from WT+rLCMV-core livers. (B) Number of top 100 genes in Cor93 T cells isolated from WT+rLCMV-core livers reaching log₂RPKM>1 in the indicated conditions in RNA-seq data from splenic LCMV-specific exhausted CD8⁺ T cells (from Pauken, K. E. et al., 2016. Science 354, aaf2807-1165). (C) Box plot showing the expression levels of top 100 genes in Cor93 T cells isolated from WT+rLCMV-core livers in the indicated conditions in RNA-seq data from splenic LCMV-specific exhausted CD8⁺ T cells (from Pauken, K. E. et al., 2016. Science 354, aaf2807-1165) [Wilcoxon Rank Sum Test: (*) p-value ≤0.05; (**) p-value ≤0.01; (***) p-value ≤0.001; (n.s.) Not Significant].

FIG. 20

The Transcriptional Program of Intrahepatically Primed CD8+ T Cells does not Obviously Overlap with that of Tolerant Self-Ag-Specific CD8+ T Cells

(A) Heatmap showing normalized expression values in microarray data from tolerant self-Ag-specific CD8⁺ T cells (from Schietinger, A., et al., 2012. Science 335, 723-727) of genes retrieved in the dataset among the top 100 most differential genes in Cor93 T cells isolated from MUP-core livers. (B) Number of top 100 genes from Cor93 T cells recovered from MUP-core mice expressed (log 2(normalized data) >65th percentile of the full distribution) in the indicated conditions in microarray data from tolerant self-Ag-specific CD8⁺ T cells (from Schietinger, A., et al., 2012. Science 335, 723-727). (C) Box plot showing the expression levels of genes retrieved in the dataset among the top 100 genes from Cor93 T cells recovered from MUP-core mice in the indicated conditions in microarray data from tolerant self-Ag-specific CD8⁺ T cells (from Schietinger, A., et al., 2012. Science 335, 723-727). Only genes for which microarray probes were retrieved were kept for these analyses. [Wilcoxon Rank Sum Test: (*) p-value ≤0.05; (**) p-value ≤0.01; (***) p-value ≤0.001; (n.s.) Not Significant].

FIG. 21

The Transcriptional Program of Intrahepatically Primed CD8+ T Cells does not Obviously Overlap with that of Tolerant Self-Ag-Specific CD8+ T Cells

(A) Heatmap showing normalized expression values in microarray data from tolerant self-Ag-specific CD8⁺ T cells (from Schietinger, A., et al., 2012. Science 335, 723-727) of genes retrieved in the dataset among the top 100 most differential genes in Cor93 T cells isolated from WT+rLCMV-core livers. (B) Number of top 100 genes in Cor93 T cells isolated from WT+rLCMV-core livers expressed (log 2(normalized data) >65th percentile of the full distribution) in the indicated conditions in microarray data from tolerant self-Ag-specific CD8⁺ T cells (from Schietinger, A., et al., 2012. Science 335, 723-727). (C) Box plot showing the expression levels of genes retrieved in the dataset among the top 100 genes in Cor93 T cells isolated from WT+rLCMV-core livers in the indicated conditions in microarray data from tolerant self-Ag-specific CD8⁺ T cells (from Schietinger, A., et al., 2012. Science 335, 723-727). Only genes for which microarray probes were retrieved were kept for these analyses. [Wilcoxon Rank Sum Test: (*) p-value ≤0.05; (**) p-value ≤0.01; (***) p-value ≤0.001; (n.s.) Not Significant].

FIG. 22

IL-2 is Induced in the Livers of rLCMV-Core-Transduced WT Mice.

IL-2 gene expression (normalized to the reference gene GAPDH) was measured by quantitative PCR in the livers of the indicated mice. Data are representative of at least 3 independent experiments. ** p value <0.01, *** p value <0.001

FIG. 23

IL-2 Substantially Rescues the Transcriptional Program of Dysfunctional CD8+ T Cells.

(A-B) Heatmap showing expression values (log 2RPKM) of hypo-expressed (A) or hyper-expressed (B) genes in Cor93 CD8⁺ T cells from MUP-core livers at day 5, which are rescued by IL-2c. Data refer to genes identified in FIG. 15G-H.

FIG. 24

Therapeutic Reinvigoration of Intrahepatically-Primed, Dysfunctional CD8+ T Cells by IL-2 Requires KC Cross-Presentation of Hepatocellular Ags.

(A) Schematic representation of the experimental setup. 5×10⁶ Cor93 and Env28 T_{N} were transferred into C57BL/6×Balb/c F1 (WT) or MUP-core×Balb/c F1 (MUP-core) recipients. Mice were splenectomized and treated with anti-CD62L 48 hours and 4 hours prior to T_{N} transfer, respectively. When indicated, mice were injected with 2.5×10⁵ infectious units of non-replicating rLCMV-core/env 4 hours prior to T_{N} transfer. Selected MUP-core mice received clodronate liposomes (CLL) for Kupffer cell depletion and/or IL2c at the indicated time-points. Livers were collected and analyzed five days after T_{N} transfer. (B-C) Total numbers (B) and numbers of IFN-γ-producing (C) T cells in the livers of indicated mice are shown. (D) Representative confocal immunofluorescence micrographs of liver sections from the indicated mice five days after T_{N} transfer. Cor93 T cells are depicted in red, Env28 T cells in red and sinusoids in grey. Scale bars represent 100 μm. (E) Schematic representation of the experimental setup. MUP-core mice were lethally irradiated and reconstituted with WT or TAP1^{-/-} bone marrow (BM). Six weeks later mice received two injection of clodronate liposomes (CLL) to remove residual radio-resistant KCs and allow full reconstitution of KCs from donor-derived BM. Eight weeks after BM reconstitution, 5×10⁶ Cor93 T_{N} were transferred. Mice were splenectomized and treated with anti-CD62L 48 hours and 4 hours prior to T_{N} transfer, respectively. Indicated mice received IL2c at the indicated time-points. Livers were collected and analyzed five days after T_{N} transfer. (F-G) Total numbers (F) and numbers of IFN-γ-producing (G) T cells in the livers of the indicated mice. Data are representative of at least 3 independent experiments. ** p value <0.01, *** p value <0.001

FIG. 25

Flow Cytometry Analysis of IL-2Rα (CD25) Expression on Kupffer Cells.

Kupffer cells (KCs) were defined as CD45⁺Lin⁻CD64⁺F4/80⁺TIM4⁺ cells among the live singlets. The percentage of CD25⁺ cells among KCs is shown on the bottom right (each dot represents an individual mouse). Data are representative of 3 independent experiments.

FIG. 26

Therapeutic Reinvigoration of Intrahepatically-Primed, Dysfunctional CD8+ T Cells by IL-2.

(A) Schematic representation of the experimental setup. 5×10⁶ Cor93 T_{N} were transferred into HBV replication-competent transgenic (HBV Tg) recipients. Indicated HBV Tg mice received IL2c treatment one day after CD8⁺ T cell transfer. Livers were collected and analyzed five days after Cor93 T_{N} transfer and sera from the same mice were collected at day 5 after Cor93 T_{N} transfer. (B) Absolute numbers of IFN-□-producing Cor93 T cells in the livers of the indicated mice. (C) ALT levels detected in the sera of the indicated mice. (D) Confocal immunofluorescence micrographs of liver sections from the indicated mice five days after Cor93 T_{N} transfer. Cor93 T cells are depicted in green and sinusoids in grey. Scale bars represent 100 μm. Data are representative of at least 3 independent experiments. *** p value <0.001

FIG. 27

Therapeutic Potential of IL-2 Treatment for T Cell Reinvigoration During Chronic HBV Infection.

(A-D) T cells from 13 Immune Tolerant (IT) and 16 Immune Active (IA) chronic HBV patients were stimulated with genotype-specific overlapping HBV peptides that cover the entire HBV proteome and cultured for 10 days in the presence or absence (NT) of recombinant human IL-2. Subsequently, T cells where re-stimulated with the HBV peptide pools and the frequency of HBV-specific T cells was determined as spot forming units (SFU) by IFN-γ ELISpot assay. Number of HBV-specific T cells from IT (A) and IA patients (B) cultured with or without 20 IU/mL of IL-2 are shown. The percentage of IT (C) and IA patients (D) for which the HBV-specific T cell expansion increased by more than 2-fold with addition of IL-2 during the culture are shown in black. (E) Schematic representation of the experimental setup. 5×10⁶ Cor93 and TCR-I (control) T_{N} were transferred into C57BL/6 WT or MUP-core recipients. Mice were splenectomized and treated with anti-CD62L 48 hours and 4 hours prior to cell transfer, respectively. Where indicated, mice were injected with 2.5×10⁸ (low) or 5×10⁸ (high) transforming units of LV.ET.mIL2.142T (LV-IL2, see FIG. 25) seven days prior to T_{N} transfer. Livers were collected and analyzed five days after T_{N} transfer. (F) Absolute numbers of IFN-γ-producing T cells in the livers of the indicated mice are shown. (G) Sera from the same mice were collected at day 0 and day 5. ALT levels detected in the sera of indicated mice are shown. (H) IL-2 gene expression (normalized to the reference gene GAPDH) was measured by quantitative PCR in the livers of the indicated mice. Data are representative of at least 3 independent experiments. ** p value <0.01, *** p value <0.001

FIG. 28

Lentiviral-Mediated Hepatic Expression of IL-2 Reinvigorates Intrahepatically Primed, Dysfunctional CD8+ T Cells.

(A) Schematic representation of the third-generation self-inactivating (SIN) lentivirus (proviral form) used in this work. SIN LTR: SIN HIV Long Terminal Repeat (LTR) with deletion of the U3 promoter/enhancer region (Zufferey, R. et al., 1998. J Virol 72, 9873-9880); packaging signal; hepatocyte-specific Enhanced Transthyretin (ET) promoter composed of synthetic hepatocyte-specific enhancers and transthyretin promoter (Vigna, E. et al., 2005. Mol. Ther. 11, 763-775); 142-T: microRNA 142 target sequence made of 4 tandem copies of a sequence perfectly complementary to microRNA 142; WPRE: woodchuck hepatitis virus post-transcriptional regulatory element. The cDNA of murine interleukin 2 (mIL-2) was used as transgene. (B) 5×10⁶ Cor93 T_{N} were transferred into MUP-core recipients. Mice were splenectomized and treated with anti-CD62L 48 hours and 4 hours prior to cell transfer, respectively. Where indicated, mice were injected with 10⁹ transforming units of integrase-defective LV.ET.mIL2.142T (IDLV-IL2) seven days prior to T_{N} transfer. ALT were measured in the sera of the indicated mice five days after T_{N} transfer. Data are representative of two independent experiments. *** p value <0.001

FIG. 29

IL-2c Treatment Reduces HBV Replication in Transgenic Mice.

(A) Schematic representation of the experimental setup. 1×10⁶ Cor93 T_{N} were transferred into HBV replication-competent transgenic (HBV Tg) recipients. Indicated HBV Tg mice received IL-2c treatment one day after CD8⁺ T cell transfer. Livers were collected and analysed five days after Cor93 T_{N} transfer and sera from the same mice were collected prior to and five days after Cor93 T_{N}. (B) Serum HBV DNA in control and IL-2c-treated HBV Tg mice was measured by qPCR. (C) Hepatic HBV replicative DNA intermediated in control and IL-2c-treated HBV Tg mice was assessed by Southern Blot analysis. (D) Immunohistochemical micrographs of liver sections from the indicated mice, showing core Ag expression (highlighted by anti-HBcAg Ab-mediated staining in brown). Scale bars represent 100 μm. Data are representative of at least 3 independent experiments. ** p value <0.01, *** p value <0.001

FIG. 30

Therapeutic Restoration of Intrahepatically-Primed, Dysfunctional CD8+ T Cells by IL-2 Requires KC Cross-Presentation of Hepatocellular Ags.

(A) Schematic representation of the experimental setup. 5×10⁶ Cor93 and Env28 T_{N} were transferred into C57BL/6×Balb/c F1 (WT) or MUP-core×Balb/c F1 (MUP-core) recipients. When indicated, mice were injected with 2.5×10⁵ infectious units of non-replicating rLCMV-core/env 4 hours prior prior to T_{N} transfer. Selected MUP-core mice received clodronate liposomes (CLL) for Kupffer cell depletion and/or IL-2c at the indicated time-points. Livers were collected and analyzed five days after T_{N} transfer. (B) Representative confocal immunofluorescence micrographs of liver sections from the indicated mice 48 h after CLL treatment. KCs are depicted in red and sinusoids in grey. Scale bars represent 100 μm. (C-E) Representative flow cytometry plot (C) and absolute numbers (D) of Kuppfer cells from the indicated mice 48 h after CLL treatment. KCs are represented as CD31⁻, CD45⁺, TIM4⁺, F4/80+ cells. (E) Absolute numbers dendritic cells (MHC-II⁺, CD11c⁺) cells from the indicated mice 48 h after CLL treatment. (F-G) Total numbers (F) and numbers of IFN-γ-producing (G) T cells in the livers of indicated mice are shown. (H) Representative confocal immunofluorescence micrographs of liver sections from the indicated mice five days after T_{N} transfer. Cor93 T cells are depicted in green, Env28 T cells in red and sinusoids in grey. Scale bars represent 100 μm. (I) Schematic representation of the experimental setup. MUP-core mice were lethally irradiated and reconstituted with CD11c-DTR bone marrow (BM). Eight weeks after BM reconstitution, 1×10⁶ Cor93 T_{N} were transferred. Mice were treated with diphtheria toxin (DT) every other day starting from three days before T cell injection. Indicated mice received IL-2c at the indicated timepoints. Livers were collected and analyzed five days after T_{N} transfer. (J) Representative confocal immunofluorescence micrographs of liver sections from the indicated mice 48 h after CLL treatment. KCs are depicted in red and sinusoids in grey. Scale bars represent 100 μm. (K-M) Representative flow cytometry plot (K) and absolute numbers (L) of Kupffer cells from the indicated mice at the time of T cell transfer. (M) Absolute numbers dendritic cells (MHC-II+, CD11c+) cells from the indicated mice at the time of T cell transfer. (N-O) Total numbers (N) and numbers of IFN-γ-producing (O) T cells in the livers of the indicated mice. (P) Representative confocal immunofluorescence micrographs of liver sections from the indicated mice five days after T_{N} transfer. Cor93 T cells are depicted in green and sinusoids in grey. Scale bars represent 100 μm. Data are representative of at least 3 independent experiments. ** p value<0.01, *** p value<0.001

FIG. 31

KCs have a Functional IL-2-Sensing Machinery.

(A) Representative flow cytometry plot of CD25 (left panel), CD122 (middle panel), and CD132 (right panel) expression on CD45⁺ (blue) and F4/80⁺ (red) cell population in the liver. Isotype control is depicted in gray. (B) Mean Fluorescent Intensity (MFI) of CD25 (left), CD122 (middle), CD132 (right) expression on CD45⁺ (blue) and F4/80⁺ (red) cells in the liver. n=4. (C) Schematic representation of the experimental setup. Liver non parenchymal cells were isolated from C57BL/6 mice and incubated in vitro with PBS or IL-2c. After fifteen minutes pSTAT5 signal was analyzed on CD45⁺ F4/80⁺ TIM4⁺ cells (KCs) or CD31⁺ CD45⁻ cells (LSECs) by flow cytometry. (D) Fold change of pSTAT5 expression between IL-2 and PBS (control) condition on KCs (red dots) or LSECs (blue dots) incubated in vitro with the indicated IL-2c concentration. (E) Western blot analysis of STAT5/pSTAT5 in adherent KCs incubated in vitro with IL-2c or PBS. (F) Schematic representation of the experimental setup. C57BL/6 mice were treated in vivo with IL-2c or PBS as control. 48 hours after treatment, liver non parenchymal cells (NPCs) were isolated and RNAseq was performed on FACS-sorted KCs. (G) Representative KC gating strategy. (H) Clustering of top significant (EnrichR Combined Score >100, FDR<0.05) Gene Ontology Biological Processes and KEGG pathways of up-regulated processes. The thermal scale represents the Jaccard Similarity Coefficient between every gene set pair (blue representing a 0 Similarity Coefficient, red a 1 Similarity Coefficient). (I) Volcano plot of RNA-Seq results. The X-axis represent the Log 2 Fold-Change of Differentialy Expressed Genes upon IL-2c treatment, the Y-axis the −Log 10(FDR). Only DEGs with a FDR<0.05 were considered. Genes belonging to specific biological process were highlighted in different colours (FIG. 38A-D). (J) Radar plot of different biological process. Each dimension of the radar plot is represented as the mean of the TPM of selected genes (FIG. 38A-D), both in the control (blue) and in the IL-2c treated samples (red). Values range from 0 to 350 TPM. (K) Heatmap of expression values of upregulated genes linked to antigen presentation, upon IL-2c treatment, in isolated Kupffer cells. Values are in Z-score, calculated from scaling by row the Log 2(TPM) values. (L) MFI of H2-Kb, CD40 and CD80 markers on KCs in the indicated mice. (M) Schematic representation of the experimental setup. HBV-transgenic mice were treated in vivo with IL-2c or PBS. After 48 hours livers were harvested, NPCs were isolated and plated. KCs were seeded for 2 hours and co-cultured with in vitro-differentiated Cor93 effector T cells (Cor93 TE). After four hours, T cells were harvested and analyzed by flow cytometry. (N-O) Representative flow cytometry plot (N) and MFI (O) of IFNg producing TE cells in the indicated conditions. (P) Schematic representation of the experimental setup. C57BL/6 WT mice were treated in vivo with IL-2c or PBS. After 48 hours livers were harvested, NPCs were isolated, and KCs were immunomagnetically-purified. Purified KC were co-cultured with cell trace violet-labelled Cor93 naive T cells (CTV-Cor93 T_{N}). Indicated concentrations of HBeAg-containing mouse serum were added to the plate. After four days, T cells were harvested and analyzed by flow cytometry. (Q-R) Representative flow cytometry plot (Q) and percentages (R) of proliferating Cor93 T_{N} cells at the indicated conditions. (S) Schematic representation of the experimental setup. MUP-core mice were lethally irradiated and reconstituted with WT or TAP^{1-/-} bone marrow (BM). Eight weeks after BM reconstitution mice received two injection of clodronate liposomes (CLL) to remove residual radio-resistant KCs. Two weeks after the last dose of CLL, 5×10⁶ Cor93 T_{N} were transferred. Indicated mice received IL-2c at the indicated time-points. Livers were collected and analyzed five days after T_{N} transfer. (T-U) Total numbers (T) and numbers of IFN-γ-producing (U) T cells in the livers of the indicated mice.

Data are representative of at least 3 independent experiments. * p value<0.05, ** p value<0.01, *** p value<0.001

FIG. 32

KCs can be Sub-Divided into Two Populations with Respect to their IL-2 Sensing Capacity

(A) Representative flow cytometry plot of KC₁/KC₂ gating strategy. (B) Relative percentage of KC₁ and KC₂ among KC population in C57BL/6 WT mice. (C) Representative confocal immunofluorescence micrographs of liver sections from C57BL/6 WT mice. CD38⁺ cells are depicted in white, CD206⁺ cells in red, F4/80⁺ cells in green. Scale bars represent 10 μm. (D) GSEA relative to the HALLMARK_IL2_STAT5_SIGNALING Gene Set contained in MSigDB. Genes were pre-ranked based on the Log 2 Fold Change between KC2 in contrast to KC1. (E) Heatmap of expression of IL-2 receptor signaling pathway in KC1 and KC2, at basal level. Values in log 2TPM were scaled by row across samples (Z-score), (F-G) Representative flow cytometry plot and MFI (F) of CD25, CD122 and CD132 in KC₁, KC₂ and liver sinusoidal endothelial cells (LSEC) in C57BL/6 WT mice. (H-J) MFI of H2-Kb (H), CD40 (I) and CD80 (J) on KC₁ (blue) and KC₂ (red) from indicated mice. (K) Schematic representation of the experimental setup. HBV-Tg mice were injected with 10⁶ Cor93 T_{N} cells. Mice were treated with IL-2c or PBS as control one day after T cell transfer. Livers were collected and analyzed five days after T_{N} transfer. Representative flow cytometry plot (bottom panels) of KC₁ and KC₂ in the liver of indicated mice. (L) Ratio between KC₁ and KC₂ (left panel) and absolute numbers of KC₁ (middle panel) and KC₂ (right panel) in the liver of indicated mice. Data are representative of at least 3 independent experiments.

* p value<0.05, ** p value<0.01, *** p value<0.001

FIG. 33

KC₂ are Required for the Optimal Restoration of of Intrahepatically-Primed, Dysfunctional CD8+ T Cells by IL-2

(A) Schematic representation of the experimental setup. MUP-core mice were lethally irradiated and reconstituted with Cdh5-creERT2×Rosa26iDTRxCX3CR1-GFP bone marrow (BM). Ten weeks later mice received two injection of clodronate liposomes (CLL) to remove residual radio-resistant KCs. Nine weeks after BM reconstitution, mice were treated once with 5 mg of Tamoxifen by gavage. Mice were treated with diphteria toxin (DT) every other day starting from three days before T cell injection. Mice were injected with 1×10⁶ Cor93 T_{N}. Indicated mice received IL-2c one day after T cell transfer. Livers were collected and analyzed five days after T_{N} transfer. (B) Representative flow cytometry plot of KC₁/KC₂ population in the indicated mice at the time of T_{N} injection. (C-D) Total numbers (C) and numbers (D) of IFN-γ-producing Cor93 T cells in the livers of the indicated mice. (E) Levels of ALT in the serum of indicated mice at the indicated timepoint. (F) Representative confocal immunofluorescence micrographs of liver sections from the indicated mice five days after T_{N} transfer. Cor93 T cells are depicted in green and sinusoids in gray. Scale bars represent 100 μm.

Data are representative of at least 3 independent experiments. * p value<0.05, ** p value<0.01, *** p value<0.001

FIG. 34

Neutrophils and Monocytes are not are not Required for T Cell Reinvigoration Upon IL-2 Treatment

(A) Schematic representation of the experimental setup. 1×10⁶ Cor93 were transferred into HBV transgenic recipients. When indicated, mice were injected with anti-LY6G antibody one day before and one day after T cell injection. Indicated mice received IL-2c at the indicated timepoints. Livers were collected and analyzed five days after T_{N} transfer. (B-C) Numbers of neutrophils (B) and monocytes (C) in the blood in the indicated mice at the time of T cell injection. (D-E) Total numbers (D) and numbers of IFN-γ-producing (E) T cells in the livers of indicated mice are shown. (G) Schematic representation of the experimental setup. 1×10⁶ Cor93 were transferred into HBV transgenic recipients. When indicated, mice were injected with anti-Gr1 antibody every other day starting from 3 days before T cell injection. Indicated mice received IL-2c at the indicated timepoints. Livers were collected and analyzed five days after T_{N} transfer. (G-H) Numbers of neutrophils (G) and monocytes (H) in the blood in the indicated mice at the time of T cell injection. (I-J) Total numbers (I) and numbers of IFN-γ-producing (J) T cells in the livers of indicated mice are shown.

Data are representative of at least 3 independent experiments. **p value<0.01, *** p value<0.001

FIG. 35

pSTAT5 Expression on Tregs.

(A) Schematic representation of the experimental setup. Splenocytes were isolated from C57BL/6 mice and incubated in vitro with PBS or different concentrations of rIL-2. After fifteen minutes pSTAT5 signal was analyzed on live CD45⁺ CD4⁺ Foxp3⁺ cells by flow cytometry. (B) Fold change between rIL-2 and PBS conditions on live CD45⁺ CD4⁺ FOXP3⁺ cells incubated in vitro with the indicated IL-2c concentration.

Data are representative of at least 3 independent experiments. *** p value<0.001

FIG. 36

Gene Expression Profile of KCs after IL-2 Treatment

(A) PCA visualization of the samples used. (B) Heatmap of expression values of differentially expressed genes (FDR<0.05) upon IL-2c treatment in Kupffer cells. 1515 genes were up- and 2558 genes were down-regulated. Values in log 2TPM were scaled by row across samples (Z-score).

FIG. 37

Up-Regulated Processes in KCs after IL-2 Treatment

(A) Clustering of top significant (EnrichR Combined Score >100, FDR<0.05) Gene Ontology Biological Processes and KEGG pathways of up-regulated processes. The thermal scale represents the Jaccard Similarity Coefficient between every gene set pair (blue representing a 0 Similarity Coefficient, red a 1 Similarity Coefficient). (B) Clustering of top significant (EnrichR Combined Score >30, FDR<0.05) Gene Ontology Biological Processes and KEGG pathways of down-regulated processes. The thermal scale represents the Jaccard Similarity Coefficient between every gene set pair (blue representing a 0 Similarity Coefficient, red a 1 Similarity Coefficient).

FIG. 38

Cross-Presentation Processes are Upregulated in KCs after IL-2 Treatment

(A) Schematic representation and (B-E) expression heatmap of selected genes, belonging to biological processes implicated in antigen cross-presentation, upregulated in KCs after IL-2c treatment. Values in log 2TPM were scaled by row across samples (Z-score). (F) Network of top significant (EnrichR Combined Score >100, FDR<0.05) Gene Ontology Biological Processes and KEGG pathways of up-regulated processes, made using Cytoscape software. Red dots indicate enriched terms, green dots indicate the relative genes found enriched.

FIG. 39

KC Enrichment after Immunomagnetic Sorting

(A) Representative flow cytometry plot of KC fraction (CD45⁺ F4/80⁺) in the liver non parenchymal cell population before (left panel) and after (right panel) negative immunomagnetic sorting. (B) Representative flow cytometry plot of DC fraction (CD11c⁺ MHC-II⁺) in the liver non parenchymal cell population before (left panel) and after (right panel) negative immunomagnetic sorting. Data are representative of at least 3 independent experiments.

FIG. 40

TAP1^{-/-} Mice Show Similar Percentages of KCs in the Liver

(A-B) Representative histogram (A) and MFI (B) of H2-Kb expression on KCs (CD45⁺ F4/80⁺) isolated from C57BL/6 (blue line) or TAP1^{-/-} (red line) mice (B) Percentage of KCs upon CD45⁺ liver non parenchymal cells in indicated mice.

Data are representative of at least 3 independent experiments. *** p value<0.001

FIG. 41

Gene Expression Profile of KC₁ and KC₂ after IL-2 Treatment

(A) Heatmap of expression values of differentially expressed genes (FDR<0.05) between KC1 and KC2 at basal level. 3424 genes were up- and 4153 genes were down-regulated in KC2 in contrast to KC1. Values in log 2TPM were scaled by row across samples (Z-score).

FIG. 42

IL-2 Alone or Liver Inflammation have No Impact on KC₁/KC₂ Ratio

(A) Schematic representation of the experimental setup. HBV Tg mice were treated with PBS or IL-2c and livers were collected and analyzed 4 days after treatment. (B) Numbers of KCs/gr of liver in the indicated mice. (C) Representative flow cytometry plot of KC₁ (CD206⁻ ESAM⁻) and KC₂ (CD206⁺ ESAM⁺) in the indicated mice. (D) Numbers of KC₁ and KC₂ per gr of liver in the indicated mice. (E) Schematic representation of the experimental setup. MUP-core mice were treated with IL-2c or PBS one day before Cor93 T effector cell transfer. Livers were collected and analyzed 2 days after T cell transfer. (F) Levels of ALT in the serum of indicated mice at the indicated timepoints. (G-I) Numbers of liver non parenchymal cells (G), Cor93 T cells (H) and KCs (I) in the liver of indicated mice in the indicated conditions. (J) Representative flow cytometry plot of KC₁ (CD206⁻ ESAM⁻) and KC2 (CD206⁺ ESAM⁺) in the indicated mice. (K-L) Percentages (K) and numbers (L) of KC₁ and KC₂ in the liver of indicated mice in the indicated conditions.

Data are representative of at least 3 independent experiments. *p value<0.05, *** p value<0.001

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Hepatitis

The invention relates to agents for use in the treatment and prevention of viral infections, in particular viral liver infections, such as hepatitis infections.

Hepatitis infections, such as hepatitis B virus (HBV) infection, remain a major public health issue worldwide. For example, it has been estimated that about 248 million individuals were positive for hepatitis B surface antigen, a marker of chronic HBV infection, globally in 2010.

HBV is a non-cytopathic virus that replicates exclusively in hepatocytes without inducing innate immune activation.

Chronic HBV infection is typically acquired at birth or in early childhood, and is particularly prevalent in Asian and African countries where HBV is endemic. The risk of developing chronic infection after exposure drops from ca. 90% in neonates to 1-5% in healthy adults. However, 25% of people who acquire HBV as children will develop primary liver cancer or cirrhosis as adults.

Chronic infections acquired perinatally or in early childhood go through several prolonged and progressive disease phases, including an initial “immune tolerant” phase (characterised by high viremia, normal ALT values and no liver inflammation) that is often followed by an “immune active” phase (in which viremia is lower, ALT values are higher and liver inflammation is present) (Kennedy, P. et al. (2017) Viruses 9: 96; and EASL (2017) Journal of Hepatology 67: 370-398). HBV-specific CD8⁺ T cells in young immune tolerant chronic HBV patients are considered akin to Ag-specific exhausted T cells that characterise the immune active phase (Fisicaro, P. et al. (2017) Nature Medicine 23: 327-336), as well as to other infection- or cancer-related conditions of immune dysfunction.

HBV and HCV infections can both give rise to hepatocellular carcinomas.

Interleukin

Interleukins (ILs) are a group of cytokines, the majority of which are made by helper CD4 T cells, as well as monocytes, macrophages and endothelial cells. They function in promoting the development and differentiation of T and B lymphocytes, and hematopoietic cells.

The vectors of the invention may comprise a nucleotide sequence encoding an interleukin which binds to IL-2 receptor (IL-2R). In some embodiments, the interleukin is selected from the group consisting of IL-2, IL-7 or IL-15, preferably the interleukin is IL-2.

Interleukin-2 (IL-2)

Interleukin-2 (IL-2) plays a role in the regulation of the activities of white blood cells that are responsible for immunity. IL-2 is part of the natural response to microbial infection, and is involved in the discrimination between “self” and “non-self”. IL-2 mediates its effects by binding to IL-2 receptors, which are expressed by lymphocytes. Sources of IL-2 include activated CD4+ T cells, activated CD8+ T cells, NK cells, dendritic cells and macrophages.

In preferred embodiments, the IL-2 is human IL-2.

An example IL-2 sequence is:

(SEQ ID NO: 1)
MYRMQLLSCIALSLALVTNSAPTSSSTKKTQLQLEHLLLDLQMILNGINNY
KNPKLTRMLTFKFYMPKKATELKHLQCLEEELKPLEEVLNLAQSKNFHLRP
RDLISNINVIVLELKGSETTFMCEYADETATIVEFLNRWITFCQSIISTLT

An example nucleotide sequence encoding IL-2 is:

(SEQ ID NO: 2)
ATGTACAGGATGCAACTCCTGTCTTGCATTGCACTAAGTCTTGCACTTGTC
ACAAACAGTGCACCTACTTCAAGTTCTACAAAGAAAACACAGCTACAACTG
GAGCATTTACTGCTGGATTTACAGATGATTTTGAATGGAATTAATAATTAC
AAGAATCCCAAACTCACCAGGATGCTCACATTTAAGTTTTACATGCCCAAG
AAGGCCACAGAACTGAAACATCTTCAGTGTCTAGAAGAAGAACTCAAACCT
CTGGAGGAAGTGCTAAATTTAGCTCAAAGCAAAAACTTTCACTTAAGACCC
AGGGACTTAATCAGCAATATCAACGTAATAGTTCTGGAACTAAAGGGATCT
GAAACAACATTCATGTGTGAATATGCTGATGAGACAGCAACCATTGTAGAA
TTTCTGAACAGATGGATTACCTTTTGTCAAAGCATCATCTCAACACTGACT
TGA

In some embodiments, the IL-2 is encoded by a nucleotide sequence that has at least 70%, 80%, 90%, 95%, 96%, 97%, 98% 99% or 100% identity to SEQ ID NO: 2, preferably wherein the protein encoded by the nucleotide sequence substantially retains the natural function of the protein represented by SEQ ID NO: 1.

In some embodiments, the IL-2 is encoded by a nucleotide sequence that encodes an amino acid sequence that has at least 70%, 80%, 90%, 95%, 96%, 97%, 98% 99% or 100% identity to SEQ ID NO: 1, preferably wherein the amino acid sequence substantially retains the natural function of the protein represented by SEQ ID NO: 1.

In some embodiments, the IL-2 comprises or consists of an amino acid sequence that has at least 70%, 80%, 90%, 95%, 96%, 97%, 98% 99% or 100% identity to SEQ ID NO: 1, preferably wherein the amino acid sequence substantially retains the natural function of the protein represented by SEQ ID NO: 1.

Interleukin-7 (IL-7)

Interleukin-7 (IL-7) is a hematopoietic growth factor that may be secreted by stromal cells in the bone marrow and thymus. IL-7 may also be produced by keratinocytes, dendritic cells, hepatocytes, neurons and epithelial cells, but is typically not produced by normal lymphocytes.

In preferred embodiments, the IL-7 is human IL-7.

An example IL-7 sequence is:

(SEQ ID NO: 3)
MFHVSFRYIEGLPPLILVLLPVASSDCDIEGKDGKQYESVLMVSIDQLLDS
MKEIGSNCLNNEFNFFKRHICDANKEGMFLFRAARKLRQFLKMNSTGDFDL
HLLKVSEGTTILLNCTGQVKGRKPAALGEAQPTKSLEENKSLKEQKKLNDL
CFLKRLLQEIKTCWNKILMGTKEH

An example nucleotide sequence encoding IL-7 is:

(SEQ ID NO: 4)
ATGTTCCATGTTTCTTTTAGGTATATCTTTGGACTTCCTCCCCTGATCCTT
GTTCTGTTGCCAGTAGCATCATCTGATTGTGATATTGAAGGTAAAGATGGC
AAACAATATGAGAGTGTTCTAATGGTCAGCATCGATCAATTATTGGACAGC
ATGAAAGAAATTGGTAGCAATTGCCTGAATAATGAATTTAACTTTTTTAAA
AGACATATCTGTGATGCTAATAAGGAAGGTATGTTTTTATTCCGTGCTGCT
CGCAAGTTGAGGCAATTTCTTAAAATGAATAGCACTGGTGATTTTGATCTC
CACTTATTAAAAGTTTCAGAAGGCACAACAATACTGTTGAACTGCACTGGC
CAGGTTAAAGGAAGAAAACCAGCTGCCCTGGGTGAAGCCCAACCAACAAAG
AGTTTGGAAGAAAATAAATCTTTAAAGGAACAGAAAAAACTGAATGACTTG
TGTTTCCTAAAGAGACTATTACAAGAGATAAAAACTTGTTGGAATAAAATT
TTGATGGGCACTAAAGAACACTGA

In some embodiments, the IL-7 is encoded by a nucleotide sequence that has at least 70%, 80%, 90%, 95%, 96%, 97%, 98% 99% or 100% identity to SEQ ID NO: 4, preferably wherein the protein encoded by the nucleotide sequence substantially retains the natural function of the protein represented by SEQ ID NO: 3.

In some embodiments, the IL-7 is encoded by a nucleotide sequence that encodes an amino acid sequence that has at least 70%, 80%, 90%, 95%, 96%, 97%, 98% 99% or 100% identity to SEQ ID NO: 3, preferably wherein the amino acid sequence substantially retains the natural function of the protein represented by SEQ ID NO: 3.

In some embodiments, the IL-7 comprises or consists of an amino acid sequence that has at least 70%, 80%, 90%, 95%, 96%, 97%, 98% 99% or 100% identity to SEQ ID NO: 3, preferably wherein the amino acid sequence substantially retains the natural function of the protein represented by SEQ ID NO: 3.

Interleukin-15 (IL-15)

Interleukin-15 (IL-15) has structural similarity to IL-2. IL-15 is secreted by mononuclear phagocytes following viral infection. It induces proliferation of natural killer cells.

In preferred embodiments, the IL-15 is human IL-15.

An example IL-15 sequence is:

(SEQ ID NO: 5)
MRISKPHLRSISIQCYLCLLLNSHFLTEAGIHVFILGCFSAGLPKTEANWV
NVISDLKKIEDLIQSMHIDATLYTESDVHPSCKVTAMKCFLLELQVISLES
GDASIHDTVENLIILANNSLSSNGNVTESGCKECEELEEKNIKEFLQSFVH
IVQMFINTS

An example nucleotide sequence encoding IL-15 is:

(SEQ ID NO: 6)
ATGAGAATTTCGAAACCACATTTGAGAAGTATTTCCATCCAGTGCTACTTG
TGTTTACTTCTAAACAGTCATTTTCTAACTGAAGCTGGCATTCATGTCTTC
ATTTTGGGCTGTTTCAGTGCAGGGCTTCCTAAAACAGAAGCCAACTGGGTG
AATGTAATAAGTGATTTGAAAAAAATTGAAGATCTTATTCAATCTATGCAT
ATTGATGCTACTTTATATACGGAAAGTGATGTTCACCCCAGTTGCAAAGTA
ACAGCAATGAAGTGCTTTCTCTTGGAGTTACAAGTTATTTCACTTGAGTCC
GGAGATGCAAGTATTCATGATACAGTAGAAAATCTGATCATCCTAGCAAAC
AACAGTTTGTCTTCTAATGGGAATGTAACAGAATCTGGATGCAAAGAATGT
GAGGAACTGGAGGAAAAAAATATTAAAGAATTTTTGCAGAGTTTTGTACAT
ATTGTCCAAATGTTCATCAACACTTCTTGA

In some embodiments, the IL-15 is encoded by a nucleotide sequence that has at least 70%, 80%, 90%, 95%, 96%, 97%, 98% 99% or 100% identity to SEQ ID NO: 6, preferably wherein the protein encoded by the nucleotide sequence substantially retains the natural function of the protein represented by SEQ ID NO: 5.

In some embodiments, the IL-15 is encoded by a nucleotide sequence that encodes an amino acid sequence that has at least 70%, 80%, 90%, 95%, 96%, 97%, 98% 99% or 100% identity to SEQ ID NO: 5, preferably wherein the amino acid sequence substantially retains the natural function of the protein represented by SEQ ID NO: 5.

In some embodiments, the IL-15 comprises or consists of an amino acid sequence that has at least 70%, 80%, 90%, 95%, 96%, 97%, 98% 99% or 100% identity to SEQ ID NO: 5, preferably wherein the amino acid sequence substantially retains the natural function of the protein represented by SEQ ID NO: 5.

Expression Control Sequences

The vector of the invention may include elements allowing for the expression of the nucleotide sequence encoding interleukin. These may be referred to as expression control sequences. Thus, the vector may comprise one or more expression control sequences (e.g. comprising a promoter sequence) operably linked to the nucleotide sequence encoding the interleukin.

By “operably linked”, it is to be understood that the individual components are linked together in a manner which enables them to carry out their function substantially unhindered (e.g. a promoter may be operably linked to a nucleotide of interest to promote expression of the nucleotide of interest in a cell).

Promoters and Enhancers

Any suitable promoter and/or enhancer may be used, the selection of which may be readily made by the skilled person. The promoter sequence may be constitutively active (i.e. operational in any host cell background), or alternatively may be active only in a specific host cell environment, thus allowing for targeted expression of the nucleotide of interest (e.g. the interleukin) in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

Preferably, the expression control sequences enable liver-specific expression of the interleukin, for example confined only to liver cells, such as hepatocytes. Examples of liver-specific promoters include the hepatocyte-specific promoters, liver sinusoidal endothelial cell-specific promoters and Kupffer cell-specific promoters disclosed herein (e.g. selected from the group consisting of an ET promoter, albumin promoter, transthyretin promoter, alpha1-antitrypsin promoter, apoE/alpha1-antitrypsin promoter, vascular endothelial cadherin (VEC) promoter, intercellular adhesion molecule 2 (ICAM2) promoter, foetal liver kinase 1 (Flk1) promoter, Tie2 promoter and CD11b promoter).

In some embodiments, the vector comprises a hepatocyte-specific promoter and/or enhancer operably linked to the nucleotide sequence encoding the interleukin.

In some embodiments, the hepatocyte-specific promoter is selected from the group consisting of an ET promoter, albumin promoter, transthyretin promoter, alpha1-antitrypsin promoter and apoE/alpha1-antitrypsin promoter.

The hepatocyte-specific Enhanced Transthyretin (ET) promoter is described in Vigna, E. et al. (2005) Mol. Ther. 11: 763-775, and is composed of synthetic hepatocyte-specific enhancers and transthyretin promoter.

In preferred embodiments, the promoter is an ET promoter.

An example ET promoter sequence is:

(SEQ ID NO: 8)
CGCGAGTTAATAATTACCAGCGCGGGCCAAATAAATAATCCGCGAGGGGCA
GGTGACGTTTGCCCAGCGCGCGCTGGTAATTATTAACCTCGCGAATATTGA
TTCGAGGCCGCGATTGCCGCAATCGCGAGGGGCAGGTGACCTTTGCCCAGC
GCGCGTTCGCCCCGCCCCGGACGGTATCGATAAGCTTAGGAGCTTGGGCTG
CAGGTCGAGGGCACTGGGAGGATGTTGAGTAAGATGGAAAACTACTGATGA
CCCTTGCAGAGACAGAGTATTAGGACATGTTTGAACAGGGGCCGGGCGATC
AGCAGGTAGCTCTAGAGGATCCCCGTCTGTCTGCACATTTCGTAGAGCGAG
TGTTCCGATACTCTAATCTCCCTAGGCAAGGTTCATATTTGTGTAGGTTAC
TTATTCTCCTTTTGTTGACTAAGTCAATAATCAGAATCAGCAGGTTTGGAG
TCAGCTTGGCAGGGATCAGCAGCCTGGGTTGGAAGGAGGGGGTATAAAAGC
CCCTTCACCAGGAGAAGCCGTCACACAGATCCACAAGCTCCTG

In some embodiments, the vector comprises a promoter with a nucleotide sequence that has at least 75%, 80%, 85% 90%, 95%, 96%, 97%, 98% or 99% identity to SEQ ID NO: 8 operably linked to the nucleotide sequence encoding the interleukin. Preferably, wherein the promoter substantially retains the functional activity of the promoter represented by SEQ ID NO: 8.

In other embodiments, the vector comprises a promoter with the nucleotide sequence of SEQ ID NO: 8 operably linked to the nucleotide sequence encoding the interleukin.

The albumin promoter is described in Follenzi, A. et al (2004) Blood 103: 3700-3709.

An example albumin promoter sequence is:

(SEQ ID NO: 9)
GGCATGCTTCCATGCCAAGGCCCACACTGAAATGCTCAAATGGGAGACAAA
GAGATTAAGCTCTTATGTAAAATTTGCTGTTTTACATAACTTTAATGAATG
GACAAAGTCTTGTGOATGGGGGTGGGGGTGGGGTTAGAGGGGAACAGCTCC
AGATGGCAAACATACGCAAGGGATTTAGTCAAACAACTTTTTGGCAAAGAT
GGTATGATTTTGTAATGGGGTAGGAACCAATGAAATGCGAGGTAAGTATGG
TTAATGATCTACAGTTATTGGTTAAAGAAGTATATTAGAGCGAGTCTTTCT
GCACACAGATCACCTTTCCTATCAACCCC

In some embodiments, the vector comprises a promoter with a nucleotide sequence that has at least 75%, 80%, 85% 90%, 95%, 96%, 97%, 98% or 99% identity to SEQ ID NO: 9 operably linked to the nucleotide sequence encoding the interleukin. Preferably, wherein the promoter substantially retains the functional activity of the promoter represented by SEQ ID NO: 9.

In other embodiments, the vector comprises a promoter with the nucleotide sequence of SEQ ID NO: 9 operably linked to the nucleotide sequence encoding the interleukin.

An alpha1-antitrypsin (AAT) promoter is described in Hafenrichter, D. G. et al. (1994) Blood 84: 3394-3404 and WO2016146757, and an apoE/alpha1-antitrypsin promoter is described in Miao, C. H. et al. (2000) Mol Ther 1: 522-532 and WO2001098482.

Other suitable promoters, which are not liver specific, include the PGK promoter.

MicroRNA (miRNA) Target Sequences

The vector of the invention may comprise elements which prevent or reduce the expression of the encoded transgene, for example in certain tissues. Such elements could be recognition sequences which bind or interact with modulators. The modulators could be endogenous modulators present in a cell. Alternatively, the modulators could be exogenous molecules which are introduced into the cell. Preferably, the modulators are microRNAs.

MicroRNA genes are scattered across all human chromosomes, except for the Y chromosome. They can be either located in non-coding regions of the genome or within introns of protein-coding genes. Around 50% of miRNAs appear in clusters which are transcribed as polycistronic primary transcripts. Similar to protein-coding genes, miRNAs are usually transcribed from polymerase-II promoters, generating a so-called primary miRNA transcript (pri-miRNA). This pri-miRNA is then processed through a series of endonucleolytic cleavage steps, performed by two enzymes belonging to the RNAse Type III family, Drosha and Dicer. From the pri-miRNA, a stem loop of about 60 nucleotides in length, called miRNA precursor (pre-miRNA), is excised by a specific nuclear complex, composed of Drosha and DiGeorge syndrome critical region gene (DGCR8), which crops both strands near the base of the primary stem loop and leaves a 5′ phosphate and a 2 bp long, 3′ overhang. The pre-miRNA is then actively transported from the nucleus to the cytoplasm by RAN-GTP and Exportin. Then, Dicer performs a double strand cut at the end of the stem loop not defined by the Drosha cut, generating a 19-24 bp duplex, which is composed of the mature miRNA and the opposite strand of the duplex, called miRNA*. In agreement with the thermodynamic asymmetry rule, only one strand of the duplex is selectively loaded into the RNA-induced silencing complex (RISC), and accumulates as the mature microRNA. This strand is usually the one whose 5′ end is less tightly paired to its complement, as was demonstrated by single-nucleotide mismatches introduced into the 5′ end of each strand of siRNA duplexes. However, there are some miRNAs that support accumulation of both duplex strands to similar extent.

MicroRNAs trigger RNAi, very much like small interfering RNAs (siRNA) which are extensively used for experimental gene knockdown. The main difference between miRNA and siRNA is their biogenesis. Once loaded into RISC, the guide strand of the small RNA molecule interacts with mRNA target sequences preferentially found in the 3′ untranslated region (3′UTR) of protein-coding genes. It has been shown that nucleotides 2-8 counted from the 5′ end of the miRNA, the so-called seed sequence, are essential for triggering RNAi. If the whole guide strand sequence is perfectly complementary to the mRNA target, as is usually the case for siRNAs and plant miRNAs, the mRNA is endonucleolytically cleaved by involvement of the Argonaute (Ago) protein, also called “slicer” of the small RNA duplex into the RNA-induced silencing complex (RISC). DGRC (DiGeorge syndrome critical region gene 8) and TRBP (TAR (HIV) RNA binding protein 2) are double-stranded RNA-binding proteins that facilitate mature miRNA biogenesis by Drosha and Dicer RNase III enzymes, respectively. The guide strand of the miRNA duplex gets incorporated into the effector complex RISC, which recognises specific targets through imperfect base-pairing and induces post-transcriptional gene silencing. Several mechanisms have been proposed for this mode of regulation: miRNAs can induce the repression of translation initiation, mark target mRNAs for degradation by deadenylation, or sequester targets into the cytoplasmic P-body.

On the other hand, if only the seed is perfectly complementary to the target mRNA but the remaining bases show incomplete pairing, RNAi acts through multiple mechanisms leading to translational repression. Eukaryotic mRNA degradation mainly occurs through the shortening of the polyA tail at the 3′ end of the mRNA, and de-capping at the 5′ end, followed by 5′-3′ exonuclease digestion and accumulation of the miRNA in discrete cytoplasmic areas, the so called P-bodies, enriched in components of the mRNA decay pathway.

According to the present invention, expression of the interleukin, such as IL-2, may be regulated by endogenous miRNAs using corresponding miRNA target sequences. Using this method, a miRNA endogenously expressed in a cell prevents or reduces transgene expression in that cell by binding to its corresponding miRNA target sequence positioned in the vector or polynucleotide (Brown, B. D. et al. (2007) Nat Biotechnol 25: 1457-1467).

miRNA target sequences that are useful in the present invention include miRNA target sequences which are expressed in haematopoietic cells.

Preferably, the target sequence is the target of an miRNA selected from the group consisting of miR-142, miR-155 and miR-223.

In some embodiments, the nucleotide sequence encoding the interleukin is operably linked to one or more miR-142, miR-155 and/or miR-223 target sequences. In preferred embodiments, the nucleotide sequence is operably linked to one or more miR-142 target sequences.

An example miR-142 target sequence is:

(SEQ ID NO: 10)
TCCATAAAGTAGGAAACACTACA

An example miR-155 target sequence is:

(SEQ ID NO: 11)
CCCCTATCACGATTAGCATTAA

An example miR-223 target sequence is:

(SEQ ID NO: 12)
GGGGTATTTGACAAACTGACA

More than one copy of an miRNA target sequence included in the vector may increase the effectiveness of the system. Also it is envisaged that different miRNA target sequences could be included. For example, vectors which express more than one transgene may have the transgene under control of more than one miRNA target sequence, which may or may not be different. The miRNA target sequences may be in tandem, but other arrangements are envisaged. Preferably, the vector comprises 1, 2, 3, 4, 5, 6, 7 or 8 copies of the same or different miRNA target sequence. In preferred embodiments, the vector comprises 4 miR-142 target sequences.

In some embodiments, the target sequence is fully or partially complementary to the miRNA. The term “fully complementary”, as used herein, may mean that the target sequence has a nucleic acid sequence which is 100% complementary to the sequence of the miRNA which recognises it. The term “partially complementary”, as used herein, may mean that the target sequence is only in part complementary to the sequence of the miRNA which recognises it, whereby the partially complementary sequence is still recognised by the miRNA. In other words, a partially complementary target sequence in the context of the present invention is effective in recognising the corresponding miRNA and effecting prevention or reduction of transgene expression in cells expressing that miRNA.

Copies of miRNA target sequences may be separated by a spacer sequence. The spacer sequence may comprise, for example, at least one, at least two, at least three, at least four or at least five nucleotide bases.

Further Regulatory Elements

The vector of the invention may also comprise one or more additional regulatory sequences with may act pre- or post-transcriptionally. The regulatory sequence may be part of the native transgene locus or may be a heterologous regulatory sequence. The vector of the invention may comprise portions of the 5′-UTR or 3′-UTR from the native transgene transcript.

Regulatory sequences are any sequences which facilitate expression of the transgene, i.e. act to increase expression of a transcript, improve nuclear export of mRNA or enhance its stability. Such regulatory sequences include for example post-transcriptional regulatory elements and polyadenylation sites.

A preferred post-transcriptional regulatory element for use in a vector of the invention is the woodchuck hepatitis post-transcriptional regulatory element (WPRE) or a variant thereof.

An example WPRE sequence is:

(SEQ ID NO: 7)
ATCAACCTCTGGATTACAAAATTTGTGAAAGATTGACTGGTATTCTTAACT
ATGTTGCTCCTTTTACGCTATGTGGATACGCTGCTTTAATGCCTTTGTATC
ATGCTATTGCTTCCCGTATGGCTTTCATTTTCTCCTCCTTGTATAAATCCT
GGTTGCTGTCTCTTTATGAGGAGTTGTGGCCCGTTGTCAGGCAACGTGGCG
TGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCA
CGGCGGAACTCATCGCCGCCTGCCTTGCCCGCTGCTGGACAGGGGCTCGGC
TGTTGGGCACTGACAATTCCGTGGTGTTGTCGGGGAAATCATCGTCCTTTC
CTTGGCTGCTCGCCTGTGTTGCCACCTGGATTCTGCGCGGGACGTCCTTCT
GCTACGTCCCTTCGGCCCTCAATCCAGCGGACCTTCCTTCCCGCGGCCTGC
TGCCGGCTCTGCGGCCTCTTCCGCGTCTTCGCCTTCGCCCTCAGACGAGTC
GGATCTCCCTTTGGGCCGCCTCCCCGC

The invention encompasses the use of any variant sequence of the WPRE which increases expression of the transgene compared to a vector without a WPRE.

Vectors

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid and/or facilitating the expression of the protein encoded by a segment of nucleic acid.

Vectors comprising polynucleotides used in the invention may be introduced into cells using a variety of techniques known in the art, such as transfection, transduction and transformation.

Transfection may refer to a general process of incorporating a nucleic acid into a cell and includes a process using a non-viral vector to deliver a polynucleotide to a cell. Transduction may refer to a process of incorporating a nucleic acid into a cell using a viral vector.

The vector of the invention may be adapted for liver-specific expression of the nucleotide sequence encoding the interleukin.

The term “adapted for liver-specific expression”, as used herein, may refer to preferential expression of the nucleotide sequence in liver tissue, preferably hepatocytes, in comparison to other tissue of a subject. Preferably, no or substantially no expression of the nucleotide sequence occurs in non-liver tissue. The skilled person is readily able to determine expression profiles of a nucleotide sequence using methods known in the art, for example analysing protein and/or mRNA levels in specific cell types obtained from a subject using techniques such as Western blot.

A vector adapted for liver-specific expression may comprise suitable liver-specific expression control sequences, for example as disclosed herein, and/or may be in a form that preferentially transfects, transduces or transforms liver cells, such as hepatocytes.

Preferably, the vector is a viral vector. The vectors of the invention are preferably lentiviral vectors, although it is contemplated that other viral vectors may be used.

Preferably, the viral vector for use according to the invention is in the form of a viral vector particle.

In some embodiments, the vector is an RNA (e.g. mRNA) vector.

Transduction of cells with RNA vectors can be achieved, for example, using liposomes or lipid nanoparticles. In some embodiments, the RNA vector is in the form of a liposome or lipid nanoparticle.

Liposomes may naturally preferentially target hepatocytes. Thus, a vector in the form of a liposome may be adapted for liver-specific expression in the absence of liver-specific expression control sequences. However, it is envisaged that a vector in the form of a liposome may suitably comprise one or more liver-specific expression control sequences, preferably one or more miR-142, miR-155 and/or miR-223 target sequences, preferably further a hepatocyte-specific promoter and/or enhancer.

Lipid nanoparticles may be modified to preferentially target hepatocytes, for example the lipid nanoparticles may comprise a hepatocyte-specific ligand, such as N-acetyl-D-galactosamine (GaINAc).

Retro Viral and Lentiviral Vectors

A retroviral vector may be derived from or may be derivable from any suitable retrovirus. A large number of different retroviruses have been identified. Examples include murine leukaemia virus (MLV), human T cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, P et al. (1992) EMBO J. 11: 3053-8; Lewis, P. F. et al. (1994) J. Virol. 68: 510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans).

Examples of non-primate lentiviruses may be any member of the family of lentiviridae which does not naturally infect a primate.

As examples of lentivirus-based vectors, HIV-1- and HIV-2-based vectors are described below.

In preferred embodiments, the vector is an HIV vector, such as a HIV-1 or HIV-2 vector, preferably a HIV-1 vector.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE.

Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

In some embodiments, the vector is an integration-defective lentiviral vector (IDLV).

Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272: 263-7; Naldini, L. et al. (1996) Proc. Natl. Acad. Sci. USA 93: 11382-8; Leavitt, A. D. et al. (1996) J. Virol. 70: 721-8) or by modifying or deleting essential att sequences from the vector LTR (Nightingale, S. J. et al. (2006) Mol. Ther. 13: 1121-32), or by a combination of the above.

Adeno-Associated Viral (AAV) Vectors

Adeno-associated virus (AAV) has a high frequency of integration and it can infect non-dividing cells. This makes it useful for delivery of genes into mammalian cells in tissue culture.

AAV has a broad host range for infectivity. Details concerning the generation and use of AAV vectors are described in U.S. Pat. Nos. 5,139,941 and 4,797,368.

Recombinant AAV vectors have been used successfully for in vitro and in vivo transduction of marker genes and genes involved in human diseases.

Adenoviral Vectors

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms.

Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

Protein Transduction

As an alternative to the delivery of polynucleotides, such as using vectors, the interleukins of the invention may be delivered to cells as proteins, such as by protein transduction.

Protein transduction may be via vector delivery (Cai, Y. et al. (2014) Elife 3: e01911; Maetzig, T. et al. (2012) Curr. Gene Ther. 12: 389-409). Vector delivery involves the engineering of viral particles (e.g. lentiviral particles) to comprise the proteins to be delivered to a cell. Accordingly, when the engineered viral particles enter a cell as part of their natural life cycle, the proteins comprised in the particles are carried into the cell.

Protein transduction may be via protein delivery (Gaj, T. et al. (2012) Nat. Methods 9: 805-7). Protein delivery may be achieved, for example, by utilising a vehicle (e.g. a nanoparticle or liposome).

In some embodiments, the interleukin is in complex with a an anti-interleukin antibody, preferably a non-neutralising antibody. In some embodiments, the IL-2 is in complex with an anti-IL-2 antibody. In some embodiments, the IL-7 is in complex with an anti-IL-7 antibody. In some embodiments, the IL-15 is in complex with an anti-IL-15 antibody.

In some embodiments, the interleukin is comprised in a nanoparticle or liposome.

In one aspect, the invention provides an interleukin which binds to IL 2 receptor (IL-2R) which is adapted to be targeted to the liver. In another aspect, the invention provides interleukin-2 (IL-2), interleukin-7 (IL-7) and/or interleukin-15 (IL-15), wherein the IL-2, IL-7 and/or IL-15 is adapted to be targeted to the liver.

The term “adapted to be targeted to the liver”, as used herein, may refer to preferential delivery of the interleukin to liver tissue, preferably hepatocytes, in comparison to other tissue of a subject. Preferably, no or substantially no interleukin targeted in said way is delivered to or accumulated in non-liver tissue. The skilled person is readily able to determine delivery profiles using methods known in the art, for example analysing protein levels in specific cell types obtained from a subject using techniques such as Western blot.

Targeting to the liver may be achieved for example using nanoparticles or liposomes that are adapted to be targeted to the liver.

The interleukin, nanoparticle and/or liposome may be, for example, adapted to be targeted to a specific liver cell type. In some embodiments, the targeting is to hepatocytes. In some embodiments, the targeting is to liver sinusoidal endothelial cells. In some embodiments, the targeting is to Kupffer cells.

In some embodiments, the nanoparticle or liposome comprises a liver-specific ligand. The liver-specific ligand may be, for example a hepatocyte-, liver sinusoidal endothelial cell- or Kupffer cell-specific ligand.

Examples of suitable ligands and their target liver cell type, and further means of targeting nanoparticles or liposomes (e.g. passive targeting means) are described in the table below.

| Liver cell type | Cellular target | Targeting ligand or means | Reference |
|---|---|---|---|
| Hepatic stellate | Mannose-6- | Mannose-6-phosphate | M. Moreno, T. Gonzalo, R. J. |
| cells | phosphate | | Kok, P. Sancho-Bru, M. van |
| | receptor | | Beuge, J. Swart, et al., |
| | | | Hepatology 51 (2010) 942; |
| | | | N. Yang, Z. Ye, F. Li, R. I. |
| | | | Mahato, Bioconjug. Chem. |
| | | | 20 (2009) 213; W. I. Hagens, |
| | | | A. Mattos, R. Greupink, A. de |
| | | | Jager-Krikken, C. Reker- |
| | | | Smit, A. van Loenen- |
| | | | Weemaes, et al., Pharm. |
| | | | Res. 24 (2007) 566; J. E. |
| | | | Adrian, K. Poelstra, G. L. |
| | | | Scherphof, G. Molema, D. K. |
| | | | Meijer, C. Reker-Smit, et al., |
| | | | J. Hepatol. 44 (2006) 560; |
| | | | J. E. Adrian, J. A. Kamps, G. L. |
| | | | Scherphof, D. K. Meijer, A. M. |
| | | | van Loenen-Weemaes, C. |
| | | | Reker-Smit, et al., Biochim. |
| | | | Biophys. Acta 1768 (2007) |
| | | | 1430; J. E. Adrian, K. |
| | | | Poelstra, G. L. Scherphof, |
| | | | D. K. Meijer, A. M. van |
| | | | Loenen-Weemaes, C. Reker- |
| | | | Smit, et al., J. Pharmacol. |
| | | | Exp. Ther. 321 (2007) 536; |
| | | | Z. Ye, K. Cheng, R. V. |
| | | | Guntaka, R. I. Mahato, |
| | | | Biochemistry (Mose). 44 |
| | | | (2005) 4466. |
| | Retinol binding | Vitamin A | Y. Sato, K. Murase, J. Kato, |
| | protein receptor | | M. Kobune, T. Sato, Y. |
| | | | Kawano, et al., Nat. |
| | | | Biotechnol. 26 (2008) 431. |
| | Type VI | Cyclic RGD | L. Beljaars, G. Molema, D. |
| | collagen | | Schuppan, A. Geerts, P. J. |
| | receptor | | De Bleser, B. Weert, et al., J. |
| | | | Biol. Chem. 275 (2000) |
| | | | 12743; S. L. Du, H. Pan, W. Y. |
| | | | Lu, J. Wang, J. Wu, J. Y. |
| | | | Wang, J. Pharmacol. Exp. |
| | | | Ther. 322 (2007) 560; F. Li, |
| | | | J. Y. Sun, J. Y. Wang, S. L. |
| | | | Du, W. Y. Lu, M. Liu, et al., J. |
| | | | Control. Release 131 (2008) 77. |
| | PDGF receptor | PDGF | W. I. Hagens, A. Mattos, R. |
| | | | Greupink, A. de Jager- |
| | | | Krikken, C. Reker-Smit, A. |
| | | | van Loenen-Weemaes, et al., |
| | | | Pharm. Res. 24 (2007) 566. |
| | Scavenger | Human serum albumin | J. E. Adrian, K. Poelstra, G. L. |
| | receptor class A | | Scherphof, G. Molema, D. K. |
| | | | Meijer, C. Reker-Smit, et al., |
| | | | J. Hepatol. 44 (2006) 560; |
| | | | J. E. Adrian, J.A. Kamps, G. L. |
| | | | Scherphof, D. K. Meijer, A. M. |
| | | | van Loenen-Weemaes, C. |
| | | | Reker-Smit, et al., Biochim. |
| | | | Biophys. Acta 1768 (2007) |
| | | | 1430; J. E. Adrian, K. |
| | | | Poelstra, G. L. Scherphof, |
| | | | D. K. Meijer, A. M. van |
| | | | Loenen-Weemaes, C. Reker- |
| | | | Smit, et al., J. Pharmacol. |
| | | | Exp. Ther. 321 (2007) 536. |
| Hepatocytes | Asialoglycoprotein | Asialoorosomucoid | B. T. Kren, G. M. Unger, L. |
| | receptor | | Sjeklocha, A. A. Trossen, V. |
| | | | Korman, B. M. Diethelm- |
| | | | Okita, et al., J. Clin. Invest. |
| | | | 119 (2009) 2086. |
| | | Galactoside | F. Suriano, R. Pratt, J. P. |
| | | | Tan, N. Wiradharma, A. |
| | | | Nelson, Y. Y. Yang, et al., |
| | | | Biomaterials 31 (2010) 2637; |
| | | | T. Terada, M. Iwai, S. |
| | | | Kawakami, F. Yamashita, M. |
| | | | Hashida, J. Control. Release |
| | | | 111 (2006) 333. |
| | | Galactosamine | L. W. Seymour, D. R. Ferry, D. |
| | | | Anderson, S. Hesslewood, |
| | | | P. J. Julyan, R. Poyner, et al., |
| | | | J. Clin. Oncol. 20 (2002) |
| | | | 1668; Y. Cao, Y. Gu, H. Ma, |
| | | | J. Bai, L. Liu, P. Zhao, et al., |
| | | | Int. J. Biol. Macromol. 46 |
| | | | (2010) 245; Y. C. Wang, X. Q. |
| | | | Liu, T. M. Sun, M. H. Xiong, |
| | | | J. Wang, J. Control. Release |
| | | | 128 (2008) 32. |
| | | Asialofetuin | S. Diez, G. Navarro, I.C.T. |
| | | | de, J. Gene Med. 11 (2009) 38. |
| | | Sterylglucoside | X. R. Qi, W. W. Yan, J. Shi, |
| | | | World J. Gastroenterol. 11 |
| | | | (2005) 4947. |
| | | Lactose/lactobionic acid | Z. Xu, L. Chen, W. Gu, Y. |
| | | | Gao, L. Lin, Z. Zhang, et al., |
| | | | Biomaterials 30 (2009) 226; |
| | | | Y. Kato, H. Onishi, Y. |
| | | | Machida, Int. J. Pharm. 226 |
| | | | (2001) 93; K. W. Yang, X. R. |
| | | | Li, Z. L. Yang, P. Z. Li, F. |
| | | | Wang, Y. Liu, J. Biomed. |
| | | | Mater. Res. A 88 (2009) 140; |
| | | | Q. Wang, L. Zhang, W. Hu, |
| | | | Z. H. Hu, Y. Y. Bei, J. Y. Xu, et |
| | | | al., Nanomedicine 6 (2010) 371. |
| | | PVLA | C. S. Cho, A. Kobayashi, R. |
| | | | Takei, T. Ishihara, A. |
| | | | Maruyama, T. Akaike, |
| | | | Biomaterials 22 (2001) 45; Y. |
| | | | Watanabe, X. Liu, I. Shibuya, |
| | | | T. Akaike, J. Biomater. Sci. |
| | | | Polym. Ed. 11 (2000) 833. |
| | Scavenger | Apolipoprotein A-I | S. I. Kim, D. Shin, T. H. Choi, |
| | receptor class B | | J. C. Lee, G. J. Cheon, K. Y. |
| | type I | | Kim, et al., Mol. Ther. 15 |
| | | | (2007) 1145; S. I. Kim, D. |
| | | | Shin, H. Lee, B. Y. Ahn, Y. |
| | | | Yoon, M. Kim, J. Hepatol. 50 |
| | | | (2009) 479; M. Feng, Q. Cai, |
| | | | H. Huang, P. Zhou, Eur. J. |
| | | | Pharm. Biopharm. 68 (2008) 688. |
| | Plasma | Linoleic acid | S. J. Cheong, C. M. Lee, S. L. |
| | membrane fatty | | Kim, H. J. Jeong, E. M. Kim, |
| | acid binding | | E. H. Park, et al., Int. J. |
| | protein | | Pharm. 372 (2009) 169; C. M. |
| | (Putative) | | Lee, H. J. Jeong, S. L. Kim, |
| | | | E. M. Kim, D. W. Kim, S. T. |
| | | | Lim, et al., Int. J. Pharm. 371 |
| | | | (2009) 163. |
| | Glycyrrhizin | Glycyrrhizin | S. J. Mao, S. X. Hou, R. He, |
| | receptors | | L. K. Zhang, D. P. Wei, Y. Q. |
| | | | Bi, et al., World J. |
| | | | Gastroenterol. 11 (2005) 3075. |
| | Heparan sulfate | Acety- | K. J. Longmuir, S. M. Haynes, |
| | | ICKNEKKNKIERNNKLKQPP- | J. L. Baratta, N. Kasabwalla, |
| | | amide | R. T. Robertson, Int. J. |
| | | | Pharm. 382 (2009) 222. |
| | IL-6-receptor | Pre-S1 | R. Miyata, M. Ueda, H. Jinno, |
| | and/or | | T. Konno, K. Ishihara, N. |
| | immunoglobulin | | Ando, et al., Int. J. Cancer |
| | A binding | | 124 (2009) 2460. |
| | protein | | |
| | (Putative) | | |
| Macrophages | Macrophage | | Fakhrul Ahsan, Isabel P. |
| (including | receptors (Fc | | Rivas, Mansoor A. Khan, |
| Kupffer cells, | receptors, | | Ana I. Torres Suarez. |
| splenic | complement, | | Targeting to macrophages: |
| macrophages, | fibronectin | | role of physicochemical |
| etc. . .) | lipoprotein, | | properties of particulate |
| | mannosyl, | | carriers-liposomes and |
| | galactosyl) | | microspheres-on the |
| | | | phagocytosis by macrophages. |
| | | | Journal of Controlled Release |
| | | | 79 (2002) 29-40. |
| | Passive | Positively charged and large | R. A. Schwendener, P. A. |
| | targeting | sized Liposomes | Lagocki, Y. E. Rahman, The |
| | | | effects of charge and size on |
| | | | the interaction of unilamellar |
| | | | liposomes with |
| | | | macrophages, Biochim. |
| | | | Biophys. Acta 7721992, pp. |
| | | | 195-200. (1984) 93-101; |
| | | | Y. E. Rahman, E. A. Cernry, |
| | | | K. R. Patel, E. H. Lau, B. J. |
| | | | Wright, Differential uptake of |
| | | | liposomes varying in size and |
| | | | lipid composition by |
| | | | parenchymal and kupffer |
| | | | cells of mouse liver, Life Sci. |
| | | | 31 (1982) 2061-2071. |
| | | Inclusion of negatively | Fakhrul Ahsan, Isabel P. |
| | | charged phospholipids such | Rivas, Mansoor A. Khan, |
| | | as phosphatidylserine and | Ana I. Torres Suarez. |
| | | phosphatidylglycerol | Targeting to macrophages: |
| | | Peptide grafted liposomes | role of physicochemical |
| | | Hydrophobic and large sized | properties of particulate |
| | | polymeric microspheres | carriers-liposomes and |
| | | Coating of polymeric | microspheres-on the |
| | | microspheres with opsonic | phagocytosis by macrophages. |
| | | materials (gamma-globulin, | Journal of Controlled Release |
| | | human fibronectin, bovine | 79 (2002) 29-40. |
| | | tuftsin, and gelatin) | |

T Cells

T cells (or T lymphocytes) are a type of lymphocyte that play a central role in cell-mediated immunity. They can be distinguished from other lymphocytes, such as B cells and natural killer cells (NK cells), by the presence of a T cell receptor (TCR) on the cell surface.

The T cells used in the present invention may be used for adoptive T cell transfer.

The term “adoptive T cell transfer”, as used herein, refers to the administration of a T cell population to a patient. A T cell may be isolated from a subject and then genetically modified and cultured in vitro (ex vivo) in order to express a TCR or chimeric antigen receptor (CAR) before being administered to a patient.

Adoptive cell transfer may be allogenic or autologous.

By “autologous cell transfer” it is to be understood that the starting population of cells is obtained from the same subject as that to which the T cell population is administered.

Autologous transfer is advantageous as it avoids problems associated with immunological incompatibility and is available to subjects irrespective of the availability of a genetically matched donor.

By “allogeneic cell transfer” it is to be understood that the starting population of cells is obtained from a different subject as that to which the T cell population is administered.

Preferably, the donor will be genetically matched to the subject to which the cells are administered to minimise the risk of immunological incompatibility. Alternatively, the donor may be mismatched and unrelated to the patient.

Suitable doses of transduced cell populations are such as to be therapeutically and/or prophylactically effective. The dose to be administered may depend on the subject and condition to be treated, and may be readily determined by a skilled person.

The T cell may be derived from a T cell isolated from a patient. The T cell may be part of a mixed cell population isolated from the subject, such as a population of peripheral blood lymphocytes (PBL). T cells within the PBL population may be activated by methods known in the art, such as using anti-CD3 and/or anti-CD28 antibodies or cell sized beads conjugated with anti-CD3 and/or anti-CD28 antibodies.

The T cell may be a CD4+ helper T cell or a CD8+ cytotoxic T cell. The T cell may be in a mixed population of CD4+ helper T cells/CD8+ cytotoxic T cells. Polyclonal activation, for example using anti-CD3 antibodies optionally in combination with anti-CD28 antibodies may trigger the proliferation of CD4+ and CD8+ T cells.

A T cell may be isolated from the subject to which the population of T cells is to be adoptively transferred. In this respect, the cell may be made by isolating a T cell from a subject, optionally activating the T cell, optionally transferring a TCR- or CAR-encoding gene to the cell ex vivo. Subsequent immunotherapy of the subject may then be carried out by adoptive transfer of the population of cells.

Alternatively the T cell may be derived from a stem cell, such as a haemopoietic stem cell (HSC). Gene transfer into HSCs does not lead to TCR expression at the cell surface as stem cells do not express CD3 molecules. However, when stem cells differentiate into lymphoid precursors that migrate to the thymus, the initiation of CD3 expression leads to the surface expression of the TCR in thymocytes.

An advantage of this approach is that the mature T cells, once produced, express only an introduced TCR and little or no endogenous TCR chains, because the expression of the introduced TCR chains suppresses rearrangement of endogenous TCR gene segments to form functional TCR alpha and beta genes. A further benefit is that the gene-modified stem cells are a continuous source of mature T cells with the desired antigen specificity. Accordingly, the vector as defined herein may be used in combination with a gene-modified stem cell, preferably a gene-modified hematopoietic stem cell, which, upon differentiation, produces a T cell.

Other approaches known in the art may be used to reduce, limit, prevent, silence, or abrogate expression of endogenous genes in the cells of the present invention or cells prepared by the methods of the present invention.

The term “disrupting”, as used herein, refers to reducing, limiting, preventing, silencing or abrogating expression of a gene. The skilled person is able to use any method known in the art to disrupt an endogenous gene, e.g. any suitable method for genome editing, gene silencing, gene knock-down or gene knock-out.

For example, an endogenous gene may be disrupted with an artificial nuclease. An artificial nuclease is, e.g. an artificial restriction enzyme engineered to selectively target a specific polynucleotide sequence (e.g. encoding a gene of interest) and induce a double strand break in said polynucleotide sequence. Typically, the double strand break (DSB) will be repaired by error-prone non-homologous end joining (NHEJ) thereby resulting in the formation of a non-functional polynucleotide sequence, which may be unable to express an endogenous gene.

In some embodiments, the artificial nuclease is selected from the group consisting of zinc finger nucleases (ZFNs), transcription activator-like effector nucleases (TALENs) and CRISPR/Cas (e.g. CRISPR/Cas9).

T Cell Receptor (TCR)

During antigen processing, antigens are degraded inside cells and then carried to the cell surface by major histocompatibility complex (MHC) molecules. T cells are able to recognise this peptide:MHC complex at the surface of the antigen presenting cell. There are two different classes of MHC molecules: MHC I and MHC II, each class delivers peptides from different cellular compartments to the cell surface.

A T cell receptor (TCR) is a molecule found on the surface of T cells that is responsible for recognising antigens bound to MHC molecules. The TCR heterodimer consists of an alpha (α) and beta (β) chain in around 95% of T cells, whereas around 5% of T cells have TCRs consisting of gamma (γ) and delta (δ) chains.

Engagement of the TCR with antigen and MHC results in activation of the T lymphocyte on which the TCR is expressed through a series of biochemical events mediated by associated enzymes, co-receptors and specialised accessory molecules.

Each chain of the TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end.

The variable domain of both the TCR α chain and β chain have three hypervariable or complementarity determining regions (CDRs). CDR3 is the main CDR responsible for recognising processed antigen, although CDR1 of the alpha chain has also been shown to interact with the N-terminal part of the antigenic peptide, whereas CDR1 of the beta chain interacts with the C-terminal part of the peptide. CDR2 is thought to recognise the MHC molecule.

The constant domain of the TCR domain consists of short connecting sequences in which a cysteine residue forms a disulfide bond, making a link between the two chains.

The TCR used in the present invention may have one or more additional cysteine residues in each of the α and β chains such that the TCR may comprise two or more disulphide bonds in the constant domains.

The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3- and ζ-chains, together with the TCR, form what is known as the T cell receptor complex.

The signal from the T cell complex is enhanced by simultaneous binding of the MHC molecules by a specific co-receptor. For helper T cells, this co-receptor is CD4 (specific for class II MHC); whereas for cytotoxic T cells, this co-receptor is CD8 (specific for class I MHC). The co-receptor allows prolonged engagement between the antigen presenting cell and the T cell and recruits essential molecules (e.g. LCK) inside the cell involved in the signalling of the activated T lymphocyte.

Accordingly, the term “T cell receptor” (TCR), as used herein, refers to a molecule capable of recognising a peptide when presented by an MHC molecule. The molecule may be a heterodimer of two chains α and β (or optionally γ and δ) or it may be a single chain TCR construct.

The TCR used in the present invention may be a hybrid TCR comprising sequences derived from more than one species. For example, it has surprisingly been found that murine TCRs are more efficiently expressed in human T cells than human TCRs. The TCR may therefore comprise human variable regions and murine constant regions.

A disadvantage of this approach is that the murine constant sequences may trigger an immune response, leading to rejection of the transferred T cells. However, the conditioning regimens used to prepare patients for adoptive T cell therapy may result in sufficient immunosuppression to allow the engraftment of T cells expressing murine sequences.

The portion of the TCR that establishes the majority of the contacts with the antigenic peptide bound to the major histocompatibility complex (MHC) is the complementarity determining region 3 (CDR3), which is unique for each T cell clone. The CDR3 region is generated upon somatic rearrangement events occurring in the thymus and involving non-contiguous genes belonging to the variable (V), diversity (D, for β and δ chains) and joining (J) genes. Furthermore, random nucleotides inserted/deleted at the rearranging loci of each TCR chain gene greatly increase diversity of the highly variable CDR3 sequence. Thus, the frequency of a specific CDR3 sequences in a biological sample indicates the abundance of a specific T cell population. The great diversity of the TCR repertoire in healthy human beings provides a wide range protection towards a variety of foreign antigens presented by MHC molecules on the surface of antigen presenting cells. In this regard, it is of note that theoretically up to 10¹⁵ different TCRs can be generated in the thymus.

T cell receptor diversity is focused on CDR3 and this region is primarily responsible for antigen recognition.

TCRs specific for a hepatitis virus antigen may be generated easily by the person skilled in the art using any method known in the art.

Suitable hepatitis virus antigens include hepatitis B virus large envelope protein; hepatitis B virus middle envelope protein; hepatitis B virus small envelope protein; hepatitis B virus core protein; and hepatitis B virus polymerase.

For example, hepatitis virus antigen-specific TCRs maybe identified by the TCR gene capture method of Linnemann et al. (Nature Medicine 19: 1534-1541 (2013)). Briefly, this method uses a high-throughput DNA-based strategy to identify TCR sequences by the capture and sequencing of genomic DNA fragments encoding the TCR genes and may be used to identify hepatitis virus antigen-specific TCRs.

Improved TCR Expression and Reduced TCR Mispairing

Increasing the supply of CD3 molecules may increase TCR expression, for example, in a cell that has been modified to express the TCRs of the present invention. Accordingly, the T cell may be modified (e.g. using a vector) to comprise one or more genes encoding CD3-gamma, CD3-delta, CD3-epsilon and/or CD3-zeta. In some embodiments, the T cell comprises a gene encoding CD3-zeta. The T cell may comprise a gene encoding CD8. The vector encoding such genes may encode a selectable marker or a suicide gene, to increase the safety profile of the genetically engineered cell. The genes may be linked by self-cleaving sequences, such as the 2A self-cleaving sequence.

Alternatively one or more separate vectors encoding a CD3 gene may be provided for co-transfer to a T cell simultaneously, sequentially or separately with one or more vectors encoding TCRs.

The transgenic TCR may be expressed in a T cell used in the present invention to alter the antigen specificity of the T cell. TCR-transduced T cells express at least two TCR alpha and two TCR beta chains. While the endogenous TCR alpha/beta chains form a receptor that is self-tolerant, the introduced TCR alpha/beta chains form a receptor with defined specificity for the given target antigen.

However, TCR gene therapy requires sufficient expression of transferred (i.e. transgenic) TCRs as the transferred TCR might be diluted by the presence of the endogenous TCR, resulting in suboptimal expression of the tumor specific TCR. Furthermore, mispairing between endogenous and introduced chains may occur to form novel receptors, which might display unexpected specificities for self-antigens and cause autoimmune damage when transferred into patients.

Hence, several strategies have been explored to reduce the risk of mispairing between endogenous and introduced TCR chains. Mutations of the TCR alpha/beta interface is one strategy currently employed to reduce unwanted mispairing. For example, the introduction of a cysteine in the constant domains of the alpha and beta chain allows the formation of a disulfide bond and enhances the pairing of the introduced chains while reducing mispairing with wild type chains.

Accordingly, the TCRs used in the present invention may comprise one or more mutations at the α chain/β chain interface, such that when the α chain and the β chain are expressed in a T cell, the frequency of mispairing between said chains and endogenous TCR a and β chains is reduced. In some embodiments, the one or more mutations introduce a cysteine residue into the constant region domain of each of the α chain and the β chain, wherein the cysteine residues are capable of forming a disulphide bond between the α chain and the β chain.

Another strategy to reduce mispairing relies on the introduction of polynucleotide sequences encoding siRNA and designed to limit the expression of the endogenous TCR genes (Okamoto S. (2009) Cancer research 69: 9003-9011).

Accordingly, the vector or polynucleotide encoding the TCRs used in the present invention may comprise one or more siRNA or other agents aimed at limiting or abrogating the expression of the endogenous TCR genes.

It is also possible to combine artificial nucleases, such as zinc finger nucleases (ZFNs), transcription activator-like effector nucleases (TALENs) or CRISPR/Cas systems, designed to target the constant regions of the endogenous TCR genes (TRAC and/or TRBC), to obtain the permanent disruption of the endogenous TCR alpha and/or beta chain genes, thus allowing full expression of the tumor specific TCR and thus reducing or abrogating the risk of TCR mispairing. This process, known as the TCR gene editing proved superior to TCR gene transfer in vitro and in vivo (Provasi E. et al. (2012) Nature Medicine 18: 807-15).

In addition, the genome editing technology allows targeted integration of a expression cassette, comprising a polynucleotide encoding a TCR used in the present invention, and optionally one or more promoter regions and/or other expression control sequences, into an endogenous gene disrupted by the artificial nucleases (Lombardo A. (2007) Nature Biotechnology 25: 1298-1306).

Another strategy developed to increase expression of transgenic TCRs and to reduce TCR mispairing consists in “murinisation,” which replaces the human TCR α and TCR β constant regions (e.g. the TRAC, TRBC1 and TRBC2 regions) by their murine counterparts. Murinisation of TCR constant regions is described in, for example, Sommermeyer et al. (2010) J Immunol 184: 6223-6231. Accordingly, the TCR used in the present invention may be murinised.

Chimeric Antigen Receptor (CAR)

CARs comprise an extracellular ligand binding domain, most commonly a single chain variable fragment of a monoclonal antibody (scFv) linked to intracellular signaling components, most commonly CD3 alone or combined with one or more costimulatory domains. A spacer is often added between the extracellular antigen-binding domain and the transmembrane moiety to optimise the interaction with the target.

A CAR for use in the present invention may comprise:
(i) an antigen-specific targeting domain;
(ii) a transmembrane domain;
(iii) optionally at least one costimulatory domain; and
(iv) an intracellular signaling domain.

Preferably, the antigen-specific targeting domain comprises an antibody or fragment thereof, more preferably a single chain variable fragment.

Preferably, the antigen-specific targeting domain targets a hepatitis virus antigen.

In some embodiments, the hepatitis virus antigen is selected from the group consisting of hepatitis B virus large envelope protein; hepatitis B virus middle envelope protein; hepatitis B virus small envelope protein; hepatitis B virus core protein; and hepatitis B virus polymerase.

Examples of transmembrane domains include a transmembrane domain of a zeta chain of a T cell receptor complex, CD28 and CD8a.

Examples of costimulatory domains include costimulating domains from CD28, CD137 (4-1BB), CD134 (OX40), DapIO, CD27, CD2, CD5, ICAM-1, LFA-1, Lck, TNFR-I, TNFR-II, Fas, CD30 and CD40.

In some embodiments, the costimulatory domain is a costimulating domain from CD28.

Examples of intracellular signaling domains include human CD3 zeta chain, FcyRIII, FcsRI, a cytoplasmic tail of a Fc receptor and an immunoreceptor tyrosine-based activation motif (ITAM) bearing cytoplasmic receptors.

The term “chimeric antigen receptor” (“CAR” or “CARs”), as used herein, refers to engineered receptors which can confer an antigen specificity onto cells (for example, T cells such as naive T cells, central memory T cells, effector memory T cells or combinations thereof). CARs are also known as artificial T cell receptors, chimeric T cell receptors or chimeric immunoreceptors.

The antigen-specific targeting domain provides the CAR with the ability to bind to the target antigen of interest. The antigen-specific targeting domain preferably targets an antigen of clinical interest against which it would be desirable to trigger an effector immune response.

The antigen-specific targeting domain may be any protein or peptide that possesses the ability to specifically recognise and bind to a biological molecule (e.g. a hepatitis virus antigen). The antigen-specific targeting domain includes any naturally occurring, synthetic, semi-synthetic or recombinantly produced binding partner for a biological molecule of interest.

Illustrative antigen-specific targeting domains include antibodies or antibody fragments or derivatives, extracellular domains of receptors, ligands for cell surface molecules/receptors, or receptor binding domains thereof.

In preferred embodiments, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) and a camelid antibody (VHH).

In preferred embodiments, the binding domain is a single chain antibody (scFv). The scFv may be, for example, a murine, human or humanised scFv.

The term “complementarity determining region” (“CDR”) with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs.

“Heavy chain variable region” (“VH”) refers to the fragment of the heavy chain of an antibody that contains three CDRs interposed between flanking stretches known as framework regions, which are more highly conserved than the CDRs and form a scaffold to support the CDRs.

“Light chain variable region” (“VL”) refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain.

“Single-chain Fv antibody” (“scFv”) refers to an engineered antibody consisting of a light chain variable region and a heavy chain variable region connected to one another directly or via a peptide linker sequence.

Antibodies that specifically bind a target antigen can be prepared using methods well known in the art. Such methods include phage display, methods to generate human or humanised antibodies, or methods using a transgenic animal or plant engineered to produce human antibodies. Phage display libraries of partially or fully synthetic antibodies are available and can be screened for an antibody or fragment thereof that can bind to the target molecule.

Phage display libraries of human antibodies are also available. Once identified, the amino acid sequence or polynucleotide sequence coding for the antibody can be isolated and/or determined.

The CAR used in the present invention may also comprise one or more co-stimulatory domains. This domain may enhance cell proliferation, cell survival and development of memory cells.

Each co-stimulatory domain comprises the co-stimulatory domain of any one or more of, for example, members of the TNFR super family, CD28, CD137 (4-1BB), CD134 (OX40), DapIO, CD27, CD2, CDS, ICAM-1, LFA-1, Lck, TNFR-1, TNFR-II, Fas, CD30, CD40 or combinations thereof. Co-stimulatory domains from other proteins may also be used with the CAR used in the present invention.

The CAR used in the present invention may also comprise an intracellular signaling domain. This domain may be cytoplasmic and may transduce the effector function signal and direct the cell to perform its specialised function. Examples of intracellular signaling domains include, but are not limited to, ζ chain of the T cell receptor or any of its homologues (e.g. ƒ chain, FcεR1γ and β chains, MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T cell transduction, such as CD2, CD5 and CD28. The intracellular signaling domain may be human CD3 zeta chain, FcyRIII, FcsRI, cytoplasmic tails of Fc receptors, immunoreceptor tyrosine-based activation motif (ITAM) bearing cytoplasmic receptors or combinations thereof.

The CAR used in the present invention may also comprise a transmembrane domain. The transmembrane domain may comprise the transmembrane sequence from any protein which has a transmembrane domain, including any of the type I, type II or type III transmembrane proteins. The transmembrane domain of the CAR used in the present invention may also comprise an artificial hydrophobic sequence. The transmembrane domains of the CARs used in the present invention may be selected so as not to dimerise. Examples of transmembrane (TM) regions used in CAR constructs are: 1) The CD28 TM region (Pule et al, Mol Ther, 2005, November; 12(5):933-41; Brentjens et al, CCR, 2007, Sep. 15; 13(18 Pt 1):5426-35; Casucci et al, Blood, 2013, Nov. 14; 122(20):3461-72.); 2) The OX40 TM region (Pule et al, Mol Ther, 2005, November; 12(5):933-41); 3) The 41BB TM region (Brentjens et al, CCR, 2007, Sep. 15; 13(18 Pt 1):5426-35); 4) The CD3 zeta TM region (Pule et al, Mol Ther, 2005, November; 12(5):933-41; Savoldo B, Blood, 2009, Jun. 18; 113(25):6392-402.); 5) The CD8a TM region (Maher et al, Nat Biotechnol, 2002, January; 20(1):70-5.; Imai C, Leukemia, 2004, April; 18(4):676-84; Brentjens et al, CCR, 2007, Sep. 15; 13(18 Pt 1):5426-35; Milone et al, Mol Ther, 2009, August; 17(8):1453-64.).

Variants, Derivatives, Analogues, Homologues and Fragments

In addition to the specific proteins and nucleotides mentioned herein, the invention also encompasses variants, derivatives, analogues, homologues and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 96% or 97% or 98% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60).

However, for some applications, it is preferred to use the GCG Bestf it program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

Method of Treatment

All references herein to treatment include curative, palliative and prophylactic treatment. The treatment of mammals, particularly humans, is preferred. Both human and veterinary treatments are within the scope of the invention.

In some embodiments, the method of treatment provides the interleukin to the liver of a subject.

In some embodiments, the method of treatment provides the interleukin to hepatocytes.

Pharmaceutical Compositions and Injected Solutions

Although the agents for use in the invention can be administered alone, they will generally be administered in admixture with a pharmaceutical carrier, excipient or diluent, particularly for human therapy.

The medicaments, for example vectors or cells, of the invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used. In some cases, serum albumin may be used in the composition.

For injection, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

Administration

In some embodiments, the interleukin is administered to a subject locally.

In preferred embodiments, the interleukin is administered to a subject's liver.

In some embodiments, the vector, cell or composition is administered to a subject locally.

In preferred embodiments, the vector, cell or composition is administered to a subject's liver.

The term “systemic delivery” or “systemic administration” as used herein means that the agent of the invention is administered into the circulatory system, for example to achieve broad distribution of the agent. In contrast, topical or local administration restricts the delivery of the agent to a localised area e.g. the liver.

As used herein, the term “agent” may refer, for example, to the vector, cell or pharmaceutical composition of the invention.

In some embodiments, the interleukin is administered simultaneously, sequentially or separately in combination with a population of T cells. In some embodiments, the vector is administered simultaneously, sequentially or separately in combination with a population of T cells. In some embodiments, the T cells express a chimeric antigen receptor (CAR) or a T cell receptor (TCR), which binds to a hepatitis virus antigen.

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

Dosage

The skilled person can readily determine an appropriate dose of an agent of the invention to administer to a subject. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is human.

The invention is further described by the following numbered paragraphs:
1. A vector comprising a nucleotide sequence encoding an interleukin which binds to IL-2 receptor (IL-2R), wherein the vector is adapted for liver-specific expression of the nucleotide sequence.
2. The vector of paragraph 1, wherein the nucleotide sequence is operably linked to one or more expression control sequences for liver-specific expression.
3. The vector of paragraph 1 or 2, wherein the nucleotide sequence is operably linked to one or more miR-142, miR-155 and/or miR-223 target sequences, preferably wherein the nucleotide sequence is operably linked to one or more miR-142 target sequences.
4. The vector of any preceding paragraph, wherein the vector comprises 2, 3 or 4 miR-142, miR-155 and/or miR-223 target sequences operably linked to the nucleotide sequence.
5. The vector of any preceding paragraph, wherein the vector comprises a hepatocyte-specific promoter and/or enhancer operably linked to the nucleotide sequence.
6. The vector of paragraph 5, wherein the hepatocyte-specific promoter is selected from the group consisting of an ET promoter, albumin promoter, transthyretin promoter, alpha1-antitrypsin promoter and apoE/alpha1-antitrypsin promoter, preferably wherein the promoter is an ET promoter.
7. The vector of any preceding paragraph, wherein the interleukin is selected from the group consisting of IL-2, IL-7 and IL-15, preferably wherein the interleukin is IL-2.
8. The vector of any preceding paragraph, wherein the vector comprises: (a) a nucleotide sequence encoding IL-2; (b) a nucleotide sequence encoding IL-7; and/or (c) a nucleotide sequence encoding IL-15, preferably wherein each of (a)-(c) is operably linked to one or more expression control sequences for liver-specific expression.
9. The vector of any preceding paragraph, wherein the vector is a viral vector and/or an RNA vector.
10. The vector of any preceding paragraph, wherein the vector is a retroviral, lentiviral, adenoviral or adeno-associated viral (AAV) vector, preferably a lentiviral vector.
11. The vector of any preceding paragraph, wherein the vector is an integration-defective lentiviral vector (IDLV).
12. The vector of any preceding paragraph, wherein the vector is in the form of a viral vector particle.
13. The vector of any one of paragraphs 1-9, wherein the vector is in the form of a liposome or lipid nanoparticle, preferably wherein the vector is an RNA vector.
14. A composition or kit comprising two or more vectors selected from the group consisting of:
   (a) the vector of any preceding paragraph, wherein the vector comprises a nucleotide sequence encoding IL-2;
   (b) the vector of any preceding paragraph, wherein the vector comprises a nucleotide sequence encoding IL-7; and
   (c) the vector of any preceding paragraph, wherein the vector comprises a nucleotide sequence encoding IL-15,
   wherein at least two vectors are selected from different groups (a), (b) or (c).
15. A pharmaceutical composition comprising the vector of any one of paragraphs 1-13 or the composition of paragraph 14, and a pharmaceutically-acceptable carrier, diluent or excipient.
16. The pharmaceutical composition of paragraph 15, wherein the pharmaceutical composition further comprises a population of T cells, preferably wherein the T cells express a chimeric antigen receptor (CAR) or a T cell receptor (TCR), which binds to a hepatitis virus antigen.
17. A vector, composition or kit according to any one of paragraphs 1-16 for use in treating or preventing a viral liver infection and/or hepatocellular carcinoma.
18. The vector, composition or kit for use according to paragraph 17, wherein the viral liver infection is a hepatitis virus infection, preferably a chronic hepatitis virus infection.
19. The vector, composition or kit for use according to paragraph 17 or 18, wherein the viral liver infection is an hepatitis B virus (HBV) and/or hepatitis C virus (HCV) infection, preferably an HBV infection.
20. The vector, composition or kit for use according to any one of paragraphs 17-19, wherein the vector or composition is locally administered to a subject, preferably to a subject's liver.
21. The vector, composition or kit for use according to any one of paragraphs 17-20, wherein the vector or composition is administered as part of an adoptive T cell therapy.
22. The vector, composition or kit for use according to any one of paragraphs 17-21, wherein the vector is administered simultaneously, separately or sequentially with a population of T cells, preferably wherein the T cells express a chimeric antigen receptor (CAR) or a T cell receptor (TCR), which binds to a hepatitis virus antigen.
23. A product comprising (a) the vector or composition of any one of paragraphs 1-16; and (b) a population of T cells, as a combined preparation for simultaneous, separate or sequential use in therapy, preferably wherein the T cells express a chimeric antigen receptor (CAR) or a T cell receptor (TCR), which binds to a hepatitis virus antigen.
24. The product for use according to paragraph 23, wherein the product is for use in treating or preventing a viral liver infection or hepatocellular carcinoma.
25. The product for use according to paragraph 24, wherein the viral liver infection is a hepatitis virus infection, preferably a chronic hepatitis virus infection.
26. The product for use according to paragraph 24 or 25, wherein the viral liver infection is an hepatitis B virus (HBV) and/or hepatitis C virus (HCV) infection, preferably an HBV infection.
27. The product for use according to any one of paragraphs 23-26, wherein the vector and/or population of T cells is locally administered to a subject, preferably to a subject's liver.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

Results

Dynamic and Molecular Characterisation of HBV-Specific CD8+ T Cells Undergoing Priming in a Non-Inflamed Liver Expressing HBV Antigens

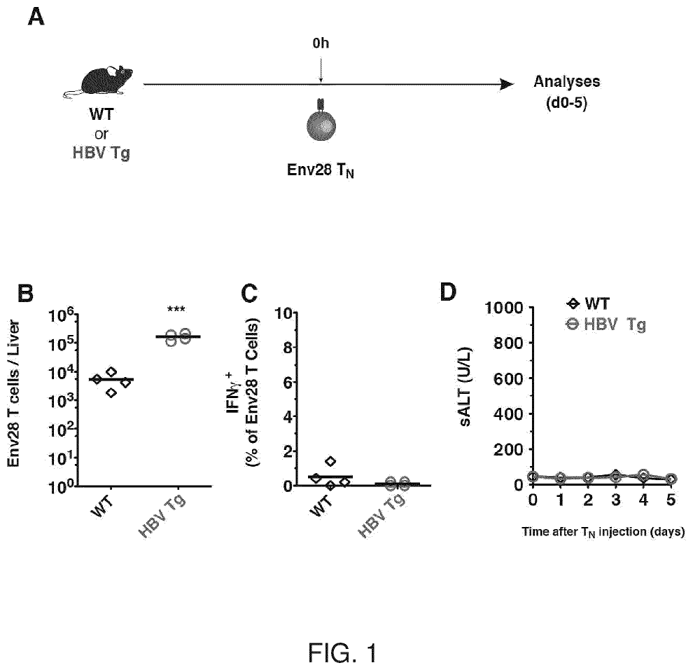

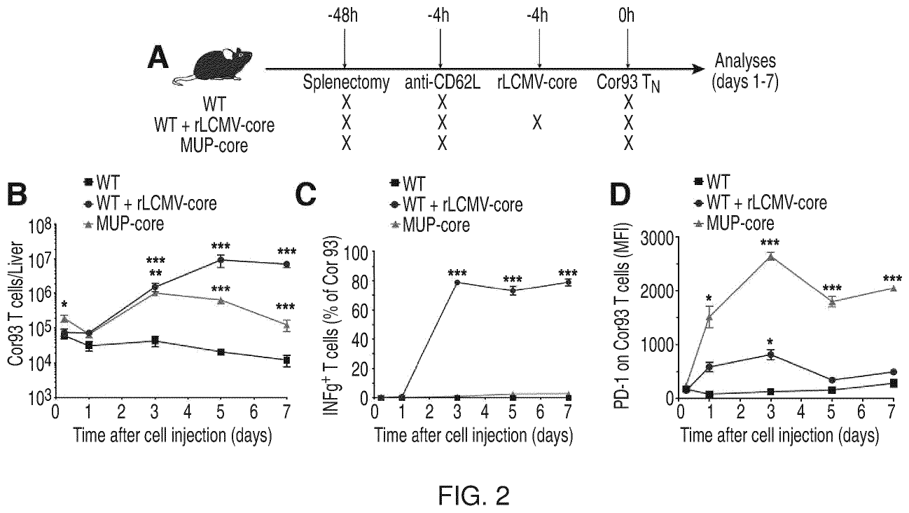

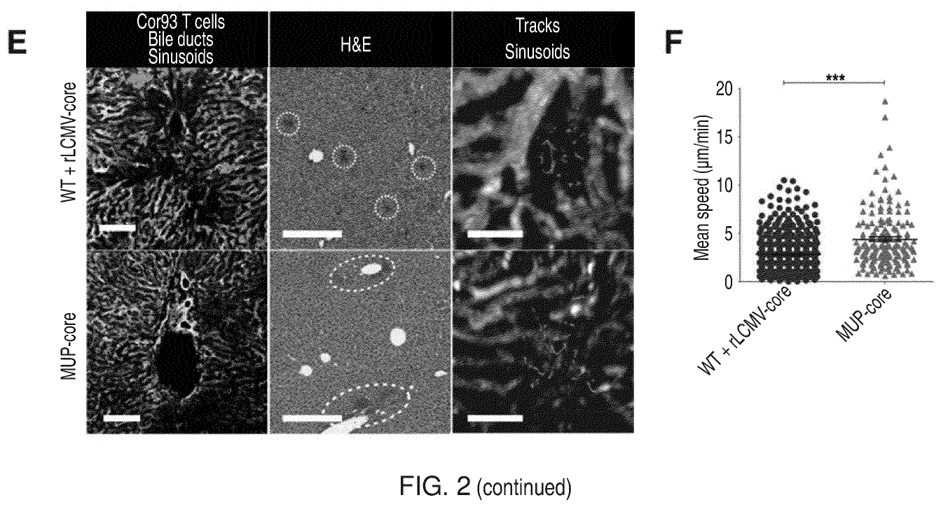

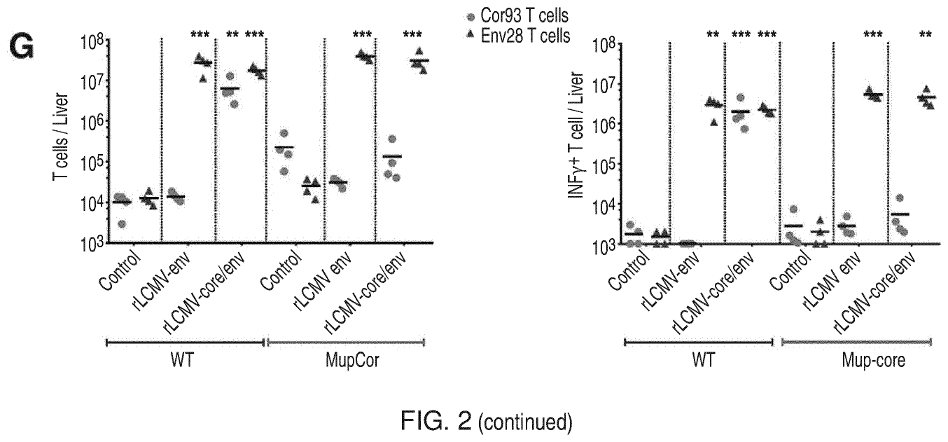

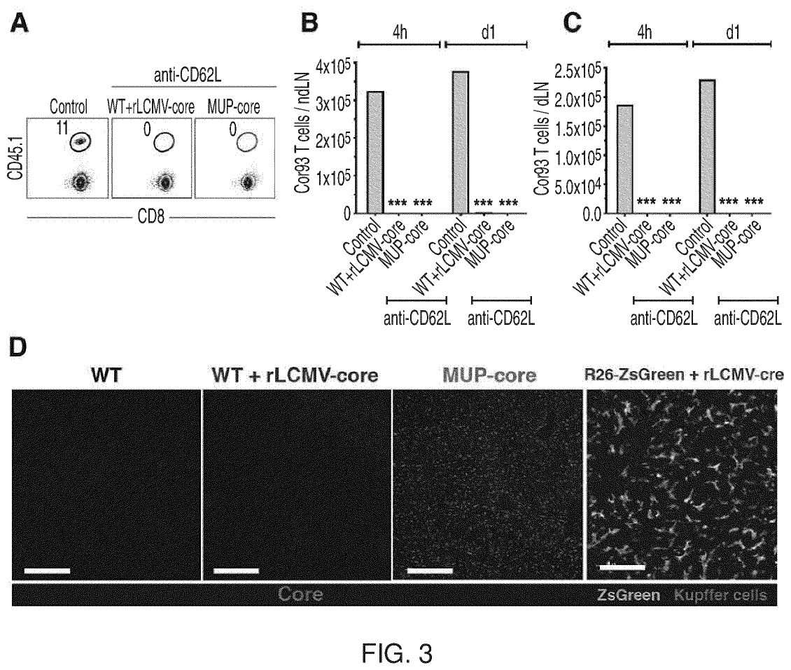

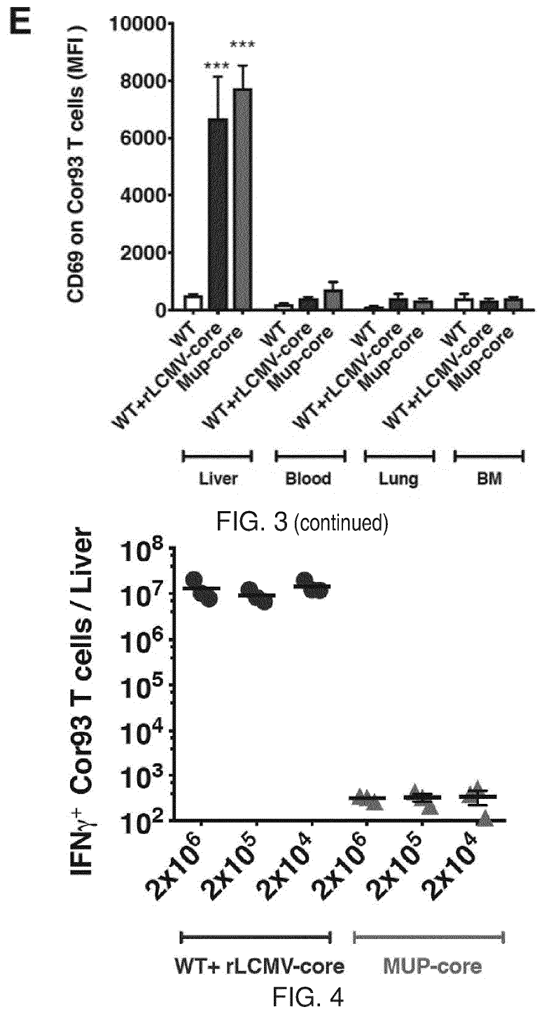

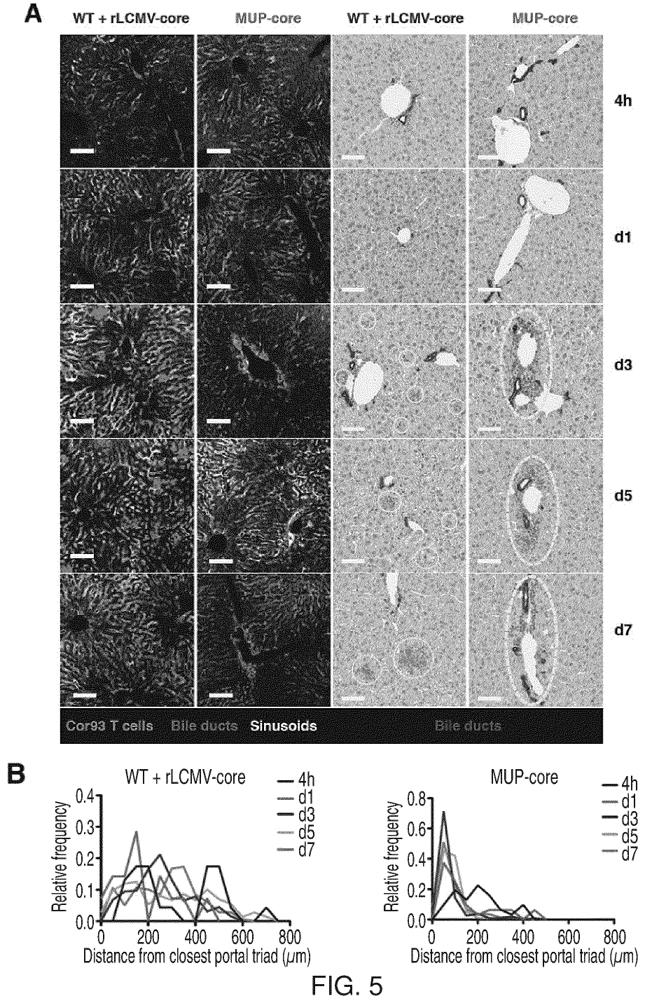

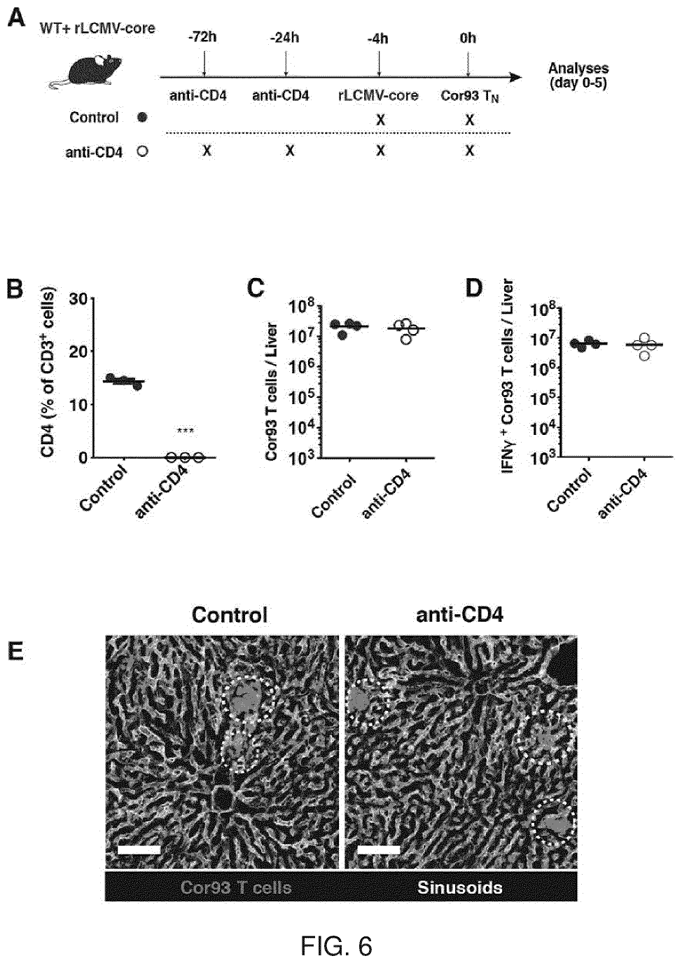

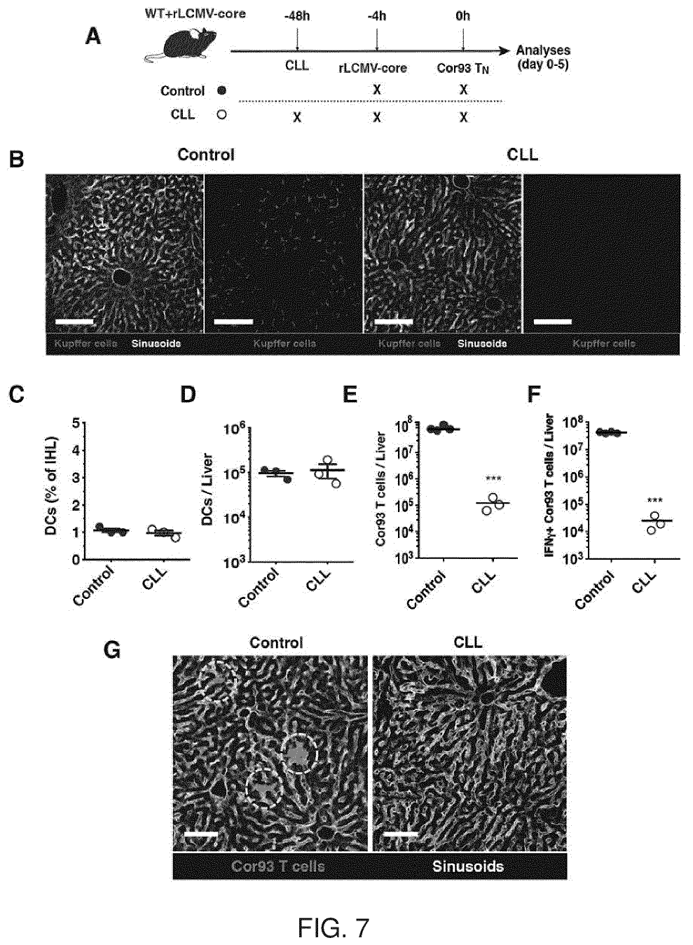

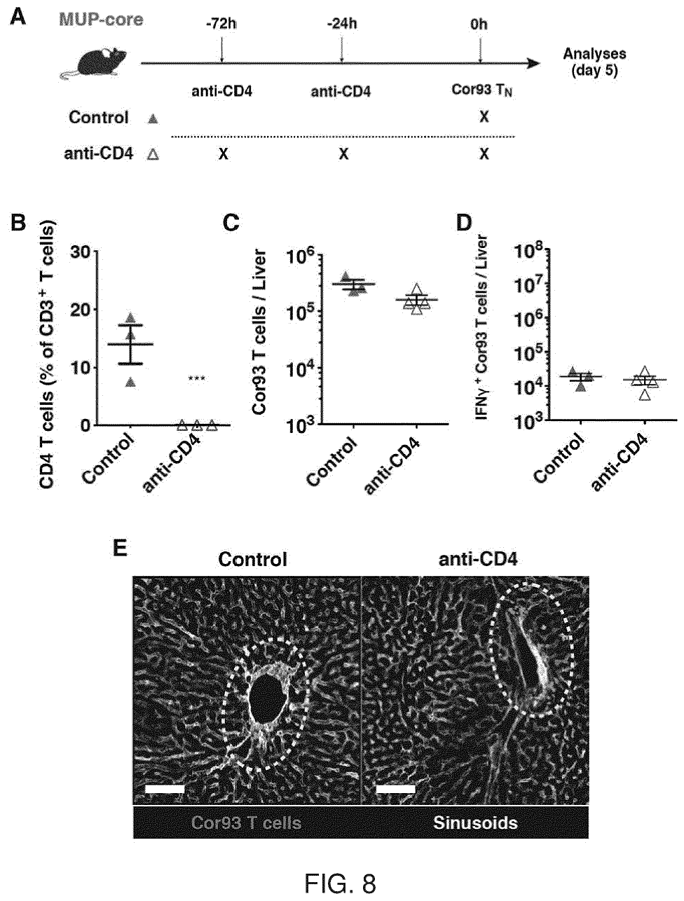

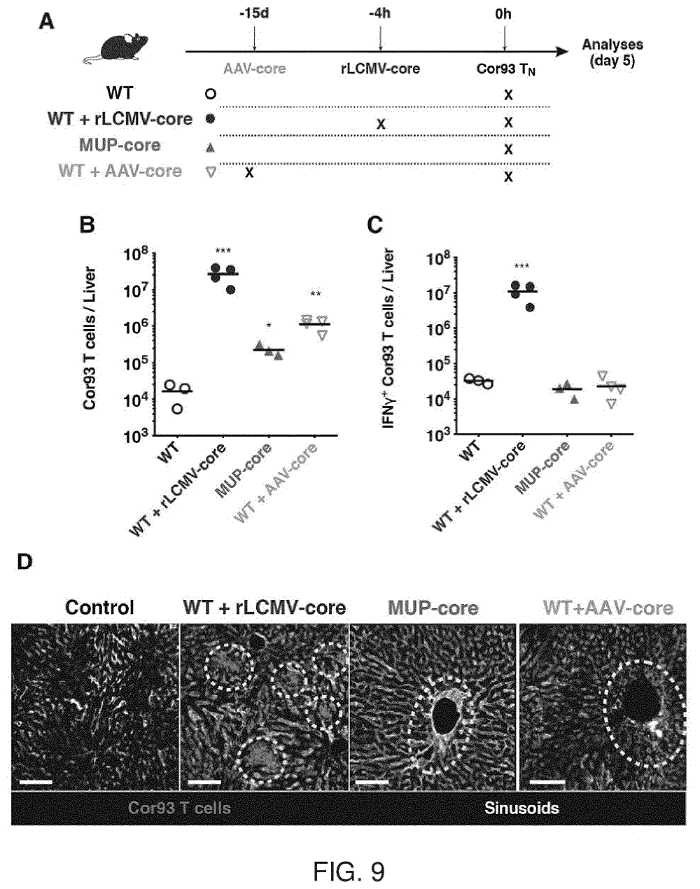

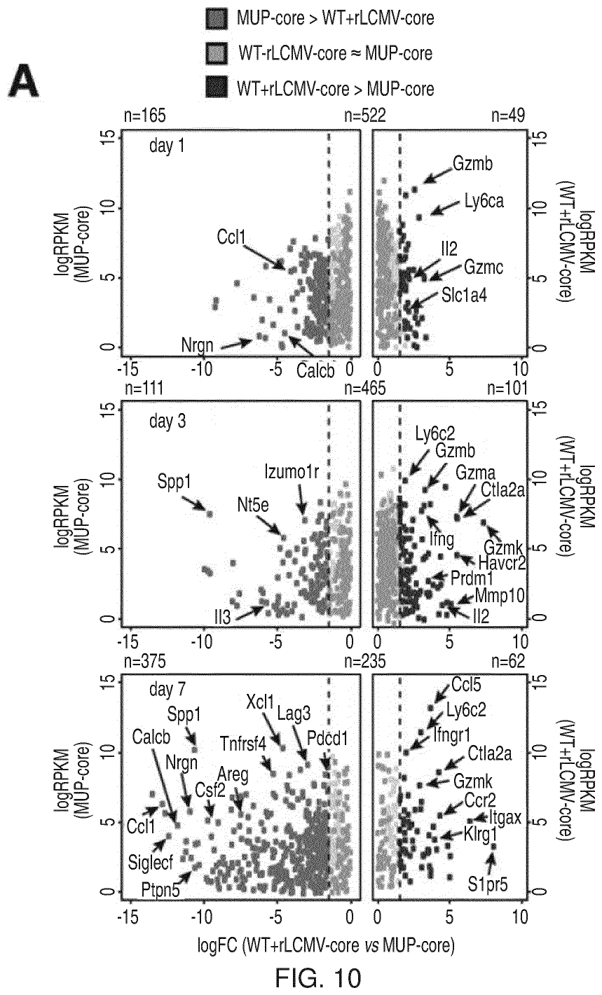

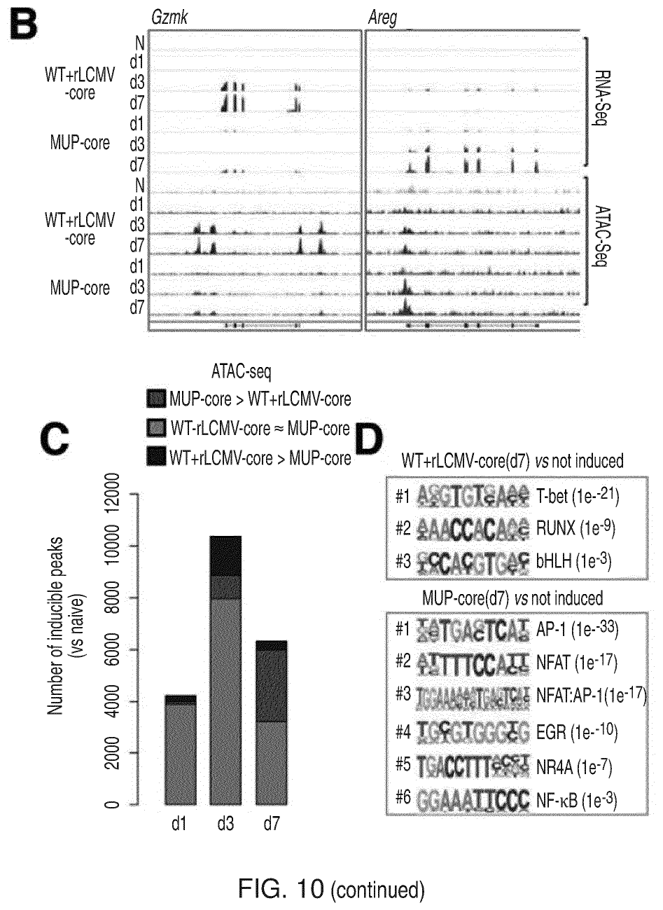

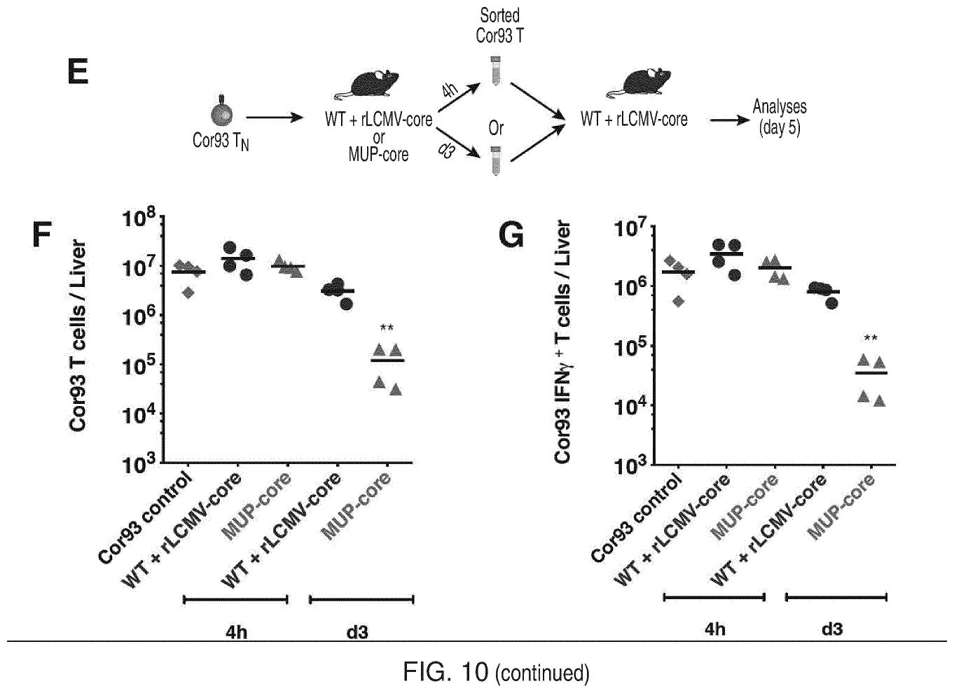

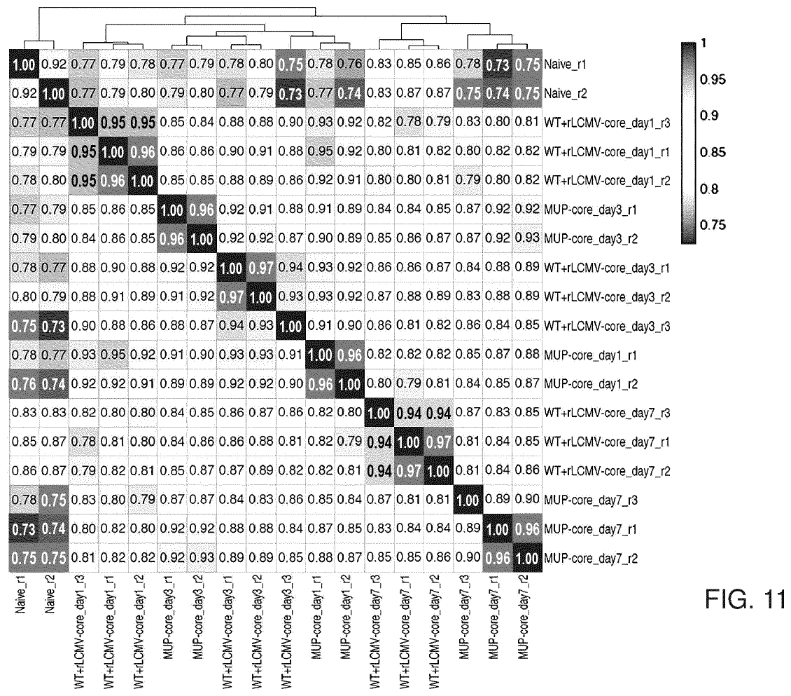

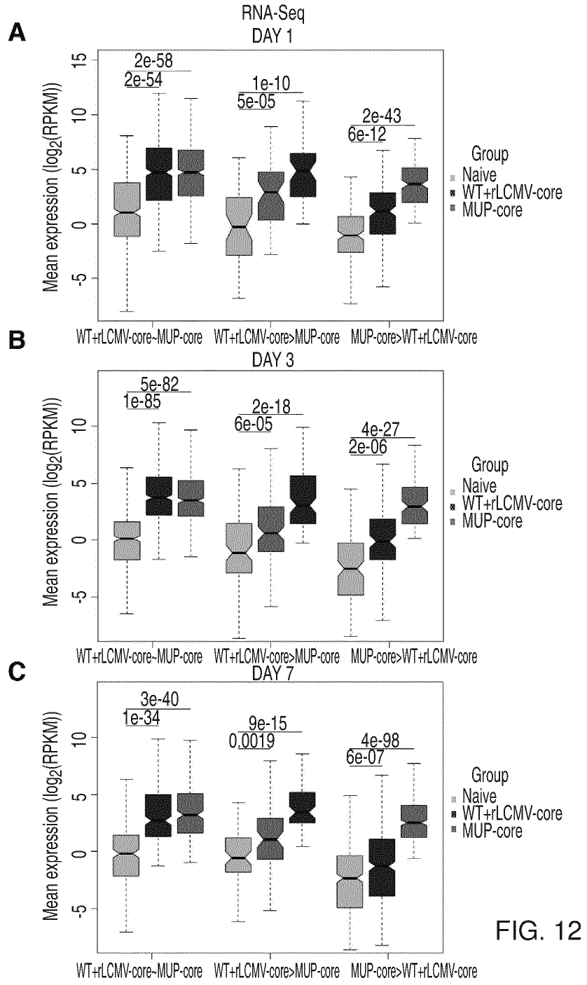

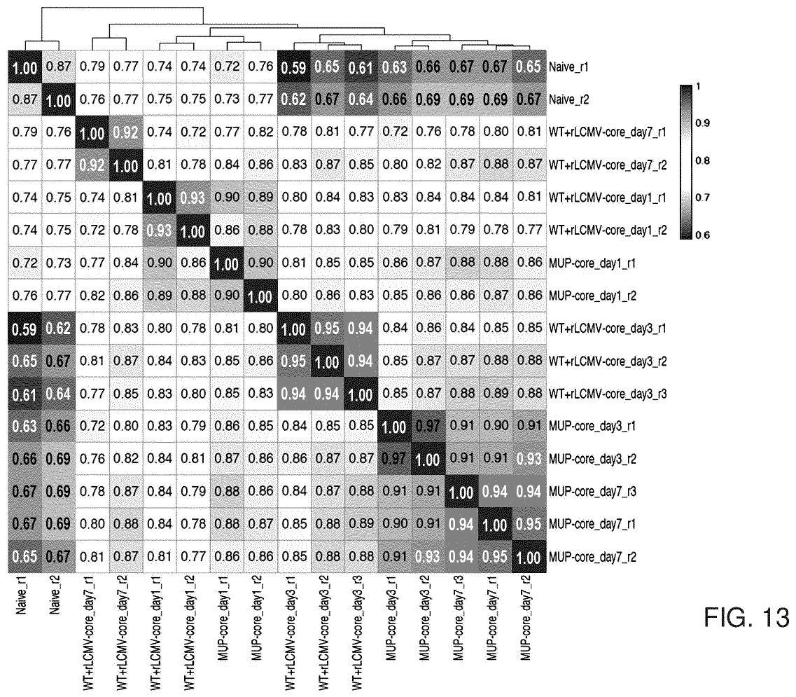

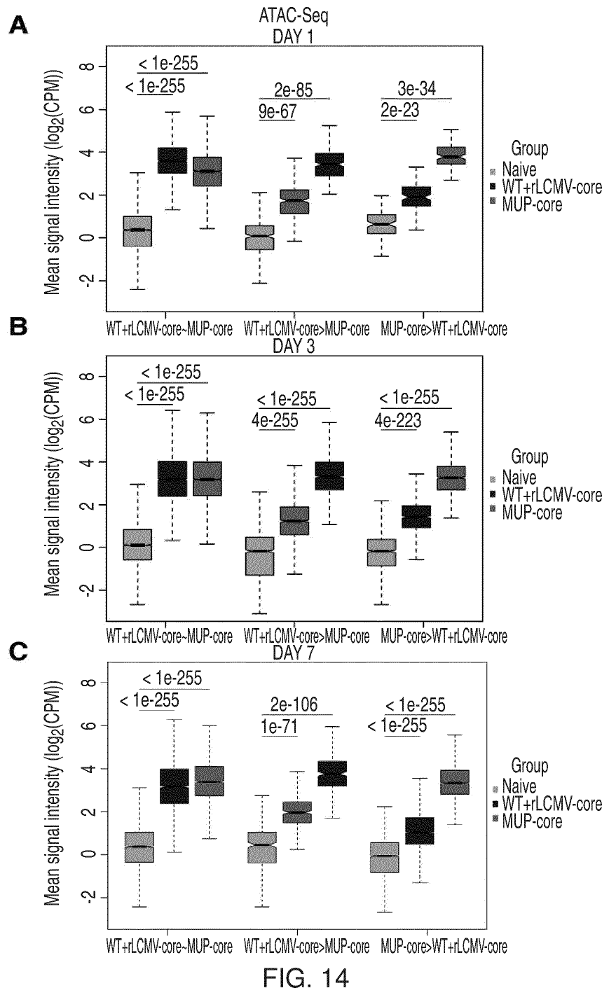

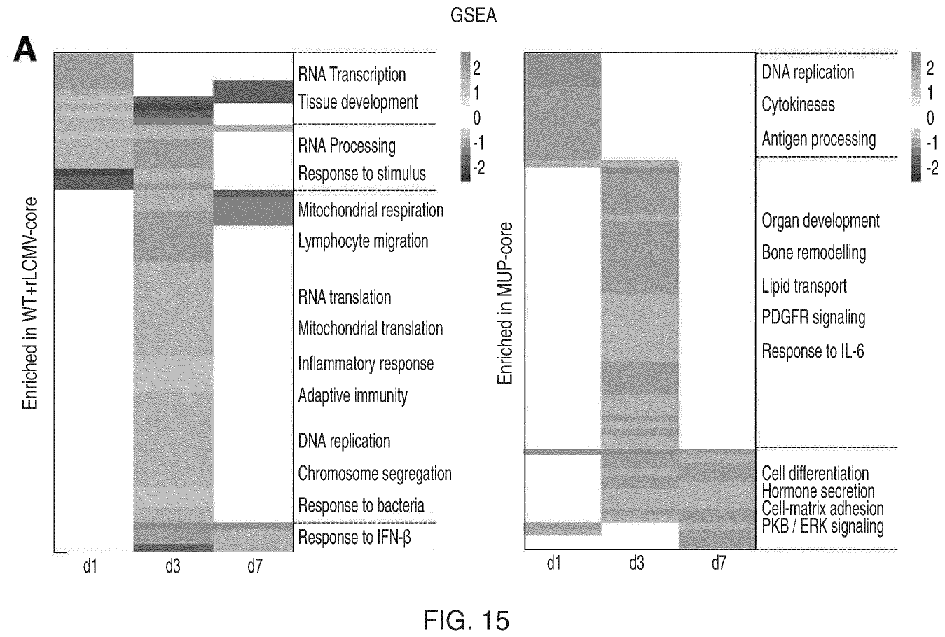

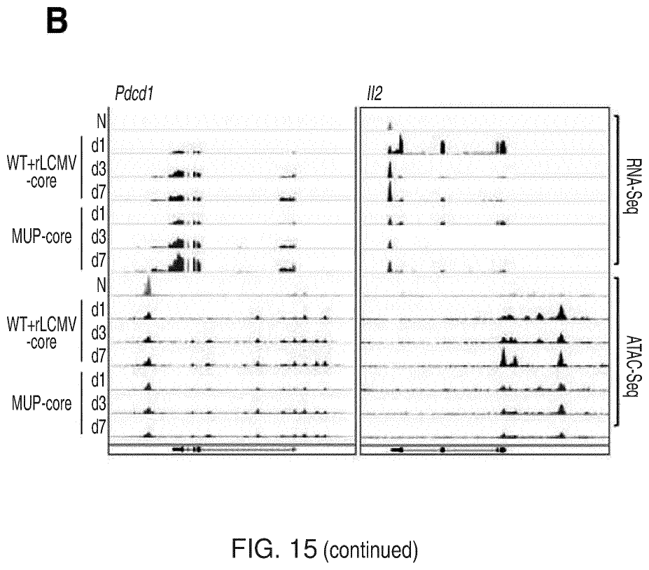

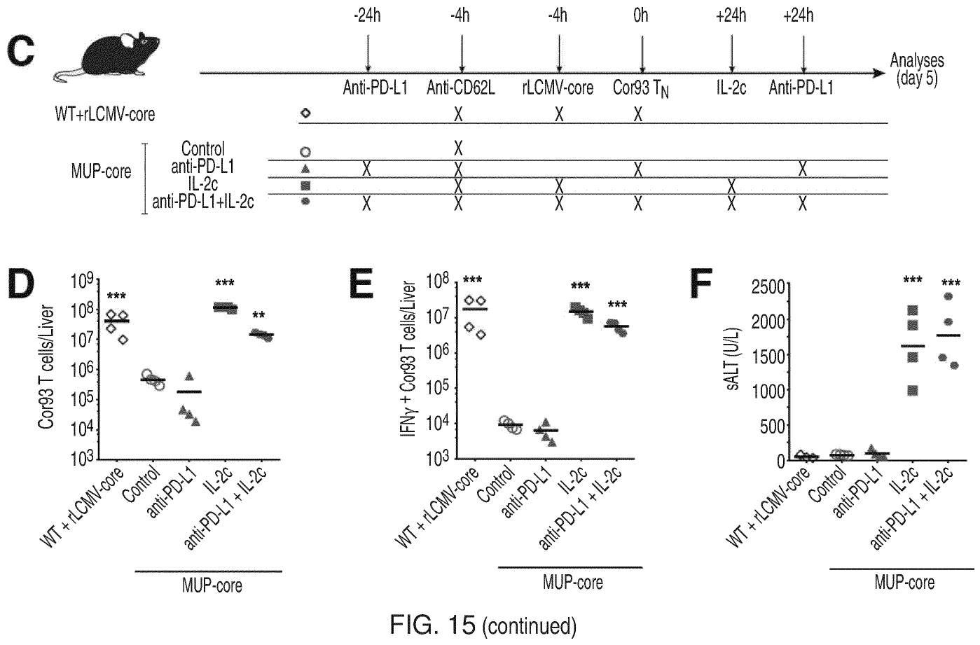

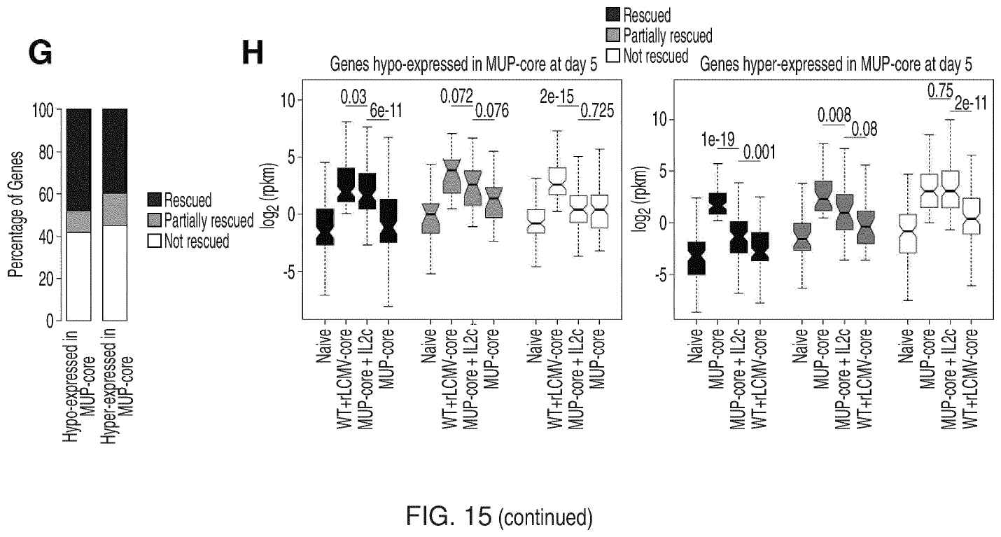

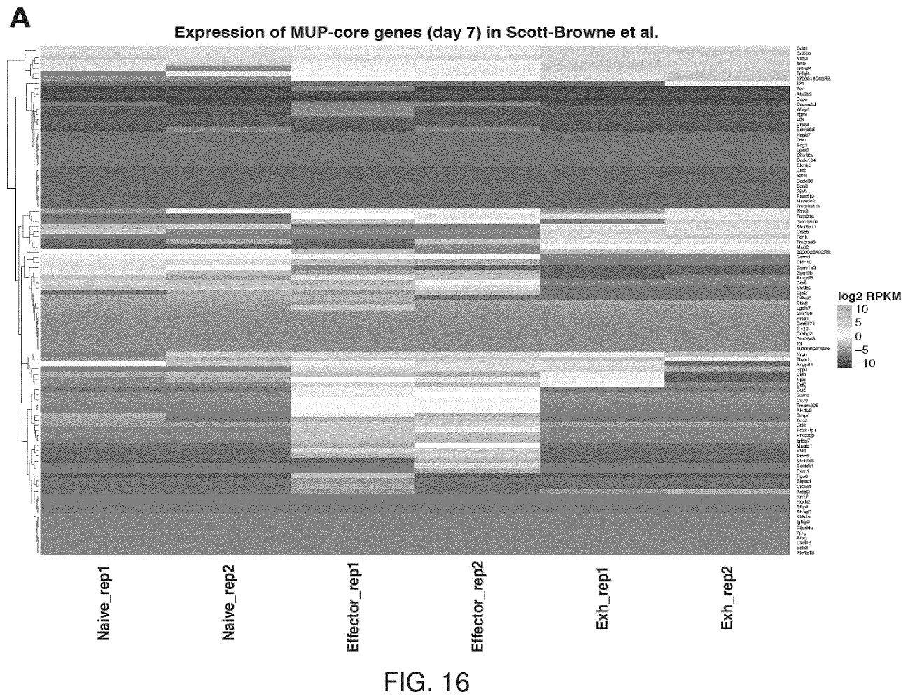

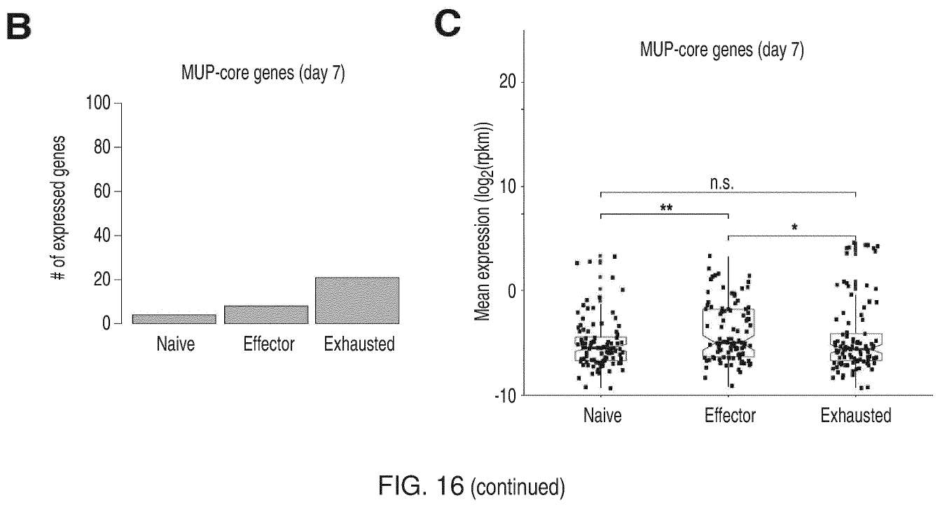

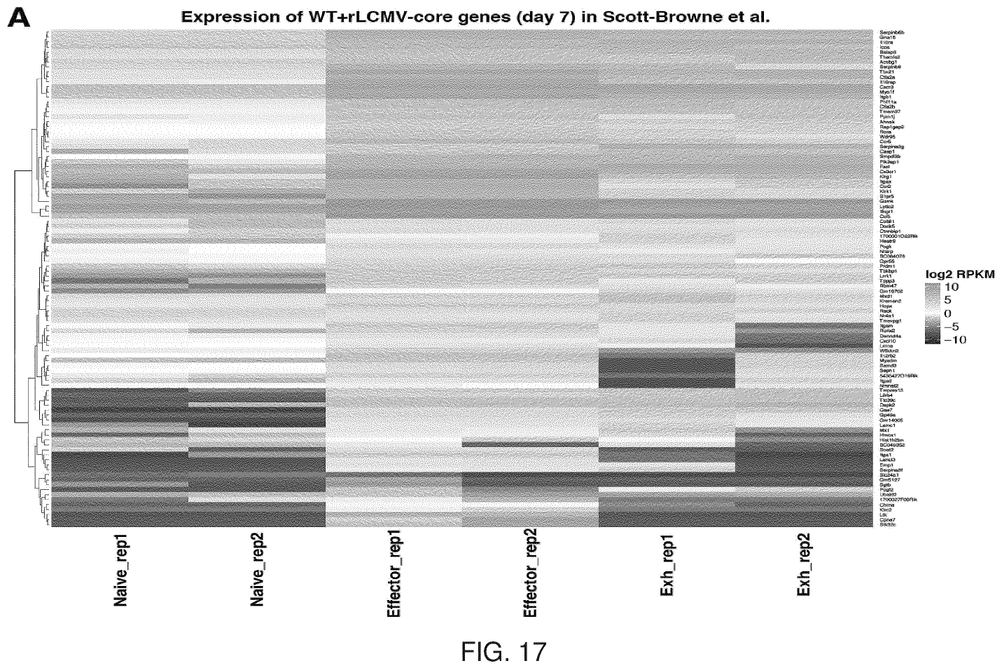

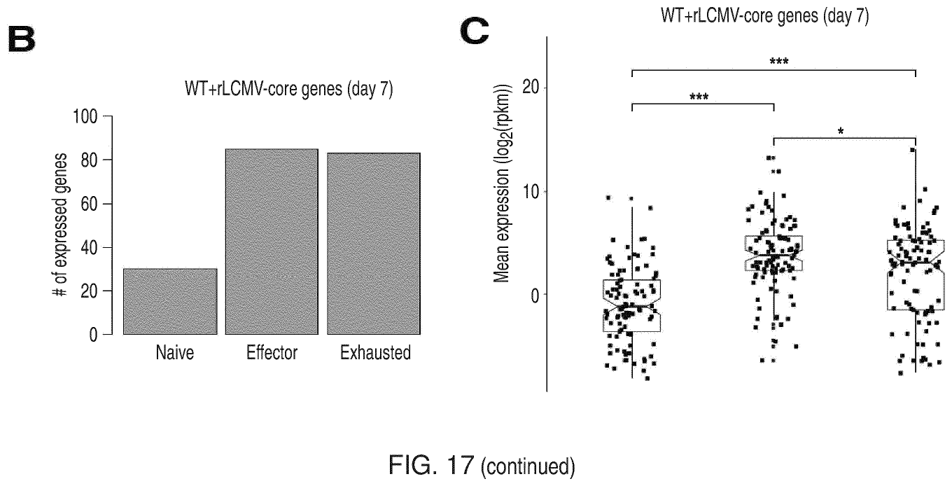

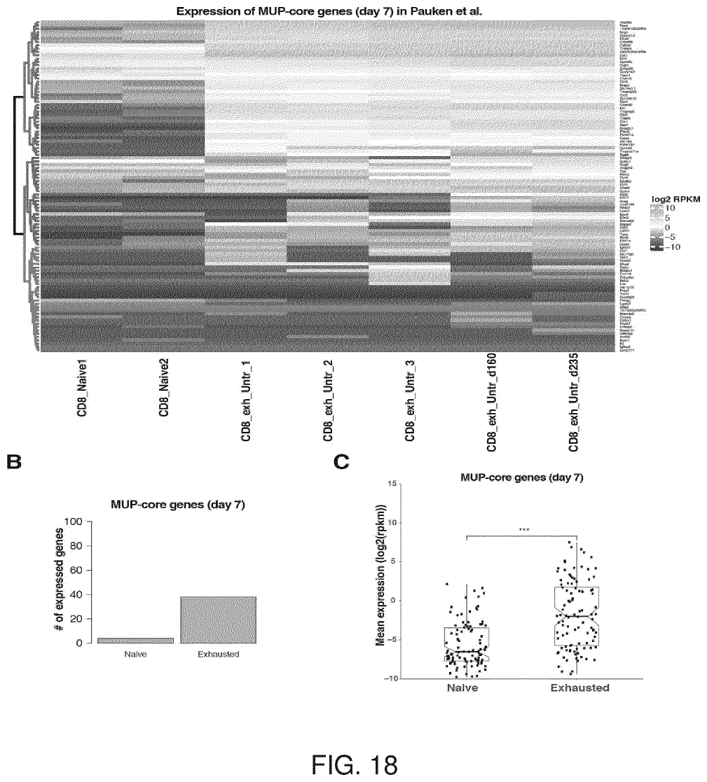

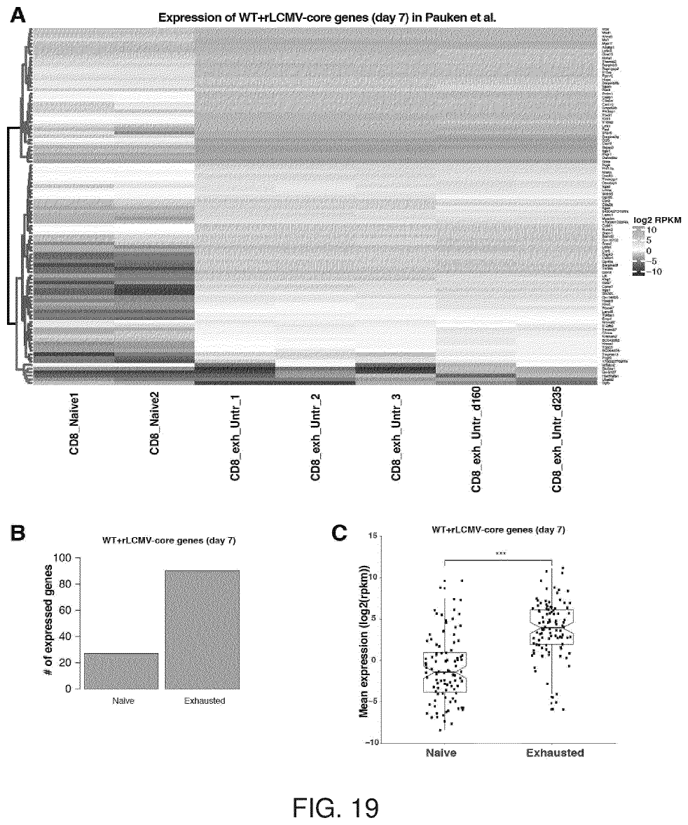

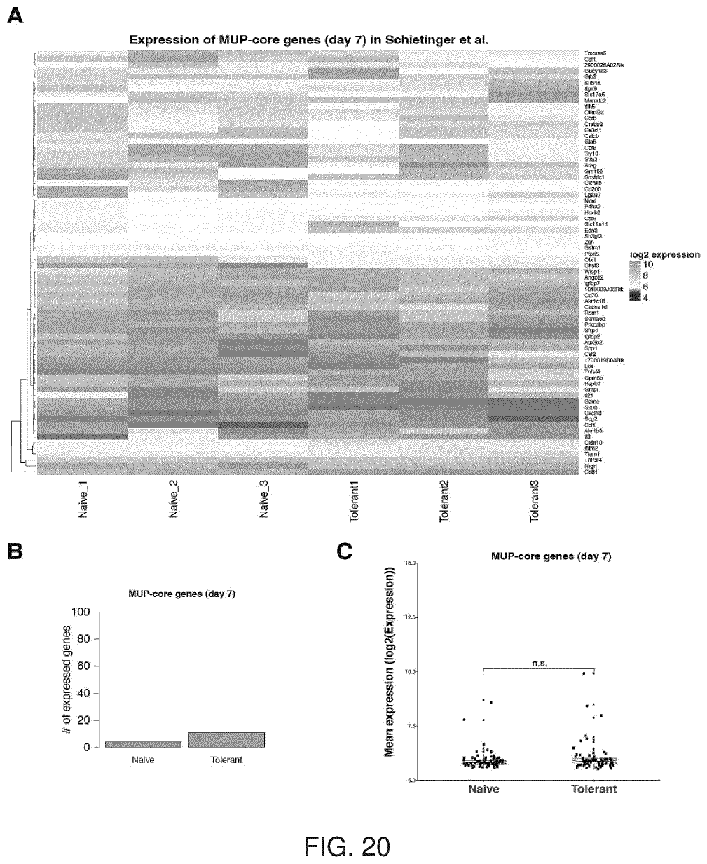

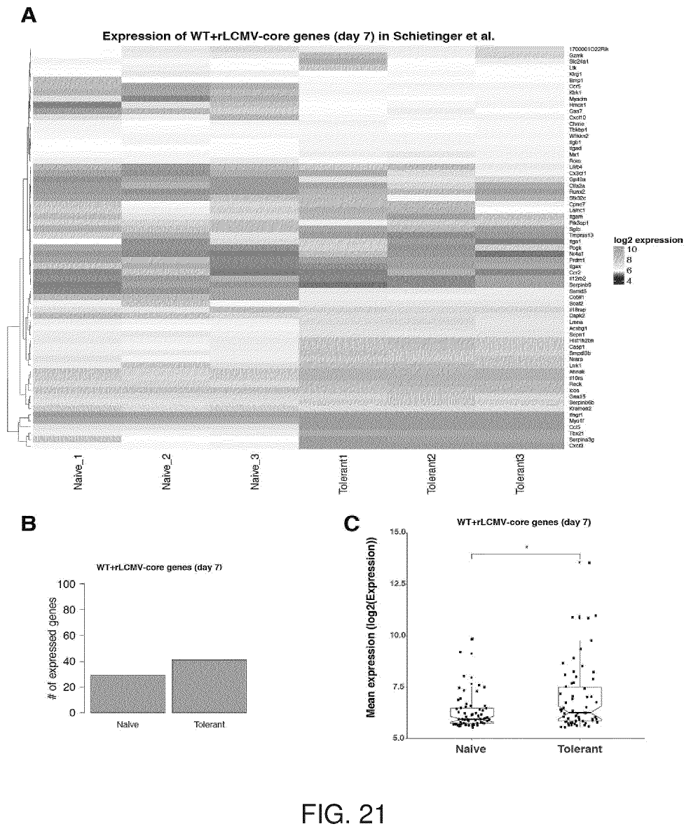

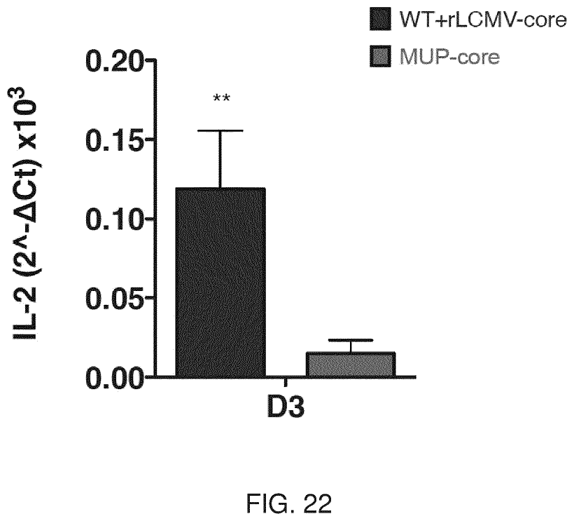

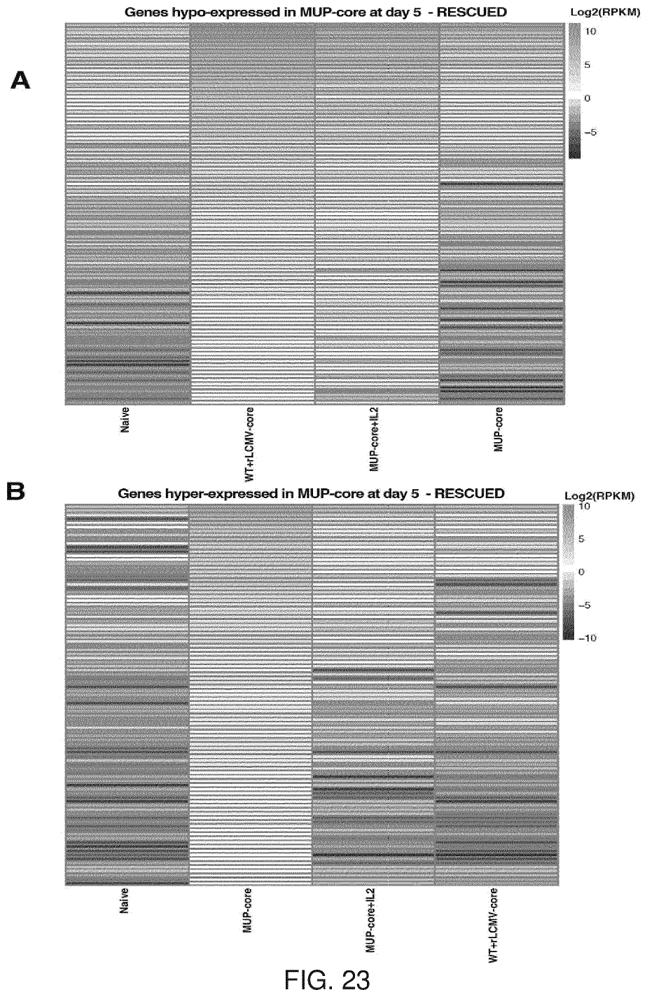

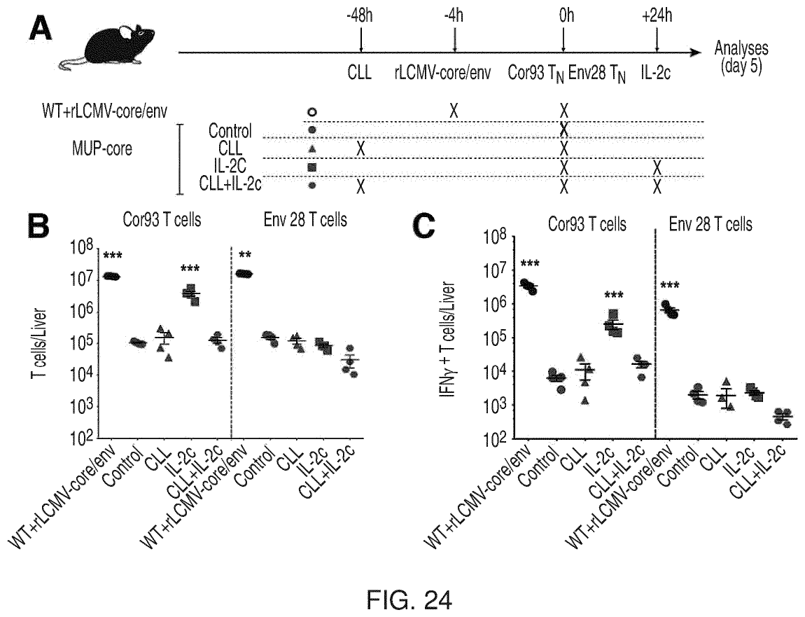

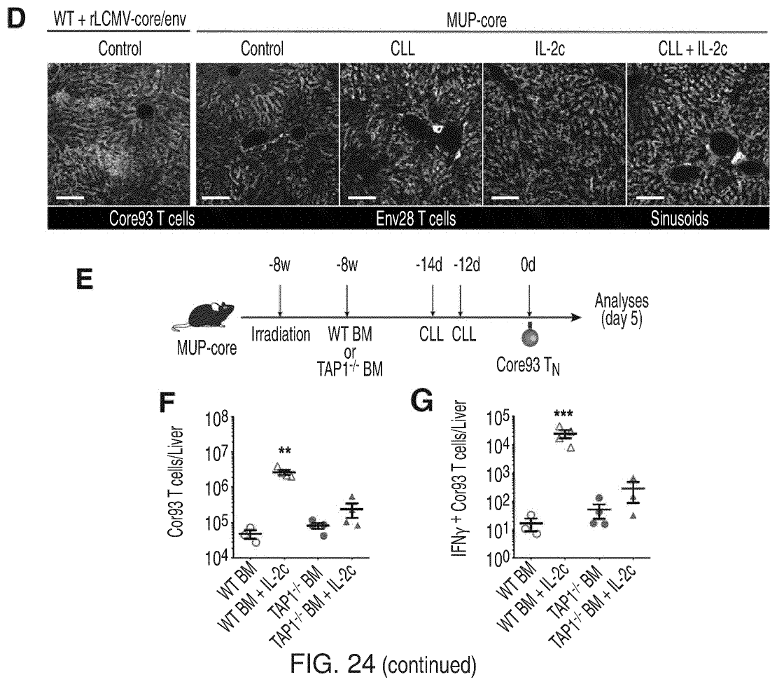

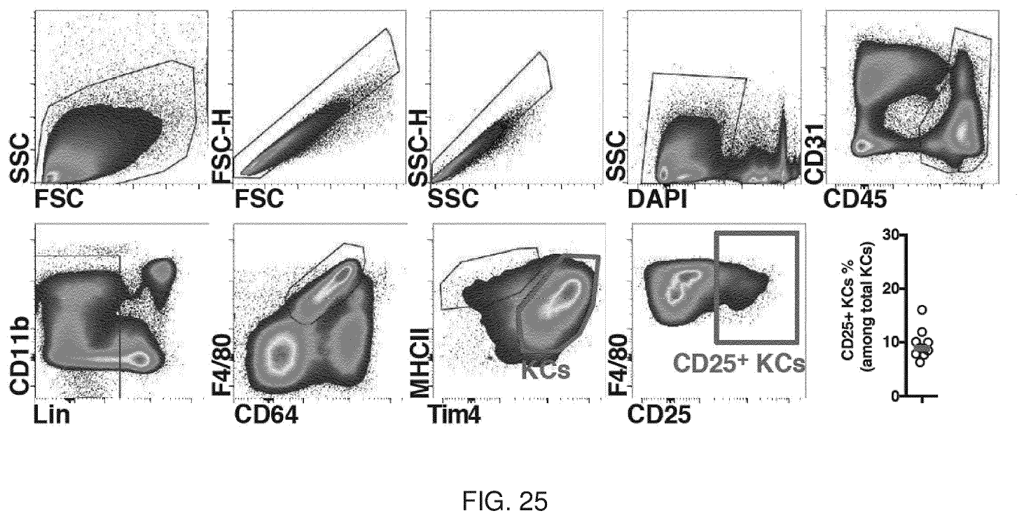

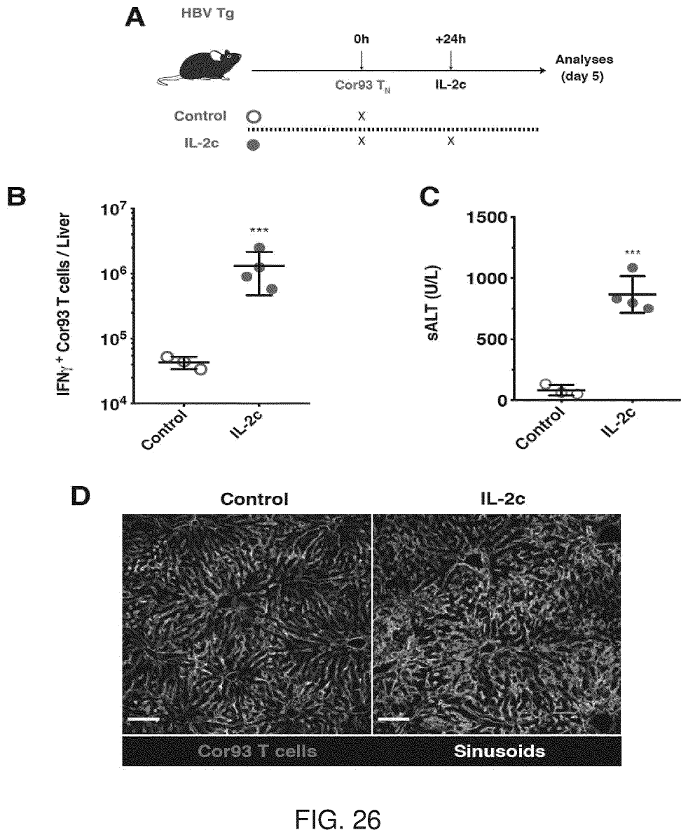

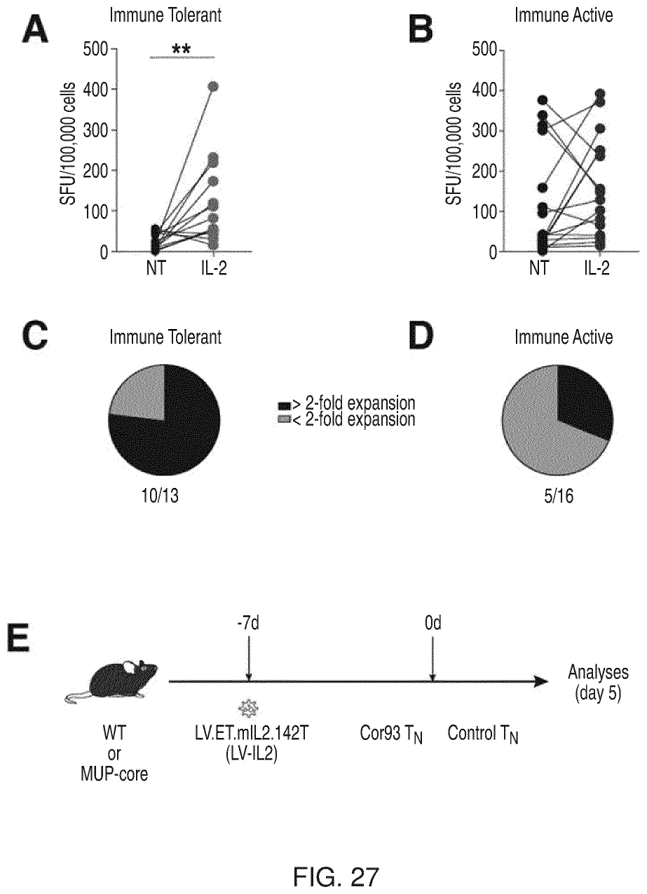

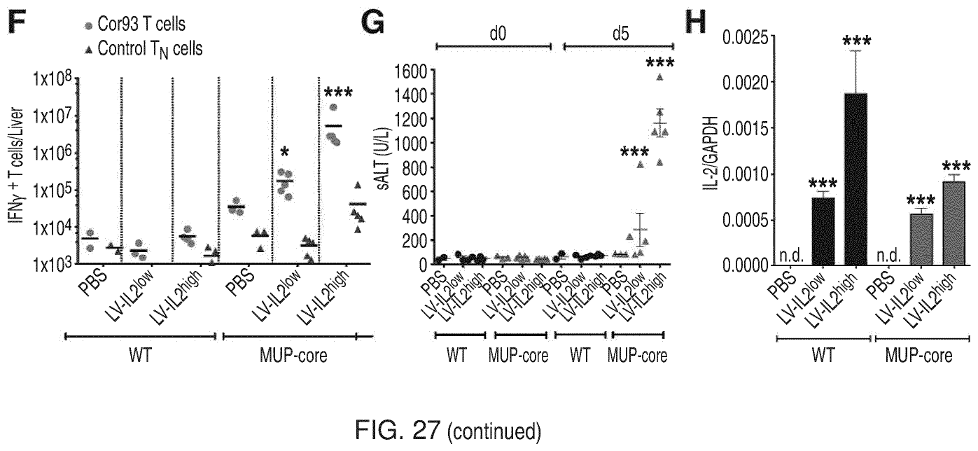

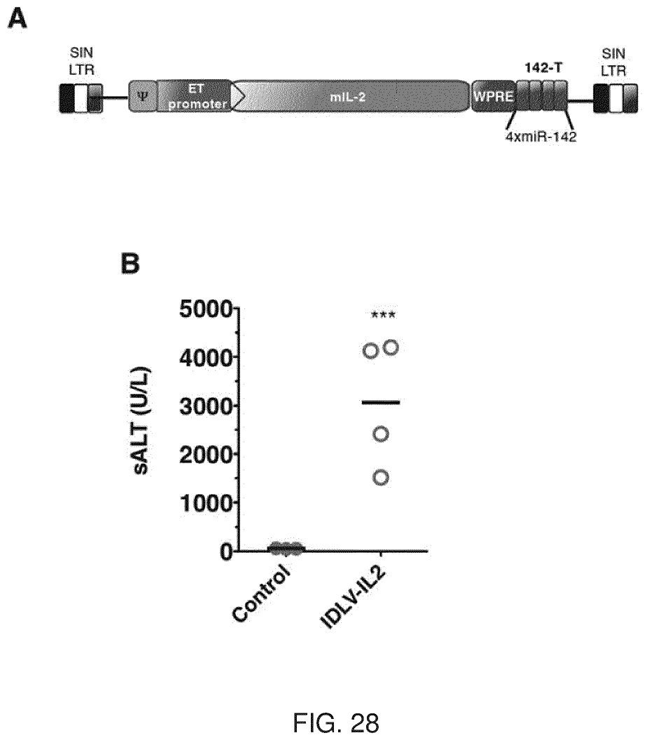

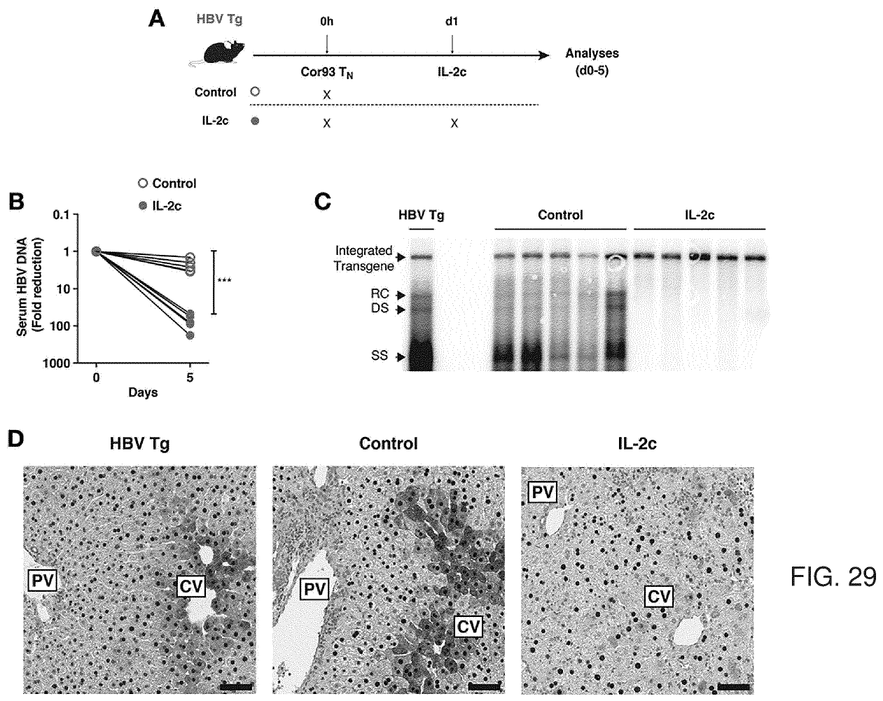

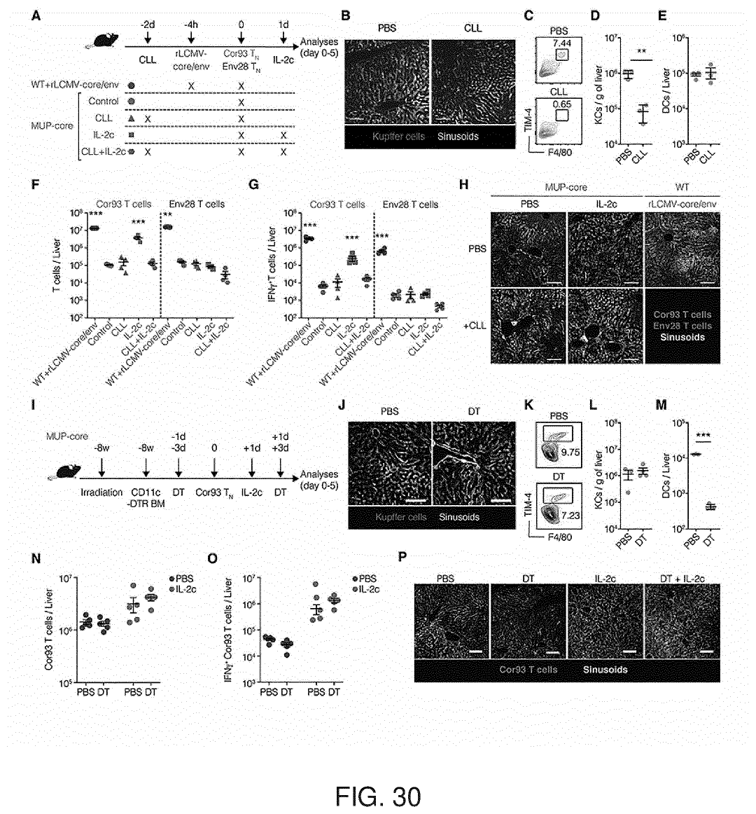

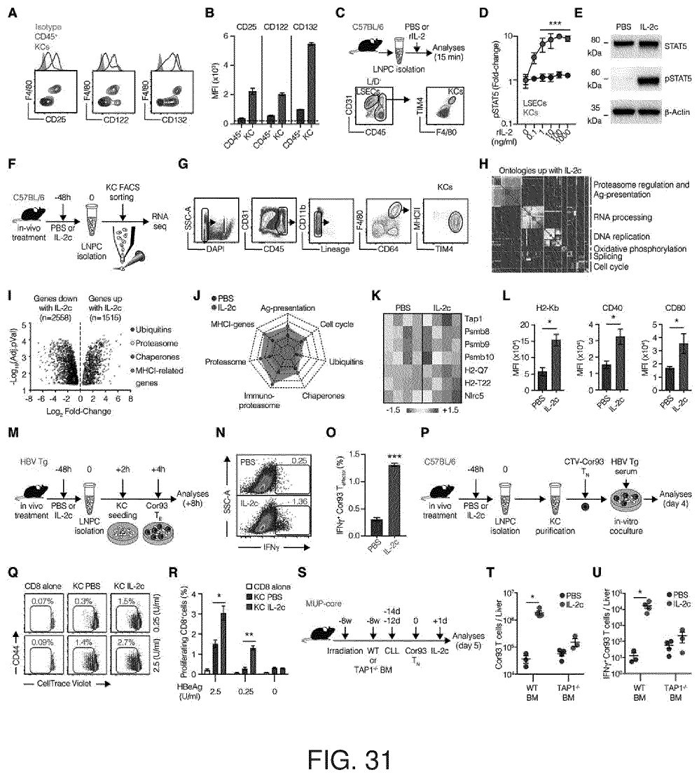

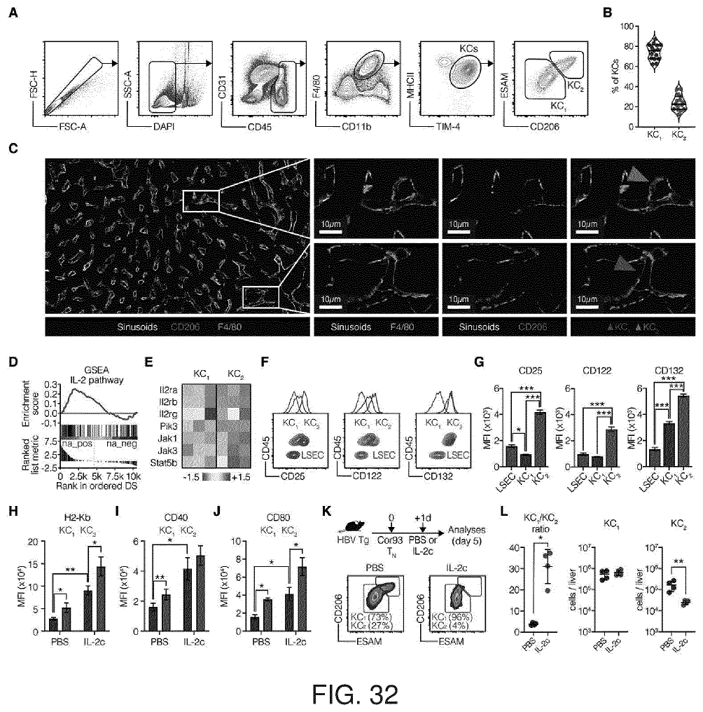

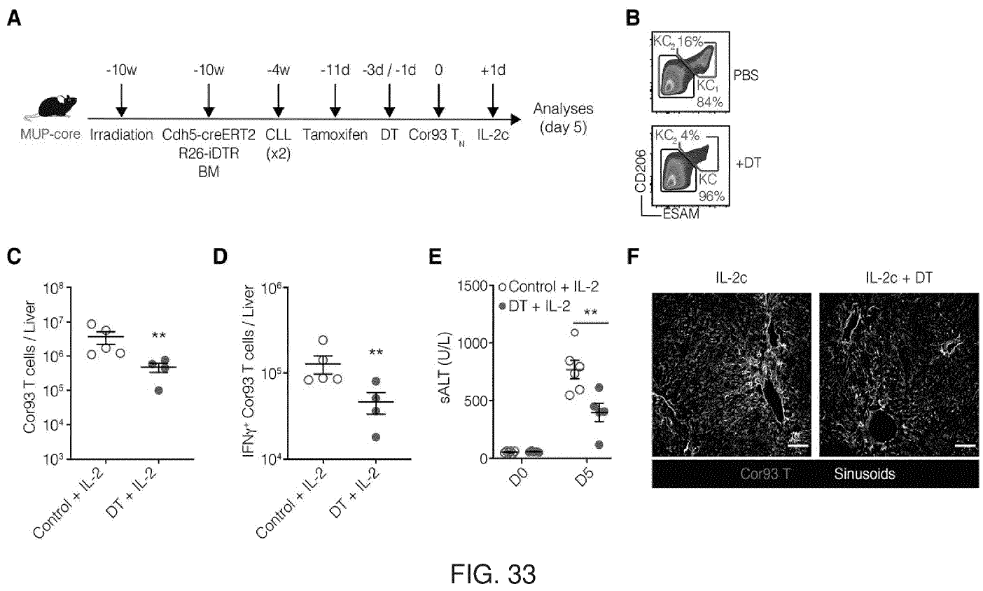

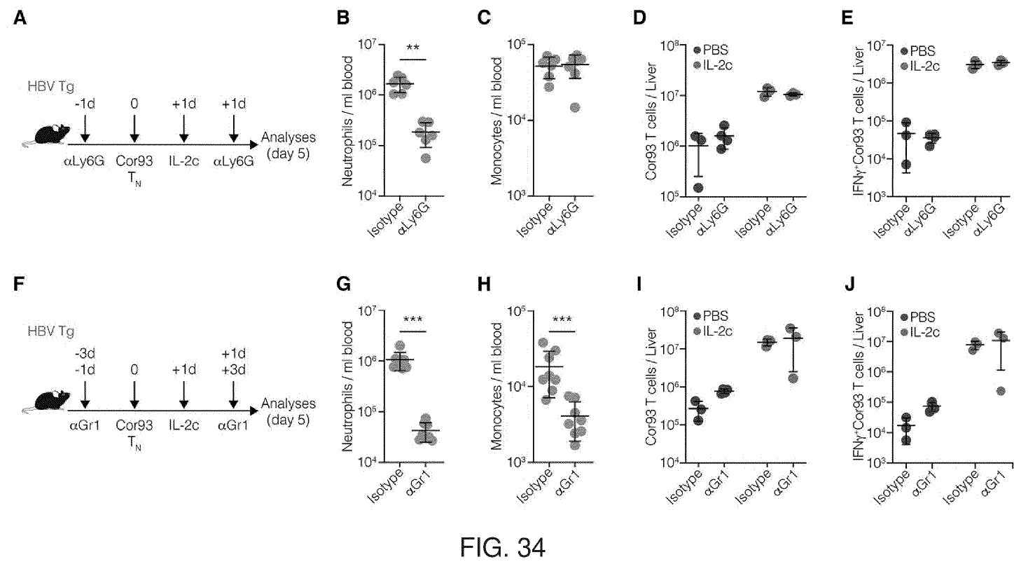

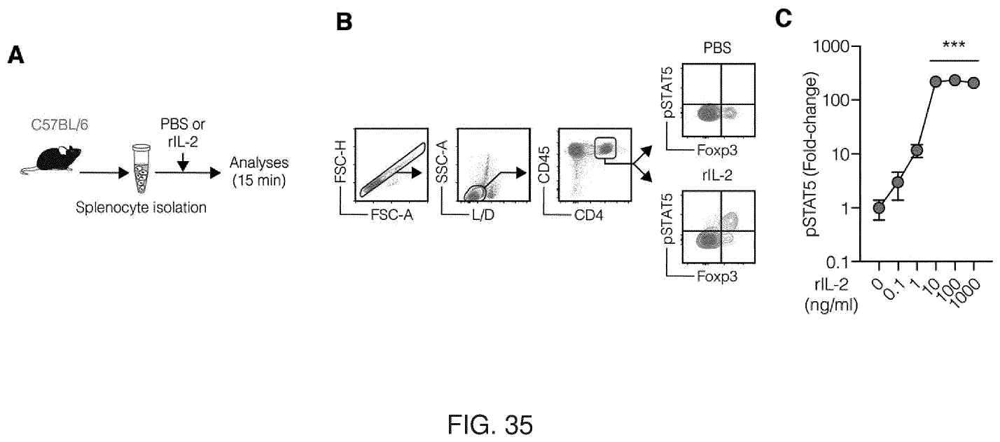

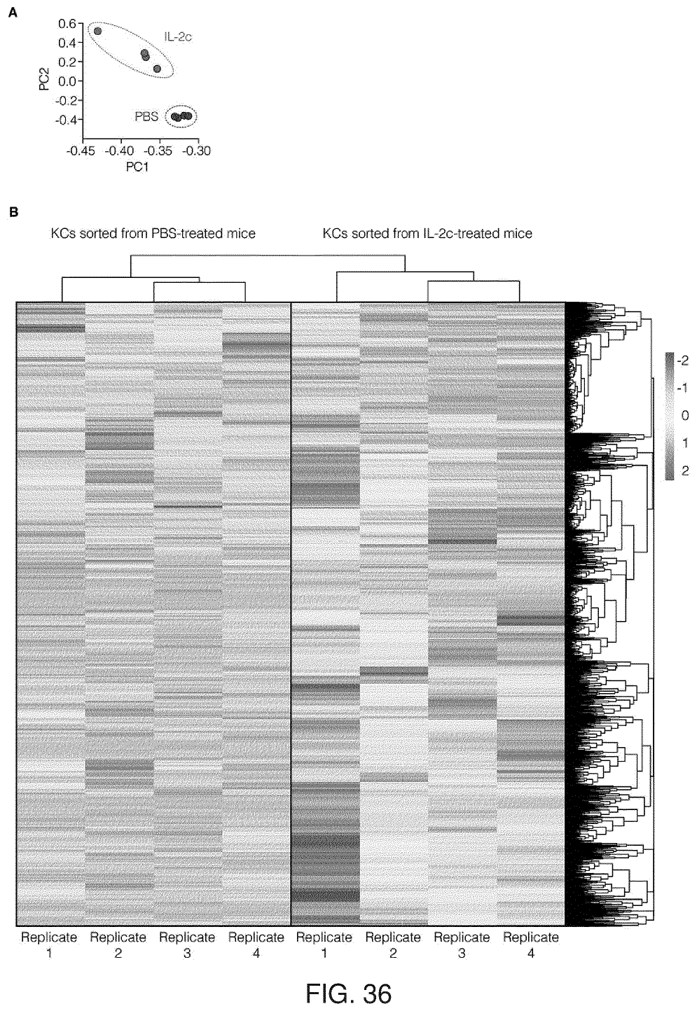

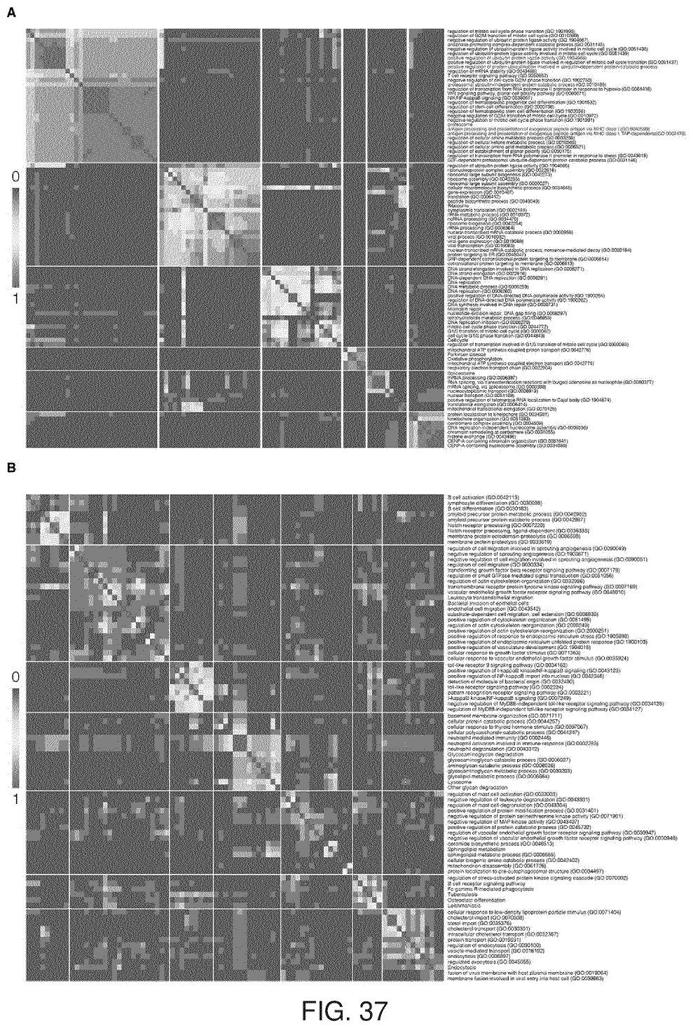

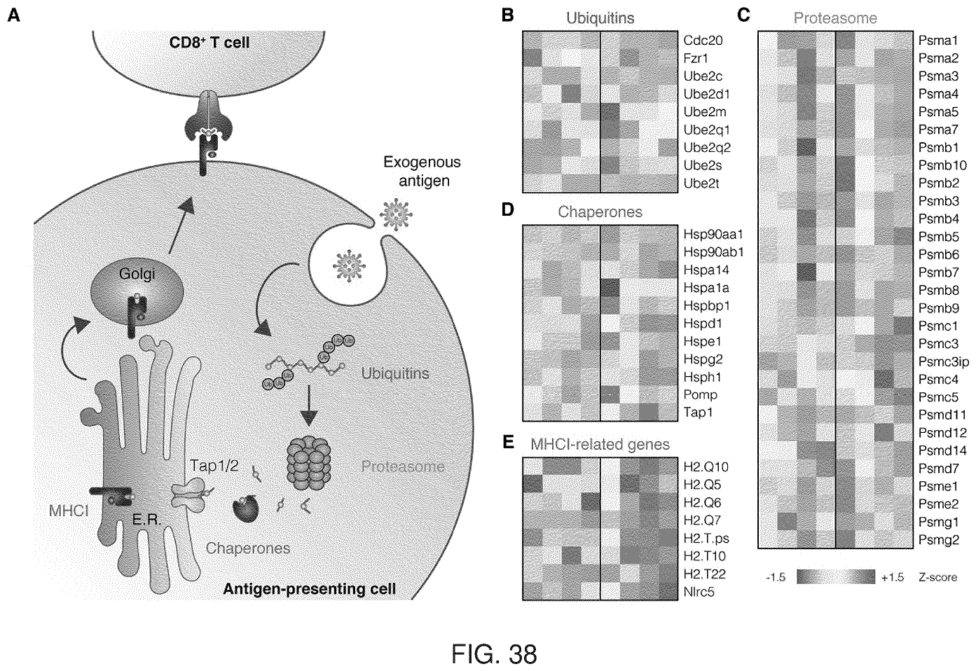

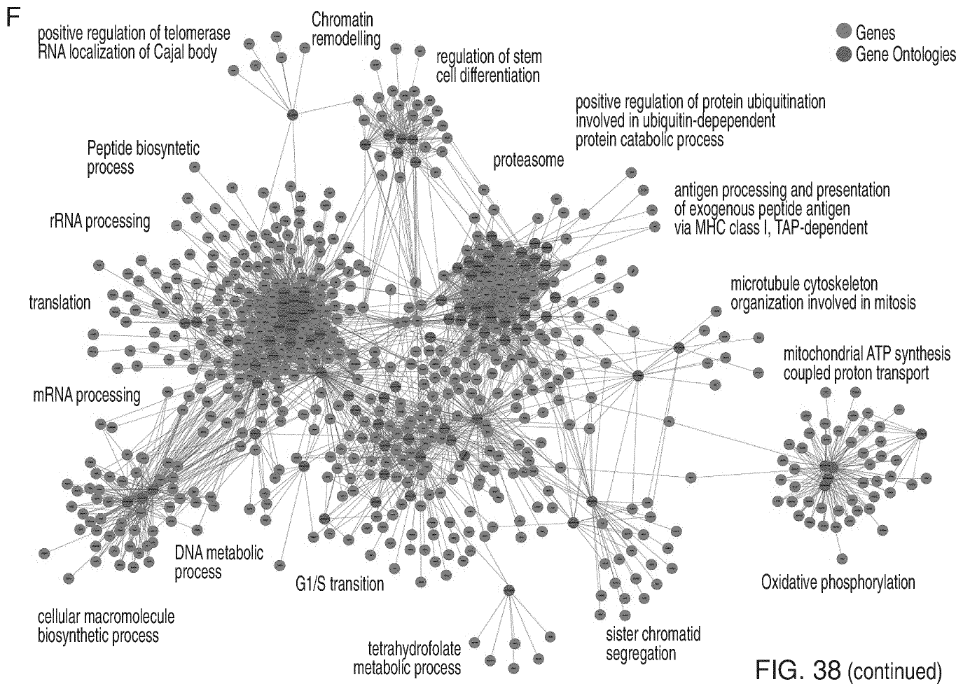

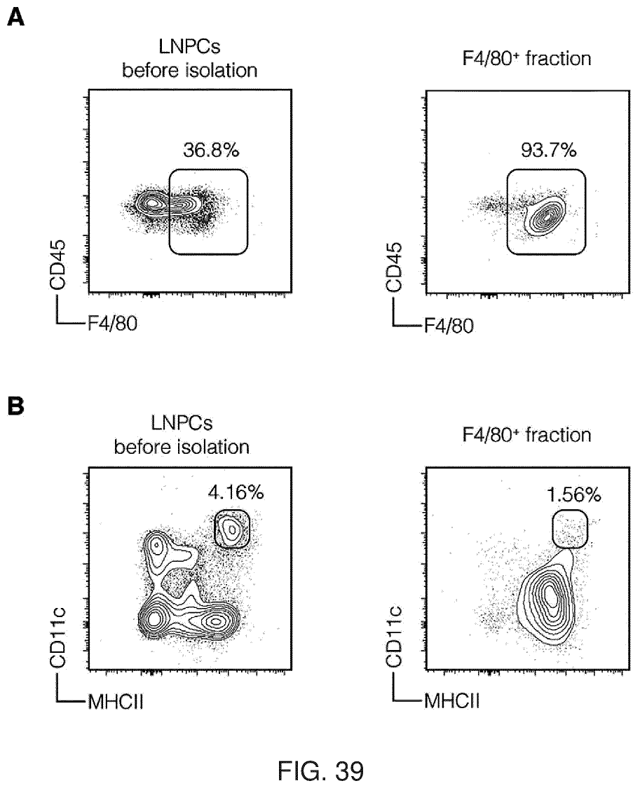

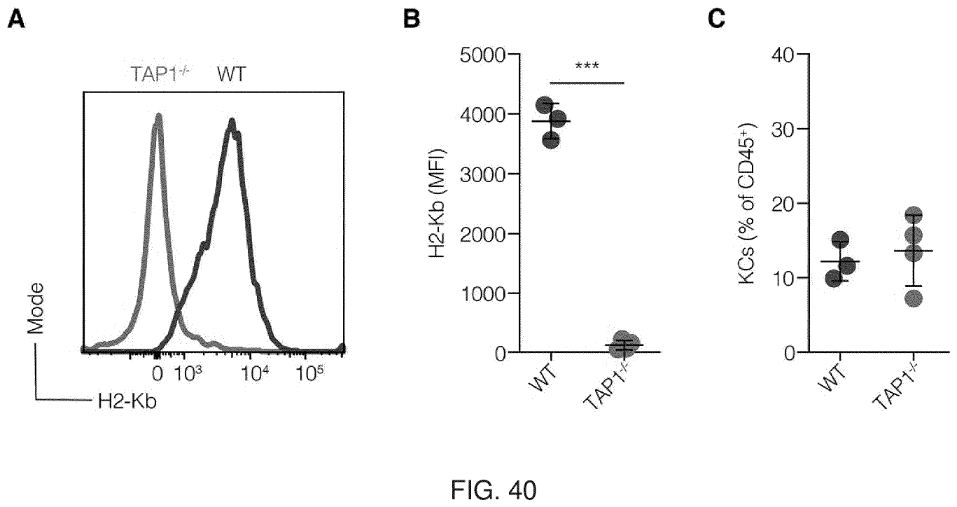

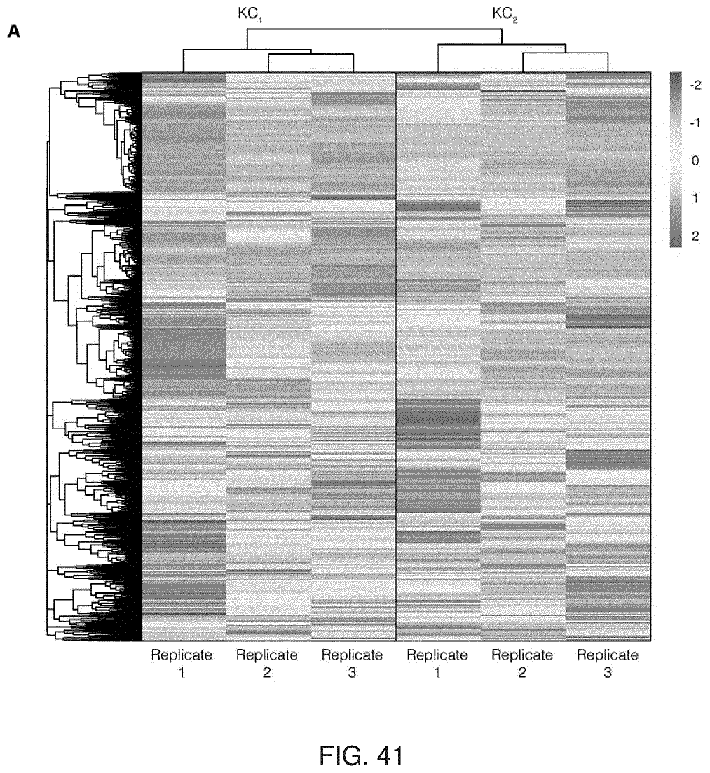

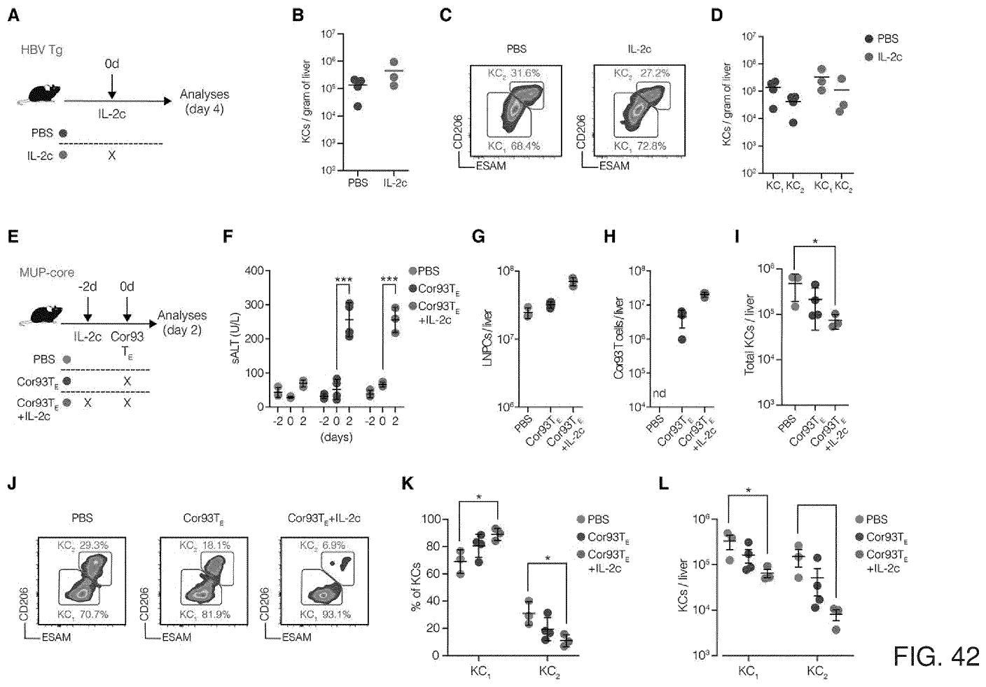

To shed light on the immune mechanisms underpinning early HBV unresponsiveness, we took advantage of dedicated mouse models to perform a detailed dynamic and molecular characterisation of HBV-specific CD8⁺ T cells undergoing priming in a non-inflamed liver expressing HBV antigens. In accordance with previous studies, envelope-specific naïve CD8⁺ TCR transgenic T cells (Env28 T_{N}) adoptively transferred into HBV replication-competent transgenic mice expressing all viral proteins in the hepatocyte (Guidotti, L. G., et al., 1995. J Virol 69, 6158-6169) (FIG. 1A) proliferated (FIG. 1B), but failed to develop IFN-γ-producing (FIG. 1C) or cytolytic capacities (FIG. 1D). As an effective CD8⁺ T cell response is readily induced in immunocompetent individuals exposed to HBV in adulthood, it remains to be determined whether this is due to cross-priming events in secondary lymphoid organs or whether the liver itself is capable of supporting full effector differentiation.

To begin addressing these issues, we setup a system whereby naïve CD8⁺ T cell priming is restricted to the liver. This was achieved by virtue of splenectomy followed by the injection of anti-CD62L blocking antibodies (FIG. 2A), so that subsequently injected naïve CD8⁺ T cells cannot home to peripheral lymph nodes (FIG. 3A-C). Naïve CD8⁺ TCR transgenic T cells specific for an epitope contained within the core protein of HBV (Cor93 T_{N}) were then injected into MUP-core transgenic mice (Guidotti, L. G., et al., 1994. J Virol 68, 5469-5475) (FIG. 2A) that exclusively express a non-secretable version of the HBV core protein in 100% of hepatocytes (FIG. 3D). Of note, the genetic incapacity of MUP-core mice to produce either core protein-containing virions or the secretable form of the HBV core protein (HBeAg)—both of which could be potentially up-taken and cross-presented by Kupffer cells (KCs) and/or hepatic dendritic cells (DCs)—further restricts Ag presentation to hepatocytes (Guidotti, L. G. et al., 2015. Cell 161, 486-500). Two additional groups of mice served as controls (FIG. 2A): i) wild-type (WT) mice; and ii) WT mice that are transduced with recombinant replication-defective, lymphocytic choriomeningitis virus (LCMV)-based vectors (Flatz, L. et al., 2010. Nat Med 16, 339-345) targeting a non-secretable version of the HBV core protein (rLCMV-core) to intrahepatic professional Ag-presenting cells (i.e. KCs and hepatic DCs) that are not naturally infected by HBV (FIG. 3D). That Ag recognition is restricted to either hepatocytes or KCs and hepatic DCs in MUP-core and rLCMV-transduced WT mice, respectively, was confirmed by experiments in which Cor93 T_{N} isolated 1 hour after transfer from liver, blood, lung and bone marrow showed surface up-regulation of CD69 (as a proxy for Ag recognition) only in the former organ (FIG. 3E). We then sought to characterise the fate and function of naïve CD8⁺ T cells undergoing intrahepatic priming. Consistent with previously studies and results shown in FIG. 1, HBV-specific naïve CD8⁺ T cells that recognised Ag in the liver underwent local activation (FIG. 3E), followed by proliferation, so that by day 3 after transfer we could recover ˜30-fold more intrahepatic Cor93 T cells in Ag-expressing mice than control animals (FIG. 2B). Whether T_{N} recognised Ag on hepatocytes or on KCs and hepatic DCs made no difference in their early expansion, as we recovered similar numbers of Cor93 T cells from the livers of MUP-core mice or from WT mice transduced with rLCMV-core (FIG. 2B). Ag recognition on these different cell types, however, made major differences in T cell differentiation and function. Whereas Ag recognition on KCs and hepatic DCs yielded bona fide effector cells endowed with IFN-γ-producing (FIG. 2C) and cytolytic abilities, Ag recognition on hepatocytes led to the generation of dysfunctional cells that produce little or no IFN-γ upon in vitro peptide re-stimulation (FIG. 2C), did not develop cytotoxic activity and instead up-regulated the inhibitory receptor PD-1 (FIG. 2D). Of note, the same dichotomous phenotype was observed independently of the number of adoptively transferred T_{N} (FIG. 4). Taken together, these results indicate that, depending upon the nature of the Ag-presenting cell, the liver can support the development of either functional or dysfunctional CD8⁺ T cells. Before delving deeper into the mechanisms underlying the immune dysfunction caused by hepatocellular Ag recognition, we performed a detailed kinetic analysis of the spatiotemporal determinants of naïve CD8⁺ T cells undergoing intrahepatic priming. We found that CD8⁺ T cells formed clusters beginning at day 3 after Ag recognition in the liver of both rLCMV-core transduced mice and MUP-core mice (FIG. 2E and FIG. 5A). In the former animals, T cell clusters were scattered throughout the liver lobule (FIG. 2E and FIG. 5A-B) in a pattern that is reminiscent to that observed during acute self-limited HBV infection in chimpanzees (note that access to liver biopsies in man is limited to chronic hepatitis cases). By contrast, CD8⁺ T cells formed clusters that coalesced around portal tracts in MUP-core mice (FIG. 2E and FIG. 5A-B), a situation that is reminiscent of chronic HBV infection in humans. Accumulation of Cor93 T cells in periportal clusters in MUP-core mice occurred in spite of the fact that the core protein is uniformly expressed in all hepatocytes (FIG. 3D) and that in the first few hours after transfer CD8⁺ T cells recognise Ag on hepatocytes that can be distant from portal tracts. Multiphoton intravital imaging of the liver revealed that clusters formed in WT mice transduced with rLCMV-core are dense, poorly perfusable and composed of largely immotile cells (FIG. 2E-F); by contrast clusters formed in MUP-core mice are looser, readily perfusable with intravascular dies and composed of more motile cells (FIG. 2E-F). The different T cell motility observed in the two settings is reminiscent of the interaction dynamics of T cell with lymph node-resident DCs during the initial phase of induction of immunity or tolerance, and with the general concept that long-lasting TCR engagements are required for the efficient priming of naïve T cells. By day 5-7, clusters in WT mice transduced with rLCMV-core start to disaggregate as cells move out from the liver into the peripheral blood, whereas clusters in MUP-core mice remain in place, possibly reflecting Ag persistence.

The notion that the liver is capable of supporting full effector differentiation is not without precedent but stands in contrast to the immunological dogma that T cell priming occurs exclusively in secondary lymphoid organs. Since CD4⁺ T cells are required for optimal priming of CD8⁺ T cells in secondary lymphoid organs under certain conditions, we asked whether T cell help was required for full effector differentiation upon hepatic priming in WT mice transduced with rLCMV-core. Antibody-mediated depletion of CD4⁺ T cells revealed that T cell help is not required for the expansion, differentiation and cluster formation of naïve CD8⁺ T cells transferred to rLCMV-core-transduced WT mice (FIG. 6A-E) and these results were confirmed in MHC-II-deficient hosts that genetically lack CD4⁺ T cells. As KCs and hepatic DCs—which are not natural targets of HBV—are both targeted by rLCMV, we next investigated whether the latter cell type is sufficient to efficiently support intrahepatic priming of naïve CD8⁺ T cells upon rLCMV injection. To this end, we depleted KCs (the major hepatic target of rLCMV) in WT mice through clodronate liposomes (CLL) injection (FIG. 7A-B). This treatment effectively depletes KCs while sparing hepatic DCs (FIG. 7B-D). As shown in FIG. 7E-G, KC depletion abolished T_{N} expansion and effector differentiation, indicating that hepatic DCs are dispensable for these processes in this experimental system. Whether KCs directly primed T_{N} or whether they functioned as Ag reservoirs handing over Ag to DCs (or other antigen-presenting cells) remains to be determined.

We next turned our attention to the immunological dysfunction induced in naïve CD8⁺ T cells by hepatocellular Ag presentation. Since MUP-core mice spontaneously develop neither Ag-specific CD8⁺ T cell responses nor liver pathology in face of undetectable neonatal/perinatal core protein expression, we reasoned that regulatory T cells (Tregs) might have contributed to the lack of effector differentiation characterising the transferred Cor93 T_{N}. To test this hypothesis, we depleted CD4⁺ T cells (including Tregs) from MUP-core mice prior to Cor93 T_{N} injection (FIG. 8A-B). As shown in FIG. 8C-E, CD4⁺ T cell depletion did not affect the expansion, fate and migratory pattern of Cor93 T_{N} transferred to MUP-core mice. To further confirm that hepatocellular Ag recognition by T_{N} leads to immunological dysfunction in a non-transgenic system and where the amount of antigen expression by hepatocytes can be modulated, we injected WT mice with a hepatotropic adeno-associated viral vector (AAV) that encodes a non-secretable version of the HBV core protein under the control of a hepatocyte-specific promoter (AAV-core³), using a dose of AAV that transduces ˜15-20% of hepatocytes (Guidotti, L. G. et al., 2015. Cell 161, 486-500) (FIG. 9A). Similar to what occurred in MUP-core mice, Ag recognition on AAV-core-transduced hepatocytes led to an initial Cor93 T cell expansion that was followed by a failure to express IFN-γ or developed cytolytic ability, and resulted in periportal cluster formation (FIG. 9B-D).

Finally, we investigated the fate of intrahepatic T_{N} primed by Ag presented by both hepatocytes as well as KCs and DCs. To this end we adoptively transferred WT and MUP-core mice with Cor93 and Env28 T_{N} prior to injecting them with PBS (control) or with rLCMV vectors encoding either the HBV envelope protein alone (rLCMV-env) or both the HBV core and envelope proteins (rLCMV-core/env). As expected, in WT mice T_{N} expanded and differentiated into IFN-γ-secreting cells only when cognate Ag is present (FIG. 2G). In MUP-core mice, injection of rLCMV-env allowed for Env28 (but not Cor93) T_{N} expansion and effector differentiation, indicating that i) innate immune signal carried by rLCMV vectors are not sufficient to overcome Cor93 T cell dysfunction and ii) dysfunctional Cor93 T cells do not produce soluble or membrane-bound mediators that inhibit Env28 T cell effector differentiation (provided that Ag is presented by KCs or hepatic DCs, FIG. 2G). Finally, injection of rLCMV-core/env to MUP-core mice led to Env28 (but not Cor93) T_{N} expansion and effector differentiation, indicating that, when Ag is presented by both hepatocytes as well as KCs and hepatic DCs, hepatocellular Ag presentation is dominant in inducing immune dysfunction (FIG. 2G).

Transcriptomic and Epigenomic Analyses of Cor93 CD8+ T Cells

In order to identify the molecular determinants of the immune dysfunction caused by hepatocellular Ag presentation and to identify potential targets for therapeutic intervention, we next performed transcriptomic and epigenomic analyses of Cor93 CD8⁺ T cells isolated from the livers of MUP-core mice and, as controls, of rLCMV-core-transduced WT mice. RNA sequencing (RNA-seq) analyses revealed broad transcriptional changes in intrahepatic Cor93 CD8⁺ T cells sorted from the two groups of mice at day 1, day 3 or day 7 after transfer (FIG. 10A, FIG. 11-12). A total of 1213 inducible genes were identified using stringent cut-offs (log FC>2.5, FDR<0.01 versus Cor93 T_{N}), of which 181 and 474 were expressed at higher levels in CD8⁺ T cells from WT mice injected with rLCMV-core (log FC>1.5, FDR<0.1) or from MUP-core mice (log FC<−1.5, FDR<0.1), respectively, in at least one time point (FIG. 10A, FIG. 12). Differences in gene expression between CD8⁺ T cells from WT mice injected with rLCMV-core or MUP-core mice were already evident at day 1 but became more pronounced at day 7 (FIG. 10A and FIG. 12), indicating progressive transcriptional divergence. Hepatic CD8⁺ T cells from rLCMV-core-transduced WT mice upregulated canonical genes of the T cell effector program such as Gzmk, Gzma, Gzmb, Ifng, Cxcr3, Ccr2, Ccr5, Cxcl10, S1pr5, Mx1, Mir155, KIrg1, Prdm1, Ctla2a, Ctla2b, Ly6a, Itgax, Itgad (FIG. 10A-B). By contrast, CD8⁺ T cells isolated from the livers of MUP-core mice at the same time points displayed a distinct gene expression program, characterised by selective expression of transcripts encoding for cytokines and chemokines (Ccl1, Csf2, Xcl1), growth factors and hormones (Areg, Calcb), inhibitory molecules (Pdcd1, Lag3, Havcr2) or surface markers (Siglect) and others (FIG. 10A-B, FIG. 12). These transcriptional differences were associated to distinct patterns of chromatin accessibility, measured by Assay for Transposase Accessible Chromatin (ATAC)-seq. Among all inducible ATAC-seq peaks (log FC>2.5, FDR<0.001 versus Cor93 T_{N}), CD8⁺ T cells from rLCMV-core-transduced WT mice or MUP-core mice displayed the most marked differences at days 3 and 7 after transfer (FIG. 10C, FIG. 13-14). Motif enrichment analysis on differentially accessible regions at day 7 revealed distinct networks of transcription factors (TFs) in cells from the two models (FIG. 10D). CD8⁺ T cells from WT mice injected with rLCMV-core displayed clear over-representation of binding sites for TFs controlling effector T cell differentiation, such as T-bet, RUNX and bHLH. On the other hand, CD8⁺ T cells from MUP-core mice were enriched in binding sites for AP-1, NFAT, EGR, NR4A and NF-κB TFs (FIG. 10D). Notably, enrichment of binding sites for some of these TFs, namely AP-1, NFAT and NR4A was previously associated to CD8⁺ T cell exhaustion. Our integrated genomic analyses indicated that Ag recognition on KCs and DCs can support priming and full differentiation into effector CD8⁺ T cells that are similar to those recovered from secondary lymphoid organs. By contrast, Ag recognition on hepatocytes initiates a defective differentiation program with progressive accumulation of chromatin and transcriptional landscape alterations that ultimately result in a dysregulated T cell phenotype. We next looked at the plasticity of the dysfunctional Cor93 T cells recovered from MUP-core livers. When Cor93 T cells were sorted from MUP-core livers 4 hours after injection and then transferred into WT mice previously injected with rLCMV-core, they were fully capable of expanding and differentiating into effector cells (FIG. 10E-G). By contrast, Cor93 T cells isolated from MUP-core livers at day 3 (a time point in which chromatin alterations are evident, FIG. 10B) were significantly impaired in their ability to expand and differentiate into IFN-γ-producing cells when transferred into WT mice previously injected with rLCMV-core (FIG. 10E-G). These data indicate that 3 days of hepatocellular Ag exposure are sufficient to render cells partially refractory to effector differentiation stimuli otherwise operative.

We then took a closer look at the differences in gene expression between the two models. Time-resolved Gene Set Enrichment Analysis (GSEA) identified distinct sets of over-represented gene ontology (GO) categories in the transcriptomes of CD8⁺ T cells from the two groups (FIG. 15A). CD8⁺ T cells isolated from the livers of rLCMV-core-transduced WT mice differentially expressed genes belonging to GO categories linked to the different phases of an effector immune response, including RNA transcription, mitochondrial respiration, cell proliferation, inflammatory response and adaptive immunity (FIG. 15A). CD8⁺ T cells from MUP-core livers failed to express genes linked to effector T cell responses beyond day 1, and instead expressed genes belonging to GO categories linked to tissue development and organ remodelling, cell differentiation and cell-matrix interaction (FIG. 15A). Notably, the transcriptional program of hepatic CD8⁺ T cells isolated from MUP-core mice at day 7 after transfer was not obviously overlapping with that of other known dysfunctional CD8⁺ T cell fates such as exhaustion or tolerance (FIG. 16-21). Indeed, genes with selective expression in CD8⁺ T cells from MUP-core livers were poorly expressed in reference transcriptomic datasets generated on splenic LCMV-specific exhausted CD8⁺ T cells (Scott-Browne, J. P. et al., 2016. Immunity 45, 1327-1340; and Pauken, K. E. et al., 2016. Science 354, aaf2807-1165) (FIG. 16-19) or on tolerant self-Ag-specific CD8⁺ T cells (Schietinger, A., et al., Science 335, 723-727) (FIG. 20-21).

Among the genes that are differentially expressed (FIG. 10A and FIG. 15B), we focused our attention on two known regulators of T cell function: Pdcd1 and 112. Pdcd1 was found to be hyper-expressed on Cor93 CD8⁺ T cells sorted from the livers of MUP-core mice (FIG. 10A and FIG. 15B), whereas 112 was found to be induced in the livers of rLCMV-core-transduced WT mice (FIG. 22) as well as hyper-expressed on Cor93 CD8⁺ T cells sorted from the livers of rLCMV-core-transduced WT mice (FIG. 10A and FIG. 15B).

Administration of IL-2 to Reinvigorate Intrahepatically-Primed, Dysfunctional CD8⁺ T cells

We assessed the functional consequences of these findings by treating MUP-core mice injected with Cor93 T_{N} with anti-PD-L1 blocking Abs, with recombinant IL-2 coupled with anti-IL-2 Abs (IL-2c) (Boyman, O., et al., 2006. Science 311, 1924-1927), or with a combination of both (FIG. 15C). IL-2c administration promoted both expansion and differentiation of Cor93 T cells into IFN-γ producing, cytotoxic effector cells (FIG. 15D-F), whereas anti-PD-L1 treatment either failed to do so when given alone or did not show a synergistic effect when given in combination with IL-2c (FIG. 15D-F). IL-2c administration 1 day after transfer of Cor93 T_{N} into MUP-core mice substantially rescued the transcriptional program of dysfunctional CD8⁺ T cells, as measured by RNA-seq at day 5 (FIG. 15 G-H, FIG. 22). Up to 58% of the genes with defective expression (hypo-expressed genes) in hepatic CD8⁺ T cells from MUP-core mice were upregulated in IL-2c-treated MUP-core mice, often reaching expression levels comparable to those detected in WT mice injected with rLCMV-core (FIG. 15 G-H, FIG. 23). Similarly, 55% of genes with abnormally high expression (hyper-expressed genes) in hepatic CD8⁺ T cells from MUP-core mice were downregulated by IL-2c treatment (FIG. 15G-H, FIG. 23). Genes rescued by IL-2c treatment in MUP-core mice included key components of the effector differentiation program such as Tbx21, Gmzk, Itgax, Itgad, Ccr5, Ctla2a, Ctla2b and others.

Since IL-2 administered at high doses has been shown to promote CD8⁺ T_{N} expansion and differentiation into effector-like cells even in the absence of Ag (Boyman, O., et al., 2006. Science 311, 1924-1927; and Kamimura, D. & Bevan, M. J., 2007. J Exp Med 204, 1803-1812), we next asked what the specificity of our treatment was. To this end, we co-transferred Ag-specific (Cor93) and irrelevant (Env28) T_{N} into MUP-core mice 24 hours prior to IL-2c treatment (FIG. 24A). Cor93 and Env28 T_{N} were also transferred into control WT mice previously injected with rLCMV vectors encoding for both HBV core and envelope proteins (rLCMV-core/env). To test whether IL-2c treatment had a direct effect on T_{N} or whether it required the presence of KCs, some animals were depleted of KCs by CLL injection prior to Cor93 T_{N} transfer (FIG. 24A). As shown in FIG. 24B-D, IL-2c improved the capacity of Ag-specific Cor93 T cells to expand, differentiate into IFN-γ-producing cells and accumulate in clusters scattered throughout the liver lobules, but it had no effect on irrelevant Env28 T_{N}. Interestingly, optimal in vivo reinvigoration of intrahepatically primed Cor93 T cells required the presence of KCs, as CLL-treated mice failed to improve T cell expansion and effector differentiation upon IL-2c treatment (FIG. 24B-D). We then reasoned that KCs might cross-present hepatocellular Ags and/or secrete some trophic factor for T cells. To distinguish between these possibilities, we generated MUP-core mice whose KCs lack Transporter associated with Antigen Processing 1 (TAP-1) and therefore cannot express MHC-I and present Ags to CD8⁺ T cells (FIG. 24E). As shown in FIG. 24F-G, these mice failed to respond to IL-2c, indicating that optimal reinvigoration of intrahepatically primed CD8⁺ T cells by IL-2 requires the capacity of KCs to cross-present hepatocellular Ags. Of note, baseline KC cross-presentation of hepatocellular Ags in this experimental system is negligible (Guidotti, L. G. et al., 2015. Cell 161, 486-500). Of note, whereas all KCs were found to express the beta and gamma subunits of the IL-2 receptor, we found that a subpopulation of KCs (representing ˜10% of the total KC population) also express the alpha subunit of the IL-2 receptor (CD25, FIG. 25).

To test the effect of IL-2 treatment in a more physiological setting, we decided to make use of HBV replication-competent transgenic mice that were neither splenectomised nor treated with anti-CD62L blocking Abs. As shown in FIG. 26, IL-2c administration to HBV replication-competent transgenic mice injected with Cor93 T_{N} promoted differentiation of Cor93 T cells into IFN-γ producing, cytotoxic effector cells that accumulated in clusters scattered throughout the liver lobules.

Therapeutic Potential of IL-2 Treatment for T Cell Reinvigoration During Chronic HBV Infection

We then hypothesised that HBV-specific T cells present in “immune tolerant” (IT) patients might have a different functional behaviour than those present in “immune active” (IA) patients and might be more closely related to T cells primed by hepatocytes in the mouse models described so far. We thus recruited 13 IT and 16 IA patients and tested the response of their HBV-specific T cells to IL-2. T cells from the different patients were stimulated with overlapping HBV peptides that cover the entire HBV proteome and cultured for 10 days in the presence or absence of recombinant human IL-2. The HBV peptide mixtures used were genotype-specific and matched according to the infecting HBV genotype. Subsequently, T cells where re-stimulated with the HBV peptide pools and the frequency of HBV-specific T cells was determined by IFN-γ ELISpot assay. From the IT patients, only very low frequencies of IFN-γ-secreting cells were detected when their HBV-specific T cells were expanded without the presence of IL-2 (mean=24 SFU/10⁵ cells, FIG. 27A). The addition of IL-2 during T cell expansion and during the ELISpot assay, however, could augment their frequency in 10 out of the 13 patients tested (mean=122 SFU/10⁵ cells; FIG. 27A, C). By contrast, HBV-specific T cells from IA patients did not require and could not be boosted with IL-2 during their expansion and their frequency was similar to that of IT patients in the presence of IL-2 (FIG. 27B, D). These data suggest that HBV-specific T cells from IT patients, but not from IA patients, resemble murine CD8⁺ T cells primed by hepatocytes in that they can expand and secrete IFN-γ only upon IL-2 treatment.

To test the clinical potential of IL-2 in a system that would limit its systemic toxicity (Spolski, R., et al., 2018. Nat Rev Immunol. doi:10.1038/s41577-018-0046-y), we generated third-generation, self-inactivating lentiviral vectors (LV.ET.mIL2.142T) that allow selective hepatocellular expression of murine IL-2 due to the presence of a synthetic hepatocyte-specific promoter/enhancer as well as specific microRNA 142 target sequences suppressing expression in hematopoietic cells (Brown, B. D., et al., 2006. Nat Med 12, 585-591) (FIG. 28). We injected WT or MUP-core mice with 2.5×10⁸ (LV-IL2^{low}) or 5×10⁸ (LV-IL2^{high}) transducing units (TU)/mouse, 7 days prior to Cor93 or control T_{N} injection (FIG. 27E). As shown in FIG. 27F-H, lentiviral-mediated hepatic expression of IL-2, even at a dose that transduces less than 10% of hepatocytes in vivo (data not shown), increased the capacity of Cor93 (but not control) T cells to expand and differentiate into IFN-γ-producing cells endowed with cytolytic capacities. A similar effect was observed when we used integrase mutant vectors (IDLV-IL2) that are completely defective for integrase-mediated integration and thus support only a transient wave of transgene expression (Lombardo, A. et al., 2007. Nat Biotechnol 25, 1298-1306; Mátrai, J. et al., 2011. Hepatology 53, 1696-1707) (FIG. 28B).

Treatment of HBV Infection

To test the effect of IL-2 treatment on HBV replication, we adoptively transferred Cor93-specific naïve CD8+ T cells into HBV replication-competent transgenic mice that were treated or not with IL-2c one day after T cell transfer. As shown in FIG. 29, IL-2 treatment resulted in a ˜100-fold reduction in serum HBV DNA. Moreover, Southern blot analysis on total hepatic DNA revealed undetectable HBV replicative DNA intermediates in all IL-2c treated animals (FIG. 29). Finally, immunohistochemical analyses showed undetectable cytoplasmic HBcAg upon IL-2 administration.

Together, these results reveal that the change in T cell effector function induced by IL-2 results in profound antiviral activity.

Discussion

We have delineated the spatiotemporal dynamics, molecular programs and functional consequences of naïve CD8⁺ T cells undergoing intrahepatic priming. Although some parameters that affect T cell differentiation (e.g. antigen levels, TCR affinity, the maturity of the developing immune system) were not systemically analysed in this study, we showed that hepatocellular presentation of HBV-derived Ags leads to a CD8⁺ T cell dysfunction that is markedly distinct from T cell alterations reported in other viral infections and cancer and, as such, is not readily responsive to anti-PD-L1 treatment. As immune checkpoint inhibitors are beginning to be tested in patients persistently infected with HBV, the results reported here should help interpreting the outcome of those studies and eventually inform the design of modified trials in selected cohorts of patients. Indeed, our data identified IL-2 as a key component of effective immunotherapeutic strategies against chronic HBV infection.

Materials and Methods

Mice

C57BL/6, CD45.1 (inbred C57BL/6), Balb/c, Thy1.1 (CBy.PL(B6)-Thy^{a}/ScrJ), β-actin-GFP [C57BL/6-Tg(CAG-EGFP)1Osb/J], β-actin-DsRed [B6.Cg-Tg(CAG-DsRed*MST)1Nagy/J], Tap1-deficient (B6.129S2-Tap1^{tm1Arp}/J), TCR-I [B6.Cg-Tg(TcraY1, TcrbY1)416Tev/J], CD11c-DTR [B6.FVB-1700016L2Rik^{Tg(tgax-DTR/EGFP)57Lan}/J] mice were purchased from Charles River or The Jackson Laboratory. MHC-II^{-/-} mice were obtained through the Swiss Immunological Mutant Mouse Repository (Zurich, Switzerland). MUP-core transgenic mice (lineage MUP-core 50 [MC50], inbred C57BL/6, H-2^{b}), that express the HBV core protein in 100% of the hepatocytes under the transcriptional control of the mouse major urinary protein (MUP) promoter, have been previously described (Guidotti, L. G., et al., 1994. J Virol 68, 5469-5475). HBV replication-competent transgenic mice (lineage 1.3.32, inbred C57BL/6, H-2^{b}), that express all of the HBV antigens and replicate HBV in the liver at high levels without any evidence of cytopathology, have been previously described (Guidotti, L. G., et al., 1995. J Virol 69, 6158-6169). In indicated experiments, MUP-core and HBV replication-competent transgenic mice were used as C57BL/6×Balb/c H-2^{bxd} F1 hybrids. Cor93 TCR transgenic mice (lineage BC10.3, inbred CD45.1), in which >98% of the splenic CD8⁺ T cells recognize a K^{b}-restricted epitope located between residues 93-100 in the HBV core protein (MGLKFRQL), have been previously described (Isogawa, M., et al., 2013. CPLoS Pathog 9, e1003490). Env28 TCR transgenic mice (lineage 6C2.36, inbred Thy1.1 Balb/c), in which ˜83% of the splenic CD8⁺ T cells recognize a L^{d}-restricted epitope located between residues 28-39 of HBsAg (IPQSLDSWWTSL), have been previously described (Isogawa, M., et al., 2013. CPLoS Pathog 9, e1003490). For imaging experiments Cor93 and TCR-I transgenic mice were bred against both β-actin-GFP and β-actin-DsRed mice, while Env28 transgenic mice were bred against β-actin-DsRed mice that were previously back-crossed more than 10 generations against Balb/c. Bone marrow (BM) chimaeras were generated by irradiation of MUP-core or C57BL/6 mice with one dose of 900 rad and reconstitution with the indicated BM; mice were allowed to reconstitute for at least 8 weeks before use. In some experiments, to achieve full reconstitution of Kupffer cells from donor-derived BM, mice were injected with 200 μl of clodronate-containing liposomes 28 and 31 days after BM injection. Mice were housed under specific pathogen-free conditions and used at 8-10 weeks of age. In all experiments, mice were matched for age, sex and (for the 1.3.32 animals) serum HBeAg levels before experimental manipulations. All experimental animal procedures were approved by the Institutional Animal Committee of the San Raffaele Scientific Institute.

Viruses and Viral Vectors

Replication-incompetent lymphocytic choriomeningitis virus (LCMV)-based vectors encoding for HBV core protein, HBV envelope protein, HBV core and envelope proteins, or Cre recombinase (termed rLCMV-core, rLCMV-env, rLCMV-core/env and rLCMV-cre, respectively) were generated, grown and titrated as described (Flatz, L. et al., 2010. Nat Med 16, 339-345). Mice were injected i.v. with 2.5×10⁵ infectious units of the indicated rLCMV vector 4 hours prior CD8⁺ T cells injection.

Adeno-associated viruses expressing GFP and HBV core protein (AAV-core-GFP) have already been described (Guidotti, L. G. et al., 2015. Cell 161, 486-500). Mice were injected with 3×10¹⁰ viral genomes (vg) of AAV-core-GFP 18 days prior to further experimental manipulation.

Third-generation, self-inactivating lentiviral vectors (LV.ET.mIL2.142T) that allow expression of murine IL-2 exclusively in hepatocytes due to the presence of a synthetic hepatocyte-specific promoter/enhancer as well as specific microRNA 142 target sequences that suppress expression in hematopoietic-lineage cells (Brown, B. D., et al., 2006. Nat Med 12, 585-591) were generated, produced and titrated as described (Cantore, A. et al., 2015. Sci Transl Med 7, 277ra28-277ra28). Briefly, the gene-synthesised murine interleukin 2 (mIL-2) cDNA was cloned into the previously described transfer vector pCCLsin.cPPT.ET.GFP.142T (Cantore, A. et al., 2015. Sci Transl Med 7, 277ra28-277ra28). Third-generation LVs were produced by calcium phosphate transient transfection of 293T cells of the transfer vector, the packaging plasmid pMDLg/p.RRE, pCMV.REV, the vesicular stomatitis virus glycoprotein G (VSV-G) envelope plasmid pMD2.G and the pAdvantage plasmid (Promega), as previously described (Cantore, A. et al., 2015. Sci Transl Med 7, 277ra28-277ra28). For integrase-defective lentiviral vector (IDLV) production, the pMDLg/p.RRE.D64Vint packaging with a mutant integrase was used instead of pMDLg/p.RRE, as described (Mátrai, J. et al., 2011. Hepatology 53, 1696-1707). Briefly, 9×10⁶ 293 T cells were seeded 24 hours before transfection in 15-cm dishes. Two hours before transfection culture medium was replaced with fresh medium. For each dish, a solution containing a mix of the selected transfer plasmid, the packaging plasmids pMDLg/pRRE and pCMV.REV, pMD2.G and the pAdvantage plasmid was prepared using 35, 12.5, 6.25, 9 and 15 μg of plasmid DNA, respectively. A 0.1×TE solution (10 mM Tris-HCl, 1 mM EDTA pH 8.0 in dH₂O) and water (1:2) was added to the DNA mix to 1,250 μL of final volume. The solution was left on a spinning wheel for 20-30 minutes, then 125 μl of 2.5M CaCl₂) were added. Immediately before transfection, a precipitate was formed by adding 1,250 μL of 2×HBS (281 mM NaCl, 100 mM HEPES, 1.5 mM Na₂HPO₄, pH 7.12) while the solution was kept in agitation on a vortex. The precipitate was immediately added to the culture medium and left on cells for 14-16 hours and after that the culture medium was changed. Supernatant was collected 30 hours after medium change and passed through a 0.22 μm filter (Millipore). Filtered supernatant was transferred into sterile 25×89 mm poliallomer tubes (Beckman) and centrifuged at 20,000 g for 120 min at 20° C. (Beckman Optima XL-100K Ultracentrifuge). Vector pellet was dissolved in the appropriate volume of PBS to allow a 500× concentration.

For LV titration, 10⁵ 293 T cells were transduced with serial vector dilutions in the presence of polybrene (16 μg/ml). Genomic DNA (gDNA) was extracted 14 days after transduction. gDNA was extracted by using Maxwell 16 Cell DNA Purification Kit (Promega) according to manufacturer's instructions. Vector copies per diploid genome (vector copy number, VCN) were quantified by quantitative PCR (qPCR) starting from 100 ng of template gDNA using primers (HIV sense: 5′-TACTGACGCTCTCGCACC-3′; HIV antisense: 5′-TCTCGACGCAGGACTCG-3′) and a probe (FAM 5′-ATCTCTCTCCTTCTAGCCTC-3′) designed to amplify the primer binding site region of LV. Endogenous DNA amount was quantified by a primers/probe set designed to amplify the human telomerase gene (Telo sense: 5′-GGCACACGTGGCTTTTCG-3′; Telo antisense: 5′-GGTGAACCTCGTAAGTTTATGCAA-3′; Telo probe: VIC 5′-TCAGGACGTCGAGTGGACACGGTG-3′ TAMRA). Copies per genome were calculated by the formula=[ng LV/ng endogenous DNA]×[n^{o} of LV integrations in the standard curve]. The standard curve was generated by using a CEM cell line stably carrying 4 vector integrants, which were previously determined by Southern blot and FISH analysis. All reactions were carried out in duplicate or triplicate in an ABI Prism 7900HT or Viia7 Real Time PCR thermal cycler (Applied Biosystems). Each qPCR run carried an internal control generated by using a CEM cell line stably carrying 1 vector integrant, which were previously determined by Southern blot and FISH analysis. Titer is expressed as transducing units_{293T} (TU)/mL and calculated using the formula TU/mL=[VCN×10⁵×1/dilution factor]. IDLV titer was determined on 293T cells 3 days after transduction using an ad hoc qPCR, which selectively amplifies the reverse transcribed vector genome (both integrated and non-integrated) discriminating it from plasmid carried over from the transient transfection (RT-LV; ΔU3 sense: 5′-TCACTCCCAACGAAGACAAGATC-3′, gag antisense: 5′ GAGTCCTGCGTCGAGAGAG-3′). Vector particles were measured by HIV-1 Gag p24 antigen immunocapture assay (Perkin Elmer) according to manufacturer's instructions. Vector infectivity was calculated as the ratio between titer and particles. Vector administration was carried out by tail vein injection in mice at 2.5-10×10⁸ TU/mouse, 7 days prior to T cell injection.

All infectious work was performed in designated BSL-2 or BSL-3 workspaces, in accordance with institutional guidelines.

Naïve T Cell Isolation, Adoptive Transfer and In Vivo Treatments

CD8⁺ T cells from the spleens of Cor93, Env28, TCR-I transgenic mice were purified by negative immunomagnetic sorting (Miltenyi Biotec). Mice were adoptively transferred with 2-5×10⁶, 2×10⁵ or 2×10⁴ CD8⁺ T cells. In selected experiments, mice were splenectomised and treated with 200 μg of anti-CD62L Ab (clone MEL-14, BioXcell) 48 hours and 4 hours prior to cell injection, respectively. Splenectomy was performed according to standard procedures (Reeves, J. P., et al., 2001. Curr Protoc Immunol Chapter 1, Unit 1.10). In selected experiments, CD4⁺ T cells were depleted by injecting i.v. 200 μg of anti-CD4 Ab (clone GK1.5, BioXcell) 3 days and 1 day prior to T cell transfer. In selected experiment, mice were treated with 200 μg of anti-PD-L1 (Clone 10F.9G2, BioXcell) 1 day before and 1 day and 3 days after T cell transfer. In selected experiments, WT or MUP-core mice were lethally irradiated and reconstituted with BM from CD11c-DTR mice; dendritic cells were subsequently depleted by injecting 25 ng/g of diphtheria toxin (Sigma) 3 days and 1 day prior to T cell transfer. In indicated experiments, Kupffer cells were depleted by intravenous injection of clodronate-containing liposomes 2 days prior to T cell injection, as described (Sitia, G. et al., 2011. PLoS Pathog 7, e1002061). IL-2/anti-IL-2 complexes (IL-2c) were prepared by mixing 1.5 μg of rIL-2 (BioLegend) with 50 μg anti-IL-2 mAb (clone S4B6-1, BioXcell) per mouse, as previously described (Boyman, O., et al., 2006. Science 311, 1924-1927). Mice were injected with IL-2c i.p. one after T cell transfer.

Cell Isolation and Flow Cytometry

Single-cell suspensions of livers, spleens, and lymph nodes were generated as described (Iannacone, M. et al., 2005. Nat Med 11, 1167-1169; and Tonti, E. et al., 2013. Cell Reports 5, 323-330). Kupffer cell isolation was performed as described (Guidotti, L. G. et al., 2015. Cell 161, 486-500). All flow cytometry stainings of surface-expressed and intracellular molecules were performed as described (Guidotti, L. G. et al., 2015. Cell 161, 486-500). Cell viability was assessed by staining with Viobility™ 405/520 fixable dye (Miltenyi). Abs used included: anti-CD3 (clone: 145-2C11, Cat #562286, BD Biosciences), anti-CD11 b (clone: M1/70, Cat #101239), anti-CD19 (clone: 1D3, Cat #562291 BD Biosciences), anti-CD25 (clone: PC61, Cat #102015), anti-CD31 (clone: 390, Cat #102427), anti-CD45 (clone: 30-F11, Cat #564279 BD Biosciences), anti-CD49b (clone: DX5, Cat #562453 BD Biosciences), anti-CD64 (clone: X54-5/7.1, Cat #139311), anti-F4/80 (clone: BM8, Cat #123117), anti-Ly6G (clone: 1A8, Cat #562700 BD Biosciences), anti-I-A/I-E (clone: M5/114.15.2, Cat #107622), anti-TIM4 (polyclonal, Cat #orb103599 Biorbyt), anti-CD69 (clone: H1.2F3, Cat #104517), anti-CD45.1 (clone: A20, Cat #110716), anti-IFN-γ (clone: XMG1.2, Cat #557735 BD Biosciences), anti-CD4 (clone: RM4-5, Cat #553048 BD Biosciences), anti-CD11c (clone: N418, Cat #117308), anti-1-Ab (clone: AF6-120.1, Cat #116420), anti-PD-1 (clone: J43, Cat #17-9985 eBioscience). All Abs were purchased from BioLegend, unless otherwise indicated. Recombinant dimeric H-2L^{d}:Ig and H-2K^{b}:Ig fusion proteins (BD Biosciences) complexed with peptides derived from HBsAg (Env28-39) or from HBcAg (Cor93-100), respectively, were prepared according to the manufacturer's instructions. Dimer staining was performed as described (Iannacone, M. et al., 2005. Nat Med 11, 1167-1169). All flow cytometry analyses were performed in FACS buffer containing PBS with 2 mM EDTA and 2% FBS on a FACS CANTO or LSRII (BD Biosciences) and analyzed with FlowJo software (Treestar).

Cell Sorting

Single-cell suspensions from spleens and livers were stained with Viobility 405/520 fixable dye (Miltenyi), with PB-conjugated anti-CD8a (clone 53-6.7) and PE-conjugated anti-CD45.1 Abs. Live CD8⁺ CD45.1⁺ cells were sorted on a MoFlo Legacy (Beckman Coulter) cell sorter in a buffer containing PBS with 2% FBS. Cells were always at least 98% pure.

RNA Sequencing Analyses—RNA Purification and Library Preparation

Total RNA was purified from 8,000-300,000 sorted cells by using ReliaPrep RNA Cell Miniprep System (Promega). Sequencing libraries were generated using the Smart-seq2 method (Picelli, S. et al., 2014 Nat Protoc 9, 171-181). Briefly, 5 ng of RNA was retrotranscribed and cDNA was amplified using 15 cycles and purified with AMPure XP beads (Beckman Coulter). After purification, the concentration was determined using Qubit 3.0 (Life Technologies) and the size distribution was assessed using Agilent 4200 TapeStation system. Then, the tagmentation reaction was performed starting from 0.5 ng of cDNA for 30 minutes at 55° C. and the enrichment PCR was carried out using 12 cycles. Libraries were then purified with AMPure XP beads, quantified using Qubit 3.0 and single-end sequenced (75 bp) on an Illumina NextSeq 500.

RNA Sequencing Analyses—Data Processing and Analysis

Reads were generated on NextSeq 500 (IIlumina) instrument following manufacturer's recommendations. Single end reads (75 bp) were aligned to the mm10 reference genome using STAR (Dobin, A. et al., 2013 Bioinformatics 29, 15-21) aligner. featureCounts function from Rsubread package (Liao, Y., et al., 2013. Nucleic Acids Res 41, e108-e108) was used to compute reads over RefSeq Mus musculus transcriptome, with option minMQS set to 255. Further analyses were performed with edgeR R package (Robinson, M. D., et al., 2010. Bioinformatics 26, 139-140). Genes with an RPKM (Reads per kilo base per million) value higher than 1 in at least two samples were retained. Pearson's correlation was computed for each couple of samples on log transformed RPKM. Principal component analysis was performed on CPM (Counts per million) values adjusted for batch covariate with ComBat function from sva R package (Leek, J. T., et al., 2012. Bioinformatics 28, 882-883). Read counts were normalised with the Trimmed Mean of M-values (TMM) method (Robinson, M. D. & Oshlack, A., 2010. Genome Biol 11, R25) using calcNormFactors function and dispersion was estimated with the estimateDisp function. Differential expression across different conditions was evaluated fitting a negative binomial generalised linear model on the dataset with glmQLFit function and then performing a quasi-likelihood (QL) F-test with glmQLFTest function. Batch information was included in the design as covariate.

RNA Sequencing Analyses—Definition of Induced and Differentially Expressed Genes

We first defined inducible genes, namely those genes with log 2FC>2.5 and FDR<0.01 relative to naïve T cells in at least one condition or time point. For each comparison, only genes with an RPKM value higher than 1 in at least two samples in the comparison were selected. For each time point, induced genes were classified as expressed at higher levels in the WT+rLCMV-core condition setting FDR<0.1 and log 2FC>1.5 (WT+rLCMV-core vs MUP-core) as cut-offs. Genes with a FDR<0.1 and a log 2FC<−1.5 in the WT+rLCMV-core vs MUP-core comparison were classified as expressed at higher levels in MUP-core. The remaining genes were defined as non-differentially expressed between WT+rLCMV-core and MUP-core.

RNA Sequencing Analyses—Gene Ontology (GO) Analyses

For each time point Gene set enrichment analysis (GSEA) (Subramanian, A. et al., 2005. Proc Natl Acad Sci USA 102, 15545-15550) was performed on a pre-ranked list of genes based on log 2FC values for WT+rLCMV-core versus MUP-core comparison. GSEA analysis was performed with clusterProfiler R package (Yu, G., et al., 2012. OMICS 16, 284-287) on Biological Processes ontology from org.Mm.eg.db database. For each time point gene sets with a q-value <0.1 were selected. For representation purposes all significant gene sets were summarised using REVIGO (Supek, F., et al., 2011. PLoS ONE 6, e21800) setting similarity at 0.7.

RNA Sequencing Analyses—Expression of DEGs in Published Datasets

RNA-seq/SRA data were downloaded from the Gene Expression Omnibus repository (GEO: https://www.ncbi.nlm.nih.gov/geo/) and converted to the FastQ format. Reads were then aligned against the whole Mus musculus mm10 genome build using STAR aligner (v 2.6.0a) with default options, generating BAM files. Read counts for all expressed genes (Ensembl annotation v94; GENCODE M19) were obtained using featureCounts (Rsubread v 3.7). Features with <1 counts per million (cpm) were filtered out. The resulting count matrix was then normalised using the normalisation factors generated by the upperquartile method (Bullard, J. H., et al., 2010. BMC Bioinformatics 11, 94) implemented in edgeR Bioconductor package. Hierarchical cluster analysis was performed on rpkm (reads per kilobase per million of mapped reads) values. The similarity of the samples was measured using the Pearson correlation coefficient and the complete-linkage was used as the distance measure of the agglomerative hierarchical clustering.

Illumina BeadChip Data Analysis.

The normalised expression matrix was downloaded from the Gene Expression Omnibus (GEO) repository. Genes whose expression level corresponded to the 65th percentile of the distribution of the log 2 expression values were considered to be expressed.

RNA Sequencing Analyses—Definition of Rescued, Partially Rescued and Non-Rescued Genes by IL-2c

Only genes with an RPKM value higher than 1 in at least one sample were retained. Read counts were normalised with the Trimmed Mean of M-values (TMM) method using calcNormFactors function. Differential expression across different conditions was evaluated computing log₂FCs on CPM values. Inducible genes were defined first, namely those genes with log₂FC>2.5 in WT+rLCMV-core versus naïve T cells or MUP-core versus naïve T cells comparisons. For both comparisons, only genes with an RPKM value higher than 1 in at least one sample in the comparison were selected. Induced genes were classified as expressed at higher levels in the WT+rLCMV-core condition setting log₂FC>1.5 (WT+rLCMV-core vs MUP-core) as cut-off. Genes with a log₂FC<−1.5 in the WT+rLCMV-core vs MUP-core comparison were classified as expressed at higher levels in MUP-core. We then classified WT+rLCMV-core >MUP-core genes as rescued if their log₂FC in IL-2c treated MUP-core versus MUP-core comparison was higher than 1.5, partially rescued if their log₂FC in IL-2c treated MUP-core versus MUP-core comparison was between 1.5 and 1 and not rescued if their log₂FC in IL-2c treated MUP-core versus MUP-core comparison was lower than 1. Conversely MUP-core >WT+rLCMV-core genes were defined as rescued if their log₂FC in IL-2c treated MUP-core versus MUP-core comparison was lower then −1.5, partially rescued if their log₂FC in IL-2c treated MUP-core versus MUP-core comparison was between −1.5 and −1 and not rescued if their log₂FC in IL-2c treated MUP-core versus MUP-core comparison was higher then −1.

ATAC-seq

ATAC (Assay for Transposase Accessible Chromatin)-seq was performed as described (Buenrostro, J. D., et al., 2015. Curr Protoc Mol Biol 109, 21.29.1-9) with slight modifications. Briefly, 8,000-50,000 cells per replicate were sorted and centrifuged at 1,600 rpm for 5 minutes. Then, the transposition reaction was performed using digitonin 1% (Promega), Tn5 transposase and TD Buffer (Illumina) for 45 minutes at 37° C. Immediately following transposition, the reaction was stopped using a solution of 900 mM NaCl and 300 mM EDTA, 5% SDS and Proteinase K (Sigma-Aldrich) for 30 minutes at 40° C. Transposed DNA fragments were purified using AMPure XP beads (Beckman Coulter), barcoded with dual indexes (Illumina Nextera) and PCR amplified with KAPA HiFi PCR Kit (KAPA Biosystems). Then, the concentration of the library was determined using Qubit 3.0 (Life Technologies) and the size distribution was assessed using Agilent 4200 TapeStation system. Libraries were single-end sequenced (75 bp) on an Illumina NextSeq 500.

ATAC-seq—Data Processing

Reads were generated on NextSeq 500 (Illumina) instrument following the manufacturer's recommendations. Single end reads (75 bp) were aligned to the mm10 reference genome using BWA (Li, H. & Durbin, R., 2009. Bioinformatics 25, 1754-1760) aligner. Bam files were processed using samtools (Li, H. et al., 2009. Bioinformatics 25, 2078-2079) and BEDTools (Quinlan, A. R. & Hall, I. M., 2010. Bioinformatics 26, 841-842) suites: reads with a mapping quality lower then 15 or duplicated were discarded. Moreover, unassigned reads and reads mapped on chromosomes Y and M were removed. MACS2 (Zhang, Y. et al., 2009. Genome Biol 9, R137) callpeak function with parameters -g mm --nomodel --shift -100--extsize 200 was used for peak calling. For each sample peaks with a q-value lower then 1e-10 were selected. Passing filter peaks from all samples were then merged with mergeBed function form BEDTools, resulting in 72884 regions. Reads counts were computed on this set of regions using coverageBed function form BEDTools.

ATAC-seq—Definition of Induced and Differentially Induced ATAC-seq Peaks

The set of 72884 regions was annotated using ChlPpeakAnno R package (Zhu, L. J. et al., 2010 BMC Bioinformatics 11, 237). Each region was associated to the gene with the closest TSS.

Further analyses were performed with edgeR R package. Pearson's correlation was computed for each couple of samples on log transformed CPM. As previously described for RNA-seq data, read counts were normalised with the TMM method using calcNormFactors function and dispersion was estimated with the estimateDisp function. Differences in peaks intensities across different conditions were evaluated fitting a negative binomial generalised linear model on the dataset with glmQLFit function and then performing a quasi-likelihood (QL) F-test with glmQLFTest function. Batch information was included in the design as covariate.

Inducible peaks were defined first, namely those regions with log₂FC>2.5 and FDR<0.001 relative to naïve T cells in at least one condition or time point. For each time point, induced peaks were classified as induced at higher levels in the WT+rLCMV-core condition setting FDR<0.1 and log₂FC>1.5 (WT+rLCMV-core vs MUP-core) as cut-offs. Peaks with a FDR<0.1 and a log₂FC<−1.5 in the WT+rLCMV-core vs MUP-core comparison were classified as induced at higher levels in MUP-core. The remaining peaks were defined as non-differentially induced between WT+rLCMV-core and MUP-core.

Motif enrichment analysis was then performed with HOMER (Heinz, S. et al., 2010. Molecular Cell 38, 576-589) using findMotifsGenome.pl script. For each time point we ranked ATAC-seq peaks on log₂FCs in WT+rLCMV-core versus MUP-core comparison. We selected the top 200 regions with higher and lower log₂FC values. These sets of regions were compared to a background composed by a set of 3899 regions with unchanged intensities (FDR>0.1 and abs(log₂FC)<0.5) between both MUP-core and WT+rLCMV-core versus naïve in all time points.

RT-qPCR

Total RNA was extracted from frozen livers using ReliaPrep™ RNA Tissue Miniprep System (Promega), according to the manufacturer's instructions, as described (Fioravanti, J. et al., 2017. J Hepatol 67, 543-548), genomic DNA contamination was removed using Ambion® TURBO DNA-Free™ DNase. 1 μg of total RNA was reverse transcribed with Superscript IV Vilo (Life Technologies) prior to qPCR analysis for mouse 112 (TaqMan Mm00434256, Life Technologies), ifng (TaqMan Mm01168134, Life Technologies), HBV core (forward TACCGCCTCAGCTCTGTATC, reverse CTTCCAAATTAACACCCACCC, probe TCACCTCACCATACTGCACTCAGGCAA). Reactions were run and analysed on ViiA7 instrument (Life Technologies). All experiments were performed in triplicate and normalised to the reference gene GAPDH.

Confocal Immunofluorescence Histology and Histochemistry

Confocal microscopy analysis of livers was performed as described (Guidotti, L. G. et al., 2015. Cell 161, 486-500). The following primary Abs were used for staining: anti-F4/80 (BM8, Invitrogen), anti-cytokeratin 7 (EPR17078, Abcam), anti-Lyve-1 (NB600-1008, Novus Biological), anti-activated caspase 3 (AF835, R&D Systems), anti-Ki67 (TEC-3, DAKO), anti-HBcAg (polyclonal, Dako). The following secondary Abs were used for staining: Alexa Fluor 488-, Alexa Fluor 514-, Alexa Fluor 568-, or Alexa Fluor 647-conjugated anti-rabbit or anti-rat IgG (Life Technologies). Images were acquired on an inverted Leica microscope (TCS STED CW SP8, Leica Microsystems) with a motorised stage for tiled imaging. To minimise fluorophore spectral spillover, we used the Leica sequential laser excitation and detection modality. The bleed-through among sequential fluorophore emission was removed applying simple compensation correction algorithms to the acquired images. The semiautomatic surface-rendering module in Imaris (Bitplane) was used to create 3D volumetric surface objects corresponding either to individual cells or to the liver vascular system. Signal thresholds were determined using the Imaris Surface Creation module, which provides automatic threshold. T cells were tracked manually for single cell distance from the center of each bile duct (CK7⁺) using Fiji.

For H&E and HBcAg immunohistochemistry, livers were perfused with PBS, harvested in Zn-formalin and transferred into 70% ethanol 24 hours later. Tissue was then processed, embedded in paraffin and stained as previously described (Guidotti, L. G. et al., 2015. Cell 161, 486-500). Bright-field images were acquired through an Aperio Scanscope System CS2 microscope and an ImageScope program (Leica Biosystem) following the manufacturer's instructions.

Intravital Multiphoton Microscopy

Liver intravital multiphoton microscopy was performed as described (Guidotti, L. G. et al., 2015. Cell 161, 486-500; and Benechet, A. P., et al., 2017. Methods Mol. Biol. 1514, 49-61). Liver sinusoids were visualised by injecting nontargeted Quantum Dots 655 (Invitrogen) i.v. during image acquisition. Images were acquired with a LaVision BioTec TriMScope II coupled to a Nikon Ti-U inverted microscope enclosed in a custom-built environmental chamber (Life Imaging Services) that was maintained at 37-38° C. with heated air. Continuous body temperature monitoring through a rectal probe was performed to ensure that a narrow range of 37-38° C. was maintained at all times. Fluorescence excitation was provided by two tunable femtosecond (fs)-pulsed Ti:Sa lasers (680-1080 nm, 120 fs pulse-width, 80 MHz repetition rate, Ultra II, Coherent), an Optical Parametric Oscillator (1000-1600 nm, 200 fs pulse-width, 80 MHz repetition rate, Chameleon Compact OPO, Coherent). The setup includes four non-descanned photomultiplier tubes (Hamamatsu H7422-40 GaAsP High Sensitivity PMTs and Hamamatsu H7422-50 GaAsP High Sensitivity red-extended PMT from Hamamatsu Photonics K.K.), a 25×, 1.05 NA, 2 mm working distance, water-immersion multiphoton objective (Olympus). For 4D analysis of cell migration, stacks of 7-15 square xy sections (512×512 pixel) sampled with 4 μm z spacing were acquired every 5-32 s for up to 2 hours, to provide image volumes that were 40 μm in depth and with an xy field of view variable between 100×100 μm² and 450×450 μm². Sequences of image stacks were transformed into volume-rendered, 4D time-lapse movies with Imaris (Bitplane). The 3D positions of the cell centroids were segmented by semi-automated cell tracking algorithm of Imaris. The semiautomatic surface-rendering module in Imaris (Bitplane) was used to create 3D volumetric surface objects corresponding either to individual cells or to the liver vascular system. Signal thresholds were determined using the Imaris Surface Creation module, which provides automatic threshold. The mean 3D velocity, the displacement (distance between the initial and the final position of a cell) and the confinement ratio (displacement over distance) were calculated from the x, y, and z coordinates of the cell centroids using custom designed scripts in Matlab (MathWorks).

Biochemical Analyses

The extent of hepatocellular injury was monitored by measuring sALT activity at multiple time points after treatment, as previously described (Guidotti, L. G. et al., 2015. Cell 161, 486-500).

Patients and Study Approval

A total of 34 patients with chronic HBV infection (HBsAg⁺) were included. The patients were subdivided into the disease categories Immune Tolerant (IT), Immune Active (IA) based on their clinical history. The 13 IT patients had no history of hepatitis (normal ALT) and are all positive for HBeAg. The 16 IA patients (5 HBeAg⁺, 12 HBeAg⁻) have or had previously signs of hepatic inflammation (ALT>40 IU/L), six of them are currently or were previously treated with nucleoside analogues. Blood donors were recruited from the viral hepatitis clinic at The Royal London Hospital. Written informed consent was obtained from all subjects. The study was conducted in accordance with the Declaration of Helsinki and approved by the Barts and the London NHS Trust local ethics review board and the NRES Committee London-Research Ethics Committee (reference 10/H0715/39) and by the Singapore National Healthcare Group ethical review board (DSRB 2008/00293).

Clinical and Virological Parameters

On recruitment to the study, viral serology and HBV DNA levels were tested. HBsAg, HBeAg and anti-HBe levels were measured with a chemiluminescent microparticle immunoassay (CMIA; Architect Assay, Abbott Diagnostics). HBV DNA levels in serum were quantified by real-time PCR (COBAS AmpliPrep/COBAS TaqMan HBV test v2.0; Roche Molecular Diagnostics) and HBV genotyping was performed by restriction fragment length polymorphism analysis of a pre-S amplicon, as described (Lindh, M., et al., 1998. J Virol Methods 72, 163-174).

HBV Peptide Library

Three libraries of 311-313 15-mer peptides overlapping by 10 amino acids were used to identify HBV-specific T cells. The peptides covered the entire sequence of HBV genotypes B, C and D (Gen Bank AF121243, AF 112063, AF 21241, respectively) and were purchased from Mimotopes. The purity of the peptides was above 80%, and their composition was confirmed by mass spectrometry analysis. Peptides were pooled as previously described (Tan, A. T. et al., 2008. J Virol 82, 10986-10997). The peptide libraries were matched to the HBV genotype of each patient. For patients infected with HBV genotype A or E, the peptide library of genotype D was used.

PBMC Isolation and T cell Culture

PBMCs were isolated from peripheral blood by Ficoll gradient and cryopreserved. Cells were thawed, and T cell lines were generated as follows: 20% of PBMCs were pulsed with 10 μg/ml of the overlapping HBV peptides for 1 hour at 37° C., subsequently washed, and cocultured with the remaining cells in AIM-V medium (Gibco; Thermo Fisher Scientific) supplemented with 2% AB human serum (Gibco; Thermo Fisher Scientific). T cell lines were cultured for 10 days, with or without the presence of 20 U/ml of recombinant IL-2 (R&D Systems).

ELISpot Assays

ELISpot assays for the detection of IFN-γ-producing cells were performed on in vitro expanded T cell lines using HBV peptides pooled into the following mixtures: X, core, envelope 1 (env 1), env 2, polymerase 1 (pol 1), pol 2, pol 3, pol 4. T cell lines were incubated overnight at 37° C. with pools of HBV peptides (1 μg/ml), where final DMSO concentrations did not exceed 0.2%. Medium was supplemented as before with or without 20 U/ml of recombinant IL-2. IFN-γ ELISpot assays (Millipore) were performed as previously described (Tan, A. T. et al., 2008. J Virol 82, 10986-10997).

Statistical Analyses

Results are expressed as mean±SEM. All statistical analyses were performed in Prism (GraphPad Software). Means between two groups were compared with two-tailed t test. Means among three or more groups were compared with one-way or two-way ANOVA with Bonferroni post-test. Patients data were analysed using the Wilcoxon paired t test.

### Example 2

Results

To shed light on the immune mechanisms underpinning the IL-2-mediated reinvigoration of intrahepatically-primed T cells, we initially took advantage of MUP-core transgenic mice that exclusively express a non-secretable version of the HBV core (but not envelope) protein in 100% of hepatocytes and have been used to model the hepatocellular Ag expression and intrahepatic priming events occurring upon neonatal HBV infection in humans (FIG. 30A). WT mice that are transduced with recombinant, replication-defective lymphocytic choriomeningitis (LCMV)-based vectors targeting both the HBV envelope protein and a non-secretable version of the HBV core protein (rLCMV-core/env) to intrahepatic professional Ag-presenting cells (i.e. Kupffer cells [KCs] and hepatic dendritic cells [DCs]) that are not naturally infected by HBV served as controls (FIG. 30A). Both groups of mice were injected with naïve CD8⁺ TCR transgenic T cells specific for epitopes contained within the envelope and core proteins of HBV (Env28 and Cor93 T_{N}, respectively) (FIG. 30A). Selected MUP-core mice received IL-2 coupled with non-neutralizing IL-2-specific monoclonal antibodies (S4B6) that enhance the half-life of IL-2 in vivo, one day after T_{N} injection (FIG. 30A). To test whether IL-2/anti-IL-2 immune complex (IL-2c) treatment had an exclusive direct effect on T_{N} or whether it required the presence of additional cells, we performed depletion experiments. We initially focused on KCs, as these cells were shown to be capable of inducing differentiation of CD8+ T cells into effector cells that are similar to those primed in secondary lymphoid organs. KCs were depleted through clodronate liposomes (CLL) injection, two days prior to T cell injection (FIG. 30A). This treatment effectively depletes KCs while sparing hepatic DCs (FIG. 30B-E). Consistent with previously published results, Cor93 and Env28 T_{N} transferred to WT mice injected with rLCMV-core/env differentiated into bona fide effector cells that formed tight clusters scattered throughout the liver lobules, Cor93 T cells transferred to MUP-core mice generated dysfunctional cells devoid of IFN-γ-producing ability that coalesced around portal tracts (FIG. 30F-H). IL-2c administration improved the capacity of Ag-specific Cor93 T cells to expand, differentiate into IFN-γ-producing cells and accumulate in clusters scattered throughout the liver lobules, but it had no effect on irrelevant Env28 T_{N} (FIG. 30F-H). Interestingly, optimal in vivo reinvigoration of intrahepatically primed Cor93 T cells required the presence of KCs, as shown by the effect of IL-2c treatment on T cell expansion, effector differentiation and intraparenchymal cluster formation in CLL-treated mice (FIG. 30F-H). Similar results were obtained when HBV replication-competent transgenic mice were used in place of MUP-core recipients and when WT IL-2 was used in place of IL-2c. Taken together, these results indicate that KCs, but not hepatic DCs, are required for optimal in vivo reinvigoration of intrahepatically-primed T cells by IL-2.

To confirm that hepatic DCs are not necessary for the optimal in vivo response to IL-2, we depleted this cell population by virtue of diphtheria toxin (DT) injection in MUP-core mice reconstituted with CD11c-DTR bone marrow (FIG. 30I). This treatment significantly decreased the number of hepatic DCs while sparing KCs (FIG. 30J-M). DC depletion did not affect the capacity of IL-2 to promote expansion, effector differentiation and intraparenchymal cluster accumulation of intrahepatically-primed Cor93 T cells (FIG. 30N-P). Similarly, other phagocytic cells such as neutrophils and monocytes were found not to be involved in the response to IL-2 as neutrophil depletion (via anti-Ly6G Abs) or combined neutrophil and monocyte depletion (via anti-Gr1 Abs) did not affect the in vivo reinvigoration of intrahepatically-primed T cells by IL-2 (FIG. 34).

We next investigated the effect of IL-2 treatment on KCs. Flow cytometric analyses revealed that KCs express all 3 subunits of the IL-2 receptor (CD25, CD122 and CD132) (FIG. 31A, B). To test whether the 3 subunits assemble into a functional IL-2 receptor capable of intracellular signal transduction, we isolated liver non parenchymal cells (LNPCs)—including KCs—from WT mice and stimulated them ex vivo with IL-2c (FIG. 31C). We observed a dose-dependent increase in pSTAT5 expression in KCs by flow cytometry (FIG. 31D) and this was confirmed by Western blot analyses (FIG. 31E). The data indicate that KCs express a functional IL-2 receptor capable of responding to IL-2 in vitro. To assess the consequences of IL-2 treatment on KCs in vivo, we treated WT mice with IL-2c for 48 hours and then performed RNA-seq analysis on FACS-sorted KCs (FIG. 31F, G). Using the limma R package, 4073 Differentially Expressed Genes (DEGs)—1515 up- and 2558 down-regulated—were identified as significantly regulated by IL-2c (FIG. 35). Functional enrichment of up-regulated genes showed an increased transcription of genes involved mainly in antigen presentation and proteasomal processing, ribosomal RNA processing and splicing, DNA replication and cell cycle, and mitochondrial oxidative metabolism (FIG. 31H, FIGS. 36 and 37). Down regulated genes were enriched in a number of different processes and pathways, but they did not cluster into well-defined biological groups and therefore we did not analyze them further (FIG. 36B). Amongst the clusters of up-regulated genes, we focused in particular on the antigen presentation pathway. The exogenous antigen presentation pathway includes ubiquitins, chaperones, MHC-I and proteasome subunits (FIG. 37A). Genes encoding for these protein families were overexpressed on KCs upon IL-2c treatment (FIG. 31I, K and FIG. 37B-F). In particular, the transcription factor Nirc5 and the Tap1 antigen transporter, both involved in the antigen presentation of exogenous peptides, along with the MHC-I and immunoproteasome subunits were found overexpressed on KCs upon IL-2c treatment (FIG. 31K). The MHC-I and co-stimulatory molecule up-regulation in KCs isolated from mice treated with IL-2c was confirmed at the protein level (FIG. 31L). Based on these results, we reasoned that in vivo treatment with IL-2c might increase the cross-presentation capacity of KCs. To test this possibility, we measured the capacity of in vitro differentiated Cor93-specific effector CD8⁺ T cells to produce IFN-γ (as an indirect measure of Ag recognition) upon incubation with KCs isolated from HBV replication-competent transgenic mice that were treated or not with IL-2c (FIG. 31M). Baseline KC cross-presentation of hepatocellular Ags in this experimental system at steady state is negligible; however, in vivo treatment with IL-2c slightly but significantly increased the cross-presentation capacity of KCs (FIG. 31N, O). We then assessed the ability of KCs isolated from IL-2-treated WT mice to cross-prime HBV-specific naïve CD8⁺ T cells upon exposure to HBV (FIG. 31P). When compared to KCs isolated from PBS-treated mice, KCs exposed in vivo to IL-2 induced a higher proliferation of HBV-specific naïve CD8⁺ T cells upon in vitro culture (FIG. 31Q, R). Finally, to assess the in vivo relevance of our findings, we generated MUP-core mice whose KCs lack Transporter associated with Antigen Processing 1 (TAP-1) and therefore cannot express MHC-I and present Ags to CD8⁺ T cells (FIG. 31S). This was achieved by injection of either WT or TAP-1^{-/-} bone marrow into irradiated MUP-core mice, followed by CLL treatment to deplete the residual radio-resistant Kupffer cells and allow the complete reconstitution of the entire KC compartment with bone marrow-derived cells (FIG. 40). HBV-specific T_{N} injected into MUP-core mice whose hematopoietic cells (including KCs) lacked MHC-I had a much lower response to IL-2c than did T_{N} injected into mice carrying Ag presentation-competent KCs (FIG. 31T, U). Taken together, these results indicate that optimal reinvigoration of intrahepatically primed CD8⁺ T cells by IL-2 requires the capacity of KCs to cross-present hepatocellular Ags.

Next, we asked whether IL-2 acts homogenously on all KCs or whether a specific KC subset is responsible for the observed effect. To this end, we performed single-cell RNA-sequencing (scRNA-seq) on Kupffer cells isolated from WT mice. Unbiased clustering revealed two distinct populations of KCs (referred to as KC1 and KC2, respectively) that can be distinguished using a number of markers such as CD206 and ESAM (FIG. 32A-C). KC2 were found to be CD206^{high} ESAM^{high} and represent ˜15-30% of total KCs (FIG. 32A, B).

Imaging analyses confirmed the presence of two distinct KC subsets but failed to reveal a preferential zonal distribution for either subset (FIG. 32C). RNA-seq analyses on KC1 and KC2 sorted from WT mice revealed that KC2 are enriched in IL-2 signaling components (IL-2 receptor subunits and molecules implicated in intracellular signal transduction) (FIG. 32D, E, FIG. 41). Higher expression of the IL-2 receptor subunits, MHC-I and co-stimulatory molecules in KC2 was confirmed at the protein level by FACS analysis (FIG. 32F-J). Together, the data suggest that KC2 are better equipped to respond to IL-2 and increase their capacity to cross-present hepatocellular Ags. If this were the case, one might predict that IL-2 treatment might render KC2 more sensitive than KC1 to CD8⁺ T cell-mediated elimination. To test this hypothesis, we treated HBV replication competent transgenic mice with IL-2c 24 hours after Cor93 injection and checked the KC1/KC2 ratio 4 days later. Consistent with the hypothesis that IL-2 preferentially increases the capacity of KC2 to cross-present hepatocellular Ags and thus renders them more sensitive to CD8⁺ T cell-mediated killing, we found that KC2 almost completely disappeared in Cor93 T cell-injected HBV transgenic mice treated with IL-2c (FIG. 32K, L). Importantly, neither IL-2c treatment alone (in the absence of HBV-specific T cell transfer) nor acute liver inflammation (induced by HBV-specific effector CD8⁺ T cell injection in HBV transgenic mice) altered the KC1/KC2 ratio (FIG. 42).

We next sought to generate a model where KC2 could be selectively depleted. We took advantage of the observation that KC2 (but not KC1) express the endothelial cell marker VE-cadherin (encoded by Cdh5). We therefore injected Cdh5^{cre/ERT2}; R26^{IDTR} bone marrow into irradiated MUP-core mice, depleted the residual radio-resistant KCs by CLL to allow the complete reconstitution of the entire KC compartment with bone marrow-derived cells, induce DTR expression in KC2 by tamoxifen administration and, finally, depleted KC2 by DT injection prior to Cor93 T_{N} transfer followed by IL-2c treatment (FIG. 33A, B). DT treatment caused a ˜4-fold decrease in KC2 and resulted in a lower ability of HBV-specific T cells to proliferate and differentiate into cytotoxic effector cells clustered throughout the liver lobule in response to IL-2 (FIG. 33C-F). The data indicate that KC2 are required for the optimal reinvigoration of intrahepatically primed T cells by IL-2.

We have delineated the cellular and molecular mechanisms by which IL-2 enhances proliferation and antiviral activity of intrahepatically primed T cells. Whereas the baseline cross-presentation of hepatocellular Ags by KCs is a rather inefficient process, it can be increased by the therapeutic administration of IL-2. Our results identify a subset of KC—referred to as KC2—that preferentially respond to IL-2 to cross-present hepatocellular Ags to CD8⁺ T cells. Our data do not rule out a direct effect of IL-2 on T cells; however, they show that optimal in vivo reinvigoration of intrahepatically primed T cells by IL-2 depends on the presence of KC2.

Materials and Methods

Mice C57BL/6, CD45.1 (inbred C57BL/6), Balb/c, Thy1.1 (CBy.PL(B6)-Thy^{a}/ScrJ), β-actin-GFP [C57BL/6-Tg(CAG-EGFP)1Osb/J], β-actin-DsRed [B6.Cg-Tg(CAG-DsRed*MST)1Nagy/J], Ai6(RCL-ZsGreen) [B6.Cg-Gt(ROSA)26Sortm6(CAG-ZsGreen1)Hze/J], Ai14(RCL-tdT)-D [B6.Cg-Gt(ROSA)26Sortm14(CAG-tdTomato)Hze/J], Tap1-deficient (B6.12952-Tap1^{tm1Arp}/J), TCR-I [B6.Cg-Tg(TcraY1, TcrbY1)416Tev/J], CD11c-DTR [B6.FVB-1700016L2Rik^{Tg(tgax-DTR/EGFP)57Lan}/J]. CD122^{flox} [B6.129S1-Il2rbtm1Ukl/J], ROSA26iDTR [C57BL/6-Gt(ROSA)26Sortm1(H BEG F)Awai/J], CD4-cre [B6.Cg-Tg(Cd4-cre)1Cwi/BfluJ], Cdh5-creERT2 [Tg(Cdh5-cre/ERT2)1 Rha] mice were purchased from Charles River or The Jackson Laboratory. CD25^{flox} [B6(12954)-Il2ratm1c(EUCOMM)Wtsi/TrmaJ] mice were provided by G. Gasteiger. MUP-core transgenic mice (lineage MUP-core 50 [MC50], inbred C57BL/6, H-2^{b}) express the HBV core protein in 100% of the hepatocytes under the transcriptional control of the mouse major urinary protein (MUP) promoter, have been previously described. HBV replication-competent transgenic mice (lineage 1.3.32, inbred C57BL/6, H-2^{b}), that express all of the HBV antigens and replicate HBV in the liver at high levels without any evidence of cytopathology, have been previously described. In indicated experiments, MUP-core and HBV replication-competent transgenic mice were used as C57BL/6×Balb/c H-2^{bxd} F1 hybrids. Cor93 TCR transgenic mice (lineage BC10.3, inbred CD45.1), in which >98% of the splenic CD8⁺ T cells recognize a K^{b}-restricted epitope located between residues 93-100 in the HBV core protein (MGLKFRQL), have been previously described. Env28 TCR transgenic mice (lineage 6C2.36, inbred Thy1.1 Balb/c), in which ˜83% of the splenic CD8⁺ T cells recognize a L^{d}-restricted epitope located between residues 28-39 of HBsAg (IPQSLDSWWTSL), have been previously described. For imaging experiments Cor93 transgenic mice were bred against both β-actin-GFP, while Env28 transgenic mice were bred against β-actin-DsRed mice that were previously back-crossed more than 10 generations against Balb/c. Bone marrow (BM) chimeras were generated by irradiation of MUP-core or C57BL/6 mice with one dose of 900 rad and reconstitution with the indicated BM; mice were allowed to reconstitute for at least 8 weeks before use. In some experiments, to achieve full reconstitution of Kupffer cells from donor-derived BM, mice were injected with 200 μl of clodronate-containing liposomes 28 and 31 days after BM injection. In some experiments, to induce the expression of the -cre, mice were treated with 5 mg of Tamoxifen by gavage in 200 μl of corn oil one week before further manipulations. Mice were housed under specific pathogen-free conditions and used at 8-10 weeks of age. In all experiments, mice were matched for age, sex and (for the 1.3.32 animals) serum HBeAg levels before experimental manipulations. All experimental animal procedures were approved by the Institutional Animal Committee of the San Raffaele Scientific Institute and are compliant with all relevant ethical regulations.

Naïve T Cell Isolation, Adoptive Transfer and In Vivo Treatments

CD8⁺ T cells from the spleens of Cor93 and Env28 transgenic mice were purified by negative immunomagnetic sorting (Miltenyi Biotec). Mice were adoptively transferred with 5×10⁶, or 1×10⁶ CD8⁺ T cells. In selected experiments, WT or MUP-core mice were lethally irradiated and reconstituted with BM from CD11c-DTR mice; dendritic cells were subsequently depleted by injecting i.p. 20 ng/g of diphtheria toxin (Millipore) every other day starting from 3 days before T cell transfer. In indicated experiments, Kupffer cells were depleted by intravenous injection of clodronate-containing liposomes 2 days prior to T cell injection, as described or by injecting i.p. 20 ng/g of diphtheria toxin (Sigma) 1 day prior to T cell transfer. In selected experiments, KC2 were depleted by injecting i.p. 20 ng/g of diphtheria toxin (Sigma) 3 days and 1 day prior to T cell transfer. IL-2/anti-IL-2 complexes (IL-2c) were prepared by mixing 1.5 μg of rIL-2 (R&D Systems) with 50 μg anti-IL-2 mAb (clone S4B6-1, BioXcell) per mouse, as previously described. Mice were injected with IL-2c i.p. one day after T cell transfer, unless otherwise indicated.

Cell Isolation and Flow Cytometry

Single-cell suspensions of livers, spleens and blood were generated. Kupffer cell isolation was performed using standard protocols. Cell viability was assessed by staining with Viobility™ 405/520 fixable dye (Miltenyi) or DAPI. Abs used included: anti-CD3 (clone: 145-2C11, Cat #562286, BD Biosciences), anti-CD11b (clone: M1/70, Cat #101239), anti-CD19 (clone: 1 D3, Cat #562291 BD Biosciences), anti-CD25 (clone: PC61, Cat #102015), anti-CD31 (clone: 390, Cat #102427), anti-CD45 (clone: 30-F11, Cat #564279 BD Biosciences), anti-CD64 (clone: X54-5/7.1, Cat #139311), anti-F4/80 (clone: BM8, Cat #123117), anti-I-A/I-E (clone: M5/114.15.2, Cat #107622), anti-TIM4 (clone: RTM4-54 Cat #130010), anti-CD69 (clone: H1.2F3, Cat #104517), anti-CD45.1 (clone: A20, Cat #110716), anti-IFN-γ (clone: XMG1.2, Cat #557735 BD Biosciences), anti-CD11c (clone: N418, Cat #117308), anti-1-Ab (clone: AF6-120.1, Cat #116420), anti-PD-1 (clone: J43, Cat #17-9985 eBioscience), anti-NK1.1 (clone: PK136, Cat #108706), anti-NKp46 (clone: 29A1.4, Cat #137623), anti-Stat5 pY694 (clone: 47, Cat #612599 BD Biosciences), CD122 (clone IM-B1 Cat #123210), CD132 (clone TUgm2 Cat #132306), CD40 (clone 3/23 Cat #558695 BD Biosciences), CD80 (clone 1610A1 Cat #553769 BD Biosciences), H2-Kb (clone AF6-88.5 Cat #742861, BD Biosciences). All Abs were purchased from BioLegend, unless otherwise indicated. Recombinant dimeric H-2L^{d}:Ig and H-2K^{b}:Ig fusion proteins (BD Biosciences) complexed with peptides derived from HBsAg (Env28-39) or from HBcAg (Cor93-100), respectively, were prepared according to the manufacturer's instructions. Flow cytometry staining for phosphorylated STAT5 was performed using Phosflow™ Perm Buffer III (Cat #558050, BD Bioscience), following the manufacturer's instructions.

All flow cytometry analyses were performed in FACS buffer containing PBS with 2 mM EDTA and 2% FBS on a FACS CANTO or LSRII (BD Biosciences) and analysed with FlowJo software (Treestar).

Cell Purification

Single-cell suspensions from spleens and livers were stained with Viobility 405/520 fixable dye (Miltenyi) or DAPI, with PB-conjugated anti-CD8a (clone 53-6.7) and PE-conjugated anti-CD45.1 Abs. Live, lineage negative (CD3, CD19, Ly6-G, CD49b), CD45⁺, CD31⁻, CD11b^{int}, F4/80⁺, MHCII⁺, TIM4⁺ and CD206⁻, ESAM⁻ (KC₁) or CD206⁺, ESAM⁺ (KC₂) cells were sorted with a 100 micron nozzle at 4° C. on a FACSAria Fusion (BD) cell sorter in a buffer containing PBS with 2% FBS. Cells were always at least 98% pure. In indicated experiments, F4/80⁺ cells were purified from liver NPCs by negative immunomagnetic sorting (Miltenyi Biotec, #130-110-443).

RNA Purification and RNA-seq Library Preparation

Total KCs, KC₁ and KC₂ were FACS-sorted from liver NPCs. Cells were lysed in ReliaPrep™ RNA Cell Miniprep System (Promega #Z6011) and total RNA was isolated following manual extraction. DNA digestion was performed with TURBO DNA-Free™ Kit (Invitrogen #AM1907). RNA was quantified with Qubit™ RNA HS Assay Kit (Invitrogen # Q32852) and analysis of its integrity was assessed with Agilent RNA 6000 Pico Kit (Agilent #5067-1513) on a Bioanalyser instrument.

6 RNA samples of sorted KC1 and KC2, were processed with the “SMART-seq Ultra Low Input 48” library protocol in order to obtain 30.0M clusters of fragments of 1×100 nt of length through NovaSeq 6000 SP Reagent Kit (100 cycles).

Raw reads were aligned to mouse genome build GRCm38 using STAR aligner. Read counts per gene were then calculated using featureCounts (part of the R subread package) based on GENCODE gene annotation version M16.

Read counts were Log 2 transformed transcripts per million (log 2 TPM) normalisation were produced to account for transcript length and the total number of reads. Only genes with a TPM value higher than 1 in 3 samples or more were considered for following analysis.

Differentially Expressed Genes (DEGs) between KC2 and KC1 samples, were identified by generating a linear model using LIMMA R package. Only DEGs with an adjusted P value <0.05 (using Benjamini Hochberg method) and a |log FC|>1 were selected for further analysis.

RNA-Seq Transcriptome Analysis

Raw reads were aligned to mouse genome build GRCm38 using STAR aligner. Read counts per gene were then calculated using featureCounts (part of the R subread package) based on GENCODE gene annotation version M22. Log 2 transformed transcripts per million (log 2 TPM) normalisation were produced to account for transcript length and the total number of reads. Only genes with a TPM value higher than 1 in 4 samples or more were considered for following analysis. Differentially Expressed Genes (DEGs) between samples treated with IL2c and PBS, were identified by generating a linear model using LIMMA R package. Only DEGs with an adjusted P value <0.05 (using Benjamini Hochberg method) were selected for further analysis.

Functional Enrichment Analysis

Of the 4073 significant (FDR<0.05) identified DEGs between Control (+PBS) and treated (+IL-2c) samples, 1515 were up-regulated and 2558 were down-regulated. Those were subject to a functional enrichment analysis using the EnrichR R package. Both the up- and the down-regulated DEGs were checked for any biological signature enrichment in both the Gene Ontology Biological Process Database (2018) and the Kyoto Encyclopedia of Genes and Genomes for Mouse (2019). After merging the results for the two databases, 858 significant (FDR<0.05) Terms were identified, of which 428 from the up-regulated DEGs and 430 from the down-regulated ones.

In order to select the top enriched Terms, only ones with an high Combined Score (−log(p-value)*Odds Ratio) were considered. Based on the distribution of the Combined Score in the up-regulated Terms and in the down-regulated ones, a threshold of 100 was chosen for the former, while a threshold of 30 for the latter.

Clustering of Up-Regulated Terms

For visualization and analysis, both up-regulated and down-regulated Terms were subject to a clustering algorithm, in order to identify the most prominent biological signatures. Briefly, a Jaccard Index Similarity score was calculated for each pair set of Terms, based on the DEGs annotated for each Term, using an in-house developed script. Next, Terms were clustered using a hierarchical clustering method, using as distance measure the Pearson correlation between the calculated Jaccard Index Similarity scores. An arbitrary number of clusters were selected and manually annotated based on the Terms present. To visualize the result, the pheatmap R package was used.

Radar Plots Visualization

Radar plots where generated using the fmsb R package. Different set of genes where selected based on literature analysis, defining different biological processes. For each category, the mean of the TPM expression for each gene within samples (separately for Control and Treated samples) was calculated. Next, the mean between all the genes belonging to a category was calculated, and used as the value to represent the dimension in the radar plot.

Network Plot Visualization

Network plot was built using Cystoscape software (V 3.8.0 for MacOS). Briefly, starting from EnrichR tables, a matrix defining every pair of Term—Gene was generated, and used as a Node list input for Cytoscape.

Purification of Viral Nucleic Acids from Serum

20 μl of serum were incubated for 2 hours at 37° C. with 180 μl IsoHi Buffer (150 mM NaCl, 0.5% NP40, 10 mM Tris pH 7.4), 5 mM CaCl₂), 5 mM MgCl2, 1U DNasel (Life Technologies), 5U Micrococcal Nuclease (Life Technologies). The digestion was stopped by the addition of 20 mM EDTA pH 8.0 and viral nucleic acid purification performed with the QIAmp MiniElute Virus Spin Kit (Qiagen, Cat #57704), according to the manufacturer's instructions.

RT-qPCR

Total RNA was extracted from frozen livers using ReliaPrep™ RNA Tissue Miniprep System (Promega), according to the manufacturer's instructions, genomic DNA contamination was removed using Ambion® TURBO DNA-Free™ DNase. 1 μg of total RNA was reverse transcribed with Superscript IV Vilo (Life Technologies) prior to qPCR analysis for mouse 112 (TaqMan Mm00434256, Life Technologies), ifng (TaqMan Mm01168134, Life Technologies), HBV core (forward TACCGCCTCAGCTCTGTATC, reverse CTTCCAAATTAACACCCACCC, probe TCACCTCACCATACTGCACTCAGGCAA). Reactions were run and analysed on Quant Studio 5 instrument (Life Technologies). For viremia quantification, a standard curve was drawn using plasmid DNA. All experiments were performed in triplicate and normalized to the reference gene GAPDH.

Western Blot Analysis

Primary Abs include anti-STAT5 and anti-pSTAT5 (Tyr694) (rabbit; Cell Signalling #8215). Secondary Ab include horseradish peroxidase-conjugated goat anti-rabbit IgG (Jackson ImmunoResearch). Reactive proteins were visualized using a Clarity Western ECL substrate kit (Bio-Rad), and exposure was performed using UVltec (Cambridge MINI HD, Eppendorf). Images were acquired by NineAlliance software. Band quantification was performed with ImageJ software on 16-bit images and normalized on the matching housekeeping protein as a loading control. Each lane corresponds to a different mouse.

Southern Blot Analysis

Southern blot analysis on total DNA isolated from frozen livers (left lobe) was performed as previously described.

Confocal Immunofluorescence Histology and Histochemistry

For confocal images, KC1 and KC2 were labelled by i.v. injection of 2 ug F4/80 Alexa fluor 488 (Biolegend #123120) and 2 ug CD206-APC (Biolegend 141708) into WT C57616 mice 10 min prior to sacrificing the animal. The liver was fixed overnight in PBS with 4% paraformaldehyde and subsequently incubated for 24 h in PBS with 30% sucrose. Next, liver lobes were embedded in O.C.T (Killik Bio-Optica 05-9801), and cut at −14° C. into 60 um thick sections with a cryostat. Sections were blocked for 15 min with blocking buffer (PBS, 0.5% BSA, 0.3% Triton) and stained for 1 h at RT with CD38 Alexa fluor 594 (Biolegend 102725) in wash/stain buffer (PBS, 0.2% BSA, 0.1% triton). Then, sections were washed twice for 5 min, stained with DAPI (Sigma 28718-90-3) for 5 min, washed again and mounted for imaging with Fluorosave™ Reagent (Millipore 345789-20ML). Image acquisition was performed with an 63×oil-immersion or 20× objective at an SP5 confocal microscope (Leica Microsystem). The following primary Abs were used for staining: anti-F4/80 (BM8, Invitrogen), anti-cytokeratin 7 (EPR17078, Abcam), anti-Lyve-1 (NB600-1008, Novus Biological), anti-HBcAg (polyclonal, Dako). The following secondary Abs were used for staining: Alexa Fluor 488-, Alexa Fluor 514-, Alexa Fluor 568-, or Alexa Fluor 647-conjugated anti-rabbit or anti-rat IgG (Life Technologies). Images were acquired on inverted Leica microscopes (TCS STED CW SP8, Leica Microsystems,) with a motorized stage for tiled imaging. To minimize fluorophore spectral spillover, we used the Leica sequential laser excitation and detection modality.

For H&E and HBcAg immunohistochemistry, livers were perfused with PBS, harvested in Zn-formalin and transferred into 70% ethanol 24 hours later. Tissue was then processed, embedded in paraffin and stained as previously described. Bright-field images were acquired through an Aperio Scanscope System CS2 microscope and an ImageScope program (Leica Biosystem) following the manufacturer's instructions.

Biochemical Analyses

The extent of hepatocellular injury was monitored by measuring sALT activity at multiple time points after treatment.

All publications mentioned in the above specification are herein incorporated by reference. Various modifications and variations of the disclosed vectors, compositions, products, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. An interleukin which binds to IL-2 receptor (IL-2R), or a nucleotide sequence encoding therefor, wherein the interleukin or nucleotide sequence is adapted to be targeted to the liver.

2. The interleukin of claim 1, wherein the interleukin is comprised in a nanoparticle or liposome.

3. The interleukin of claim 2, wherein the nanoparticle or liposome comprises a liver-specific ligand.

4. The interleukin of claim 1, wherein the nucleotide sequence encoding the interleukin is in the form of a vector adapted for liver-specific expression of the nucleotide sequence.

5. A vector comprising a nucleotide sequence encoding an interleukin which binds to IL-2 receptor (IL-2R), wherein the vector is adapted for liver-specific expression of the nucleotide sequence.

6. The vector or interleukin of claim 4 or 5, wherein the nucleotide sequence is operably linked to one or more expression control sequences for liver-specific expression.

7. The vector or interleukin of any one of claims 4-6, wherein the nucleotide sequence is operably linked to one or more miR-142, miR-155 and/or miR-223 target sequences, preferably wherein the nucleotide sequence is operably linked to one or more miR-142 target sequences.

8. The vector or interleukin of any one of claims 4-7, wherein the vector comprises 2, 3 or 4 miR-142, miR-155 and/or miR-223 target sequences operably linked to the nucleotide sequence.

9. The vector or interleukin of any one of claims 4-8, wherein the vector comprises a liver-specific promoter and/or enhancer operably linked to the nucleotide sequence, optionally wherein the vector comprises a hepatocyte-specific promoter and/or enhancer operably linked to the nucleotide sequence.

10. The vector or interleukin of claim 9, wherein the hepatocyte-specific promoter is selected from the group consisting of an ET promoter, albumin promoter, transthyretin promoter, alpha1-antitrypsin promoter and apoE/alpha1-antitrypsin promoter, preferably wherein the promoter is an ET promoter.

11. The vector or interleukin of any preceding claim, wherein the interleukin is selected from the group consisting of IL-2, IL-7 and IL-15, preferably wherein the interleukin is IL-2.

12. The vector or interleukin of any one of claims 4-11, wherein the vector comprises: (a) a nucleotide sequence encoding IL-2; (b) a nucleotide sequence encoding IL-7; and/or (c) a nucleotide sequence encoding IL-15, preferably wherein each of (a)-(c) is operably linked to one or more expression control sequences for liver-specific expression.

13. The vector or interleukin of any one of claims 4-12, wherein the vector is a viral vector and/or an RNA vector.

14. The vector or interleukin of any one of claims 4-13, wherein the vector is a retroviral, lentiviral, adenoviral or adeno-associated viral (AAV) vector, preferably a lentiviral vector.

15. The vector or interleukin of any one of claims 4-14, wherein the vector is an integration-defective lentiviral vector (IDLV).

16. The vector or interleukin of any one of claims 4-15, wherein the vector is in the form of a viral vector particle.

17. The vector or interleukin of any one of claims 4-13, wherein the vector is in the form of a liposome or lipid nanoparticle, preferably wherein the vector is an RNA vector.

18. A composition or kit comprising two or more interleukins selected from the group consisting of:
(a) the interleukin of any one of claims 1-3, wherein the interleukin is IL-2;
(b) the interleukin of any one of claims 1-3, wherein the interleukin is IL-7; and
(c) the interleukin of any one of claims 1-3, wherein the interleukin is IL-15,
wherein at least two interleukins are selected from different groups (a), (b) or (c).

19. A composition or kit comprising two or more vectors selected from the group consisting of:
(a) the vector or interleukin of any one of claims 4-17, wherein the vector comprises a nucleotide sequence encoding IL-2;
(b) the vector or interleukin of any one of claims 4-17, wherein the vector comprises a nucleotide sequence encoding IL-7; and
(c) the vector or interleukin of any one of claims 4-17, wherein the vector comprises a nucleotide sequence encoding IL-15,
wherein at least two vectors are selected from different groups (a), (b) or (c).

20. A pharmaceutical composition comprising the interleukin, vector or composition of any preceding claim, and a pharmaceutically-acceptable carrier, diluent or excipient.

21. The pharmaceutical composition of claim 20, wherein the pharmaceutical composition further comprises a population of T cells, preferably wherein the T cells express a chimeric antigen receptor (CAR) or a T cell receptor (TCR), which binds to a hepatitis virus antigen.

22. A interleukin, vector, composition or kit according to any one of claims 1-21 for use in treating or preventing a viral liver infection and/or hepatocellular carcinoma.

23. The interleukin, vector, composition or kit for use according to claim 22, wherein the viral liver infection is a hepatitis virus infection, preferably a chronic hepatitis virus infection.

24. The interleukin, vector, composition or kit for use according to claim 22 or 23, wherein the viral liver infection is an hepatitis B virus (HBV) and/or hepatitis C virus (HCV) infection, preferably an HBV infection.

25. The interleukin, vector, composition or kit for use according to any one of claims 22-24, wherein the interleukin, vector or composition is locally administered to a subject, preferably to a subject's liver.

26. The interleukin, vector, composition or kit for use according to any one of claims 22-25, wherein the interleukin, vector or composition is administered as part of an adoptive T cell therapy.

27. The interleukin, vector, composition or kit for use according to any one of claims 22-26, wherein the interleukin or vector is administered simultaneously, separately or sequentially with a population of T cells, preferably wherein the T cells express a chimeric antigen receptor (CAR) or a T cell receptor (TCR), which binds to a hepatitis virus antigen.

28. A product comprising (a) the interleukin, vector or composition of any one of claims 1-21; and (b) a population of T cells, as a combined preparation for simultaneous, separate or sequential use in therapy, preferably wherein the T cells express a chimeric antigen receptor (CAR) or a T cell receptor (TCR), which binds to a hepatitis virus antigen.

29. The product for use according to claim 28, wherein the product is for use in treating or preventing a viral liver infection or hepatocellular carcinoma.

30. The product for use according to claim 29, wherein the viral liver infection is a hepatitis virus infection, preferably a chronic hepatitis virus infection.

31. The product for use according to claim 29 or 30, wherein the viral liver infection is an hepatitis B virus (HBV) and/or hepatitis C virus (HCV) infection, preferably an HBV infection.

32. The product for use according to any one of claims 28-31, wherein the vector and/or population of T cells is locally administered to a subject, preferably to a subject's liver.
